SEQUENCE LISTING (51 sequences)

SEQ ID NO: 1 - DNA, 2058 bp, Homo sapiens
  source 1..2058: mol_type = unassigned DNA
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 2 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - DNA, 1923 bp, Homo sapiens
  source 1..1923: mol_type = unassigned DNA
gcccctttctttctcctcgtcggcccgagagcaggaacacgataacgaaggaggcccaacttcattcaataaggagcctgacggatttatcccagacggtagaacaaaaggaagaatattgatggattttaaaccagagtttttaaagagcttgagaatacggggaaattaatttgttctcctacacacatagatagggtaaggttgtttctgatgcagctgagaaaaatgcagaccgtcaaaaaggagcaggcgtctcttgatgccagtagcaatgtggacaagatgatggtccttaattctgctttaacggaagtgtcagaagactccacaacaggtgaggacgtgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagtttcaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgtcatttgcaatagagcagtccattttgtattatgctgaattttcactggacctgtgatgtcatttcactgtgatgtgcacatgttgtctgtttggtgtctttttgtgcacagattatgatgaagattagattgtgttatcactctgcctgtgtatagtcagatagtcatatgcgtaaggctgtatatattaagnttttatttttgttgttctattataaagtgtgtaagttaccagtttcaataaaggattggtgacaaacacagaaaaaaaaaaaaaaaaaaa

SEQ ID NO: 4 - protein, 462 aa, Homo sapiens [sequence omitted]

SEQ ID NO: 5 - DNA, 2793 bp, Homo sapiens
  source 1..2793: mol_type = unassigned DNA
gggcacgaggcgctccctgggatcacatggtacctgctccagtgccgcgtgcggcccgggaaccctgggctgctggcgcctgcgcagagccctctgtcccagggaaaggctcgggcaaaaggcggctgagattggcagagtgaaatattactgccgagggaacgtagcagggcacacgtctcgcctctttgcgactcggtgccccgtttctccccatcacctacttacttcctggttgcaacctctcttcctctgggacttttgcaccgggagctccagattcgccaccccgcagcgctgcggagccggcaggcagaggcaccccgtacactgcagagacccgaccctccttgctaccttctagccagaactactgcaggctgattccccctacacactctctctgctcttcccatgcaaagcagaactccgttgcctcaacgtccaacccttctgcagggctgcagtccggccaccccaagaccttgctgcagggtgcttcggatcctgatcgtgagtcgcggggtccactccccgcccttagccagtgcccagggggcaacagcggcgatcgcaacctctagtttgagtcaaggtccagtttgaatgaccgctctcagctggtgaagacatgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctggccccctgagccttcttgtaaatacctgcctccctcccccatcaccgaacttcccctcctcccctatttaaaccactctgtctcccccacaaccctcccctggccctctgatttgttctgttcctgtctcaaatccaatagttcacagctgagctggcttcaaaaaaaaaaaaaaaaaa

SEQ ID NO: 6 - protein, 614 aa, Homo sapiens
MTTLDSNNNTGGVITYIGSSGSSPSRTSPESLYSDNSNGSFQSLTQGCPTYFPPSPTGSLTQDPARSFGSIPPSLSDDGSPSSSSSSSSSSSSFYNGSPPGSLQVAMEDSSRVSPSKSTSNITKLNGMVLLCKVCGDVASGFHYGVHACEGCKGFFRRSIQQNIQYKRCLKNENCSIVRINRNRCQQCRFKKCLSVGMSRDAVRFGRIPKREKQRMLAEMQSAMNLANNQLSSQCPLETSPTQHPTPGPMGPSPPPAPVPSPLVGFSQFPQQLTPPRSPSPEPTVEDVISQVARAHREIFTYAHDKLGSSPGNFNANHASGSPPATTPHRWENQGCPPAPNDNNTLAAQRHNEALNGLRQAPSSYPPTWPPGPAHHSCHQSNSNGHRLCPTHVYAAPEGKAPANSPRQGNSKNVLLACPMNMYPHGRSGRTVQEIWEDFSMSFTPAVREVVEFAKHIPGFRDLSQHDQVTLLKAGTFEVLMVRFASLFNVKDQTVMFLSRTTYSLQELGAMGMGDLLSAMFDFSEKLNSLALTEEELGLFTAVVLVSADRSGMENSASVEQLQETLLRALRALVLKNRPLETSRFTKLLLKLPDLRTLNNMHSEKLLSFRVDAQ

SEQ ID NO: 7 - DNA, 1740 bp, Homo sapiens
  source 1..1740: mol_type = unassigned DNA
atggaggtgaatgcaggaggtgtgattgcctatatcagttcttccagctcagcctcaagccctgcctcttgtcacagtgagggttctgagaatagtttccagtcctcctcctcttctgttccatcttctccaaatagctctaattctgataccaatggtaatcccaagaatggtgatctcgccaatattgaaggcatcttgaagaatgatcgaatagattgttctatgaaaacaagcaaatcgagtgcacctgggatgacaaaaaatcatagtggtgtgacaaaatttagtggcatggttctactgtgtaaagtctgtggggatgtggcgtcaggattccactatggagttcatgcttgcgaaggctgtaagggtttctttcggagaagtattcaacaaaacatccagtacaagaagtgcctgaagaatgaaaactgttctataatgagaatgaataggaacagatgtcagcaatgtcgcttcaaaaagtgtctgtctgttggaatgtcaagagatgctgttcggtttggtcgtattcctaagcgtgaaaaacagaggatgctaattgaaatgcaaagtgcaatgaagaccatgatgaacagccagttcagtggtcacttgcaaaatgacacattagtagaacatcatgaacagacagccttgccagcccaggaacagctgcgacccaagccccaactggagcaagaaaacatcaaaagctcttctcctccatcttctgattttgcaaaggaagaagtgattggcatggtgaccagagctcacaaggatacctttatgtataatcaagagcagcaagaaaactcagctgagagcatgcagccccagagaggagaacggattcccaagaacatggagcaatataatttaaatcatgatcattgcggcaatgggcttagcagccattttccctgtagtgagagccagcagcatctcaatggacagttcaaagggaggaatataatgcattacccanatggccatgccatttgtattgcaaatggacattgtatgaacttctccaatgcttatactcaaagagtatgtgatagagttccgatagatggattttctcagaatgagaacaagaatagttacctgtgcaacactggaggaagaatgcatctggtttgtccaatgagtaagtctccatatgtggatcctcataaatcaggacatgaaatctgggaagaattttcgatgagcttcactccagcagtgaaagaagtggtggaatttgcaaagcgtattcctgggttcagagatctctctcagcatgaccaggtcaaccttttaaaggctgggacttttgaggttttaatggtacggttcgcatcattatttgatgcaaaggaacgtactgtcacctttttaagtggaaagaaatatagtgtggatgatttacactcaatgggagcaggggatctgctaaactctatgtttgaatttagtgagaagctaaatgccctccaacttagtgatgaagagatgagtttgtttacagctgttgtcctggtatctgcagatcgatctggaatagaaaacgtcaactctgtggaggctttgcaggaaactctcattcgtgcactaaggaccttaataatgaaaaaccatccaaatgaggcctctatttttacaaaactgcttctaaagttgccagatcttcgatctttaaacaacatgcactctgaggagctcttggcctttaaagttcacccttaa

SEQ ID NO: 8 - protein, 578 aa, Homo sapiens
MEVNAGGVIAYISSSSSASSPASCHSEGSENSFQSSSSSVPSSPNSSNSDTNGNPKNGDLANIEGILKNDRIDCSMKTSKSSAPGMTKNHSGVTKFSGMVLLCKVCGDVASGFHYGVHACEGCKGFFRRSIQQNIQYKKCLKNENCSIMRMNRNRCQQCRFKKCLSVGMSRDAVRFGRIPKREKQRMLIEMQSAMKTMMNSQFSGHLQNDTLVEHHEQTALPAQEQLRPKPQLEQENIKSSSPPSSDFAKEEVIGMVTRAHKDTFMYNQEQQENSAESMQPQRGERIPKNMEQYNLNHDHCGNGLSSHFPCSESQQHLNGQFKGRNIMHYPXGHAICIANGHCMNFSNAYTQRVCDRVPIDGFSQNENKNSYLCNTGGRMHLVCPMSKSPYVDPHKSGHEIWEEFSMSFTPAVKEVVEFAKRIPGFRDLSQHDQVNLLKAGTFEVLMVRFASLFDAKERTVTFLSGKKYSVDDLHSMGAGDLLNSMFEFSEKLNALQLSDEEMSLFTAVVLVSADRSGIENVNSVEALQETLIRALRTLIMKNHPNEASIFTKLLLKLPDLRSLNNMHSEELLAFKVH

SEQ ID NO: 9 - DNA, 20 bp, synthetic construct
  misc_feature 1..20: note = forward primer A for detection of E4bp4 wildtype allele
  source 1..20: mol_type = other DNA
ctctgagcttggctgatgtg

SEQ ID NO: 10 - DNA, 20 bp, synthetic construct
  misc_feature 1..20: note = reverse primer for the detection of E4bp4
  source 1..20: mol_type = other DNA
gcttcaagtctccaccaagc

SEQ ID NO: 11 - DNA, 20 bp, synthetic construct
  misc_feature 1..20: note = primer for the detection of the E4bp4 null allele
  source 1..20: mol_type = other DNA
ccatgctcctgtcttgatga

SEQ ID NO: 12 - protein, 8 aa, synthetic construct
  REGION 1..8: note = side chain on SUMO modified peptide
GGTQQQFV

SEQ ID NO: 13 - protein, 20 aa, Mus musculus
MQLRKMQTIKKEPAPLDPTS

SEQ ID NO: 14 - protein, 20 aa, Rattus rattus
MQLRKMQAIKKEPASLDPTG

SEQ ID NO: 15 - protein, 20 aa, Homo sapiens
MQLRKMQTVKKEQASLDASS

SEQ ID NO: 16 - protein, 20 aa, Gallus gallus
MQLRKMQTLKKEHGSVDTSS

SEQ ID NO: 17 - protein, 15 aa, Xenopus laevis
MPTIKKEQECADSRM

SEQ ID NO: 18 - protein, 20 aa, Mus musculus
LENKLIALGEENATLKAELL

SEQ ID NO: 19 - protein, 20 aa, Rattus rattus
LENKLIALGEENATLKAELL

SEQ ID NO: 20 - protein, 20 aa, Homo sapiens
LENKLIALGEENATLKAELL

SEQ ID NO: 21 - protein, 20 aa, Gallus gallus
LENKLIALGEENATLKAELL

SEQ ID NO: 22 - protein, 20 aa, Xenopus laevis
LENKLIALGEENASLKTELL

SEQ ID NO: 23 - protein, 20 aa, Mus musculus
PENKFPVIKQEPVELESFAR

SEQ ID NO: 24 - protein, 20 aa, Rattus rattus
PENKFPVIKQEPVELESFAR

SEQ ID NO: 25 - protein, 20 aa, Homo sapiens
PENKFQIIKQEPMELESYTR

SEQ ID NO: 26 - protein, 17 aa, Gallus gallus
PENKFQIIKQEPIELER

SEQ ID NO: 27 - protein, 20 aa, Xenopus laevis
TDIKSQRIKQEQMEAGNFSR

SEQ ID NO: 28 - protein, 20 aa, Mus musculus
RIKAKAMQVKVEALDSEFEG

SEQ ID NO: 29 - protein, 20 aa, Rattus rattus
RIKAKAMQVKVEALDSEFEG

SEQ ID NO: 30 - protein, 20 aa, Homo sapiens
RIKAKAMQIKVEAFDNEFEA

SEQ ID NO: 31 - protein, 20 aa, Gallus gallus
RIKAKAMQVKVEAMDNDYDA

SEQ ID NO: 32 - protein, 20 aa, Xenopus laevis
RIKAKAMQIKVESLESELNS

SEQ ID NO: 33 - protein, 20 aa, Mus musculus
VTNIQDWSLKSEHWHHKELS

SEQ ID NO: 34 - protein, 20 aa, Rattus rattus
VTNIQDWSLRSEHWHHKELG

SEQ ID NO: 35 - protein, 20 aa, Homo sapiens
VTNIQDWSLKSEHWHQKELS

SEQ ID NO: 36 - protein, 20 aa, Gallus gallus
VTNIQDWSLKPELWHQKELN

SEQ ID NO: 37 - protein, 20 aa, Xenopus laevis
VTNIQDWPLKPGQWHHRELE

SEQ ID NO: 38 - DNA, 27 bp, synthetic construct
  misc_feature 1..27: note = Forward primer for amplification of putative E4bp4-bindingregions in Notch1 locus
  source 1..27: mol_type = other DNA
ctatatttttgccttgacagctaaagg

SEQ ID NO: 39 - DNA, 20 bp, synthetic construct
  misc_feature 1..20: note = Reverse primer for amplification of putative E4bp4-bindingregions in Notch1 locus
  source 1..20: mol_type = other DNA
gaagtacgaagcatgcttgc

SEQ ID NO: 40 - DNA, 22 bp, synthetic construct
  misc_feature 1..22: note = Forward primer for amplification of putative E4bp4-bindingregions in Notch1 locus
  source 1..22: mol_type = other DNA
cacatctgtgagctatttttgg

SEQ ID NO: 41 - DNA, 24 bp, synthetic construct
  misc_feature 1..24: note = Reverse primer for amplification of putative E4bp4-bindingregions in Notch1 locus
  source 1..24: mol_type = other DNA
gactgactaaactaacattcccac

SEQ ID NO: 42 - DNA, 20 bp, synthetic construct
  misc_feature 1..20: note = Forward primer for amplification of putative E4bp4-bindingregions in Notch1 locus
  source 1..20: mol_type = other DNA
ctcagaaactggcctcaagc

SEQ ID NO: 43 - DNA, 19 bp, synthetic construct
  misc_feature 1..19: note = Reverse primer for amplification of putative E4bp4-bindingregions in Notch1 locus
  source 1..19: mol_type = other DNA
cacttgcagtcaggcgttc

SEQ ID NO: 44 - DNA, 20 bp, synthetic construct
  misc_feature 1..20: note = Forward primer for amplification of putative E4bp4-bindingregions in Notch1 locus
  source 1..20: mol_type = other DNA
cacgccatcttaaagagctc

SEQ ID NO: 45 - DNA, 22 bp, synthetic construct
  misc_feature 1..22: note = Reverse primer for amplification of putative E4bp4-bindingregions in Notch1 locus
  source 1..22: mol_type = other DNA
gtaaccaactgcactcttctcc

SEQ ID NO: 46 - DNA, 19 bp, synthetic construct
  misc_feature 1..19: note = Forward primer for amplification of putative E4bp4-bindingregions in Notch1 locus
  source 1..19: mol_type = other DNA
caccaagaattcccaggag

SEQ ID NO: 47 - DNA, 20 bp, synthetic construct
  misc_feature 1..20: note = Reverse primer for amplification of putative E4bp4-bindingregions in Notch1 locus
  source 1..20: mol_type = other DNA
gagtgcagtcacgtgctgac

SEQ ID NO: 48 - DNA, 23 bp, synthetic construct
  misc_feature 1..23: note = Forward primer for amplification of putative E4bp4-bindingregions in Notch1 locus
  source 1..23: mol_type = other DNA
ctcagactctctcggtaagtgtc

SEQ ID NO: 49 - DNA, 19 bp, synthetic construct
  misc_feature 1..19: note = Reverse primer for amplification of putative E4bp4-bindingregions in Notch1 locus
  source 1..19: mol_type = other DNA
cgtgtggagctactctggc

SEQ ID NO: 50 - DNA, 426 bp, Homo sapiens
  source 1..426: mol_type = unassigned DNA
atgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctttaa

SEQ ID NO: 51 - protein, 141 aa, Homo sapiens
MSNMRCVDCGTCLGHTRRHPTLFWGKTLPGLTPVAAPAPQPAQCPPGSEEDAPATQPGPQLAGPDPPWAPVFCRRLARAGHIELCNAVGCVLWSCPRSPGRGHAVGQGLEGGGGCPWATPPSLGGADFCNTKYSLWQKKCL

(19) United States
(12) Patent Application Publication
(10) Pub. No.: US 2023/0002732 A1
(43) Pub. Date: Jan. 5, 2023

(54) NATURAL KILLER CELLS

(71) Applicant: IMPERIAL COLLEGE INNOVATIONS LIMITED, London (GB)

(72) Inventors: Hugh J.M. Brady, London (GB); Matthew Fuchter, London (GB); Tomasz Kostrzewski, Welwyn (GB)

(73) Assignee: IMPERIAL COLLEGE INNOVATIONS LIMITED, London (GB)

(21) Appl. No.: 17/929,417

(22) Filed: Sep. 2, 2022

Related U.S. Application Data
(63) Continuation-in-part of application No. 16/497,669, filed on Sep. 25, 2019.

(30) Foreign Application Priority Data
Mar. 28, 2017 (GB) 1704953.7

(51) Int. Cl. C12N 5/0783; A61K 35/17

(52) U.S. Cl. CPC C12N 5/0646; A61K 35/17; C12N 2506/11; C12N 2501/42; C12N 2501/2303; C12N 2501/2306; C12N 2501/2307; C12N 2501/2315; C12N 2501/26; C12N 2501/125; C12N 2501/22; C12N 2501/145

(57) ABSTRACT

This invention relates to Natural Killer (NK) cell populations, to methods of producing the same and therapeutic applications thereof. More specifically, the invention relates to the expansion of NK cells by increasing the expression of specific transcription factors associated with NK cell production.

### SEQUENCE LISTING

A sequence listing in electronic (XML file) format is filed with this application and incorporated herein by reference. The name of the ASCII text file is “P48972US_C1_PCT.XML”; the file was created on Sep. 2, 2022; the size of the file is 63,862 bytes.

### FIELD OF THE INVENTION

This invention relates to expanded Natural Killer (NK) cell populations, to methods of producing the same and therapeutic applications thereof. More specifically, the invention relates to the expansion of NK cells by increasing the expression of specific transcription factors associated with NK cell production.

### BACKGROUND OF THE INVENTION

There has been an increase in interest in Natural Killer (NK) cells as they are cytotoxic against cancerous, pathogen-infected and otherwise damaged cells. NK cells are innate lymphoid cells (ILCs), specifically large granular cytotoxic lymphocytes that bridge the innate and the adaptive arms of the immune response. They make up 10-15% of circulating lymphocytes in the peripheral blood. NK cells also exhibit the highest level of cytotoxic activity within the immune system. Therefore, altered NK cell functionality or numbers impact the functioning of the immune system against infection and cancer. For example, a large scale study in Japan has shown that reduced levels of NK cells in a cohort of people aged over 40 is associated with a significantly higher incidence of cancer.

Similarly to B cells and T cells, these NK cells are derived from Common Lymphoid Progenitor (CLP) cells that in turn come from Haematopoietic Stem Cells (HSCs). However, NK cells are different from B and T cells as they lack specific cell surface antigen receptors. Due to this, NK cells may kill cancerous and pathogen-infected cells without prior sensitisation, making them part of the innate immune response. They also have a critical role in tumour immunosurveillance by directly influencing the adaptive immune response.

Activation of NK cells triggers them to release perforin and cytoplasmic granules containing granzymes. Perforin polymerises to form pores on target cells in the presence of Ca²⁺. Granzymes may enter these pores into target cells, causing DNA fragmentation and apoptosis. NK cells may also secrete cytokines, which trigger the action of other immune cells in the adaptive arm of the immunity.

Due to the importance of NK cells in immune response against pathogen infection and cancer cells, multiple clinical trials have tested the efficacy of NK cells in adoptive transfer protocols. In adoptive transfer, NK cells isolated from the blood of donors are expanded ex vivo and matured into healthy and functional NK cells prior to transfusion into recipients. However, to be effective it is crucial that NK cell donors are be screened for their KIR genotype, where the donor must have the appropriate KIR allelic polymorphism to the recipient to allow recognition of target cells for destruction. In any event, studies have found that the expanded products have lower clinical success rate than expected, with less ability to kill cancerous or infected cells. Thus, there are significant barriers to the current adoptive transfer protocols.

An alternative therapeutic approach is to increase the number of endogenous NK cells. One method is the administration of cytokines that are essential for NK cell development. Administration of IL-2 and IL-15 was predicted to enhance NK cell development. IL-2 promotes the proliferation and cytotoxicity of NK cells, whereas IL-15 promotes the development and expansion of NK cells. However, in in vivo studies, the cytokines were found only stimulate a minimal expansion of NK cells with reduced half-life, even at a very high dose. Further, administered cytokines often leads to systemic toxicity due to inappropriate activation of immune responses and the induction of NK cell apoptosis. In addition, even when previous methods have been able to increase NK cell expansion, the resulting expanded NK cells often exhibit limited functional activity, such as lower levels of IFNγ production and low levels of antibody-dependent cellular cytotoxicity (ADCC).

Thus, using conventional methods and techniques, producing large numbers of NK cells is difficult, and producing fully functional NK cells with high cytotoxicity is even harder. There is currently no drug available that selectively increases NK cell numbers. Therefore, there is a need to develop new methods of NK cell production; both ex vivo to produce large numbers of functional NK cells for therapeutic and research use; and in vivo.

### SUMMARY OF THE INVENTION

Natural Killer (NK) cells have a critical role in the immune system where they destroy cancerous, pathogen-infected or damaged cells. Boosting NK cell number or functionality is predicted to increase the killing of these cells. Existing therapies such as NK cell adoptive transfer and cytokine enhancement of endogenous NK cells are not very successful in terms of their efficacy.

NK cells are differentiated from the HSCs in the bone marrow and distributed throughout lymphoid and non-lymphoid tissues including lymph nodes, spleen, peripheral blood, lungs and liver. Specific cytokines and transcription factors are needed to encourage HSCs to develop into NK cells. Each cytokine and transcription factor must be present at a precise time and concentration in order to push differentiation from HSCs into NK cells. However, the precise hierarchy of cytokines and transcription factors governing NK cell maturation is still incompletely understood.

The present inventors have shown that abrogation of Notch signalling impedes NK cell production, and that the total lack of NK cell development from E4bp4^{-/-} progenitors can be completely rescued by short exposure to Notch peptide ligands, particularly Delta-like ligand 4 (DLL4). Based on this work, the inventors have developed a method for the ex vivo expansion of NK cells from haematopoietic progenitor cells (HPCs), which minimises exhaustion and produces large numbers of functional NK cells.

The present inventors have previously shown that inhibiting the action of REV-ERB increases NK cell production. In particular, the inventors demonstrated that inhibiting the action of REV-ERB (e.g. using the REV-ERB antagonist SR8278) increases E4bp4 expression, which in turn increases NK cell production. The inventors have now shown that combining these two independent mechanisms (use of Notch ligands and REV-ERB inhibition) results in a surprisingly potent means for enhancing NK cell production, allowing for the production of large numbers of functional NK cells that are suitable for in vivo therapeutic use more rapidly than the current methods.

In addition, the present inventors have also now shown that post-translational modification for E4bp4 can regulate E4bp4 activity. In particular, the inventors have shown that phosphorylation and SUMOylation decrease E4bp4 activity, and that, conversely decreasing phosphorylation and/or SUMOylation of E4bp4 can increase E4bp4 activity. Thus, post-transcriptional modification of E4bp4 may be used to increase E4bp4 activity, and hence increase NK cell production.

The inventors have now also developed a new method for the expansion of NK cells that surprisingly increases the proportion of CD16⁺ NK cells. In particular, the inventors have surprisingly found that a production method which includes a distinct pre-differentiation culture stage gives rise to NK cells with increased CD16 expression. Without being bound by theory, it is believed that inclusion of a prolonged pre-differentiation stage according to the invention affects the transcriptome of the CD34+ HPCs, resulting in epigenetic changes which favour CD16 expression in CD56+NK cells. As CD16⁺ NK cells are critical for ADCC, the method of the present invention advantageously allow for the production of expanded NK cell populations with improved functionality. As a further advantage, the invention allows for the production of CD16⁺ NK cells without the need to introduce exogenous genetic elements into the NK cells, which provide a further significant benefit when the NK cells are intended for clinical use. Thus, the ex vivo method for increasing the number of CD16⁺ NK cells in an expanded NK population, and expanded NK cell populations made by said method provide a significant advantage, enabling the expansion of NK cells from haematopoietic progenitor cells (HPCs), which minimises cellular exhaustion and produces large numbers of functional CD16⁺ NK cells.

Accordingly, the present invention provides an ex vivo method for expanding an NK cell population, comprising the steps of: a) culturing an haematopoietic progenitor cell (HPC) comprising sample obtained from an individual with a compound that inhibits the action of REV-ERB; b) culturing said cells in the presence of a Notch ligand; and c) expanding said cells in vitro to produce an NK cell population. In some embodiments, the vessel in which the HPCs are cultured is coated with the Notch ligand. The Notch ligand may be delta-like ligand 4 (DLL4), or a fragment thereof which retains the function of DLL4, or a molecule which mimics the function/activity of DLL4. Said compound may increase E4bp4 expression by decreasing REV-ERB activity. In some embodiments, said compound decreases the activity of REV-ERB-α and/or REV-ERB-β, preferably REV-ERB-β. In some embodiments, said compound preferably decreases the activity of REV-ERB-α and REV-ERB-β. Said compound may be a REV-ERB antagonist, preferably an antagonist of REV-ERB-α and REV-ERB-β. Said compound may be selected from a small molecule, a PROTAC reagent, a double stranded RNA (dsRNA), a small interfering RNA (siRNA), a small hairpin RNA (shRNA), a micro RNA, an antisense RNA, an aptamer, an antibody, a ribozyme, a peptide or a peptidomimetic. In some embodiments, preferably the compound is a small molecule. The compound may be SR8278, ARN5187, ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine or 1-(4-fluorophenyl)-N-(3-((1-methylpiperidin-4-yl)methyl)benzyl)cyclopentan-1-amine, preferably SR8278. The compound may be added no later than 2 days after isolating the HPCs in the sample, and optionally the Notch ligand is present on or from 4 days after isolating said HPCs.

The invention further provides an ex vivo method for expanding an Natural Killer (NK) cell population, comprising: a) culturing an haematopoietic progenitor cell (HPC) comprising sample obtained from an individual in the presence of a delta-like ligand 4 (DLL4), or a fragment thereof which retains the function of DLL4, or a molecule which mimics the activity/function of DLL4; and b) culturing the cells produced by step (a) in the presence of IL-15; thereby producing an expanded NK cell population. The vessel in which the HPCs are cultured in step (a) may be coated with the DLL4 ligand or fragment thereof. In some embodiments, in step (a) the HPCs are also cultured in the presence of IL-7, Flt3L and/or stem cell factor (SCF), preferably IL-7, Flt3L and SCF. Step (a) and/or step (b) may be carried out in the absence of a stromal support cell, in some preferred embodiments both step (a) and step (b) are carried out in the absence of a stromal support cell.

The sample of HPCs is obtained from bone marrow, cord blood and/or peripheral blood.

The invention also provides an expanded NK cell population obtained by the method of any the invention, wherein at least 85% of the NK cells are CD56+ and CD45+.

The invention further provides a composition comprising an expanded NK cell population of the invention and a pharmaceutically acceptable carrier, diluent and/or excipient.

The invention further provides products containing a compound which inhibits the action of REV-ERB and a Notch ligand as a combined preparation for simultaneous, separate or sequential use in a method of therapy by increasing production of natural killer (NK) cells in a patient. In some embodiments: a) said compound is a compound as defined herein; and/or b) said Notch ligand is delta-like ligand 4 (DLL4), a fragment thereof which retains the function of DLL4, or a molecule which mimics the function/activity of DLL4. Said method of therapy may be a method of treating a disease or disorder selected from cancer, an infectious disease (acute or chronic), an autoimmune disease or a disease or disorder related to female infertility or pregnancy. Said method of therapy may be a method of treatment of a viral infection, a bacterial infection, a protest infection, a fungal infection and/or a helminth infection. The productions for use of the invention may be used in combination with antibody-mediated immunotherapy. In some embodiments of the products for use of the invention, said compound and/or Notch ligand is for administration before, simultaneously with, or after administration of the antibody-mediated immunotherapy. Said compound may be SR8278, ARN5187, ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine or 1-(4-fluorophenyl)-N-(3-((1-methylpiperidin-4-yl)methyl)benzyl)cyclopentan-1-amine, preferably SR8278.

The invention also provides a method of treatment by increasing the number of NK cells in a patient in need thereof, comprising administering to said patient a therapeutically effective amount of a compound which inhibits the action of REV-ERB (as defined herein) and a Notch ligand. The Notch ligand may be delta-like ligand 4 (DLL4), or a fragment thereof which retains the function of DLL4. In such methods, the compound and Notch ligand may be used in combination with antibody-mediated immunotherapy.

The invention further provides an ex vivo method for expanding an NK cell population, comprising the steps of: a) culturing an HPC comprising sample obtained from an individual; b) contacting said sample with a compound which results in the post-translational modification of E4bp4, thereby causing an increase in E4bp4 activity; and c) expanding said cells in vitro to produce an NK cell population. Said post-translation modification of E4bp4 is typically a reduction in SUMOylation and/or phosphorylation of E4bp4. In some embodiments, the compound: a) reduces SUMOylation at one or more of residues K10, K116, K219, K337 and/or K394 of E4bp4, or a residue corresponding thereto, or any combination thereof; and/or b) reduces phosphorylation at one or more of residues S286, S301 and/or S454, or a residue corresponding thereto, or any combination thereof.

The present invention also provides an ex vivo method for producing an expanded population of CD16+ Natural Killer (NK) cells, comprising the steps of: (A) culturing an haematopoietic progenitor cell (HPC) comprising sample obtained from an individual in medium which does not induce differentiation of the HPCs, wherein the culturing is for between about 2 to about 8 days, to produce a pre-differentiation HPC population; and (B) culturing the pre-differentiation HPC population in medium which induces differentiation of the HPCs to NK.

The invention also provides an ex vivo method for increasing the number of CD16+NK cells in an expanded NK cell population, comprising the steps of: (A) culturing an haematopoietic progenitor cell (HPC) comprising sample obtained from an individual in medium which does not induce differentiation of the HPCs, wherein the culturing is for between about 2 to about 8 days, to produce a pre-differentiation HPC population; and (B) culturing the pre-differentiation HPC population in medium which induces differentiation of the HPCs to NK.

In step (A) the HPCs may be cultured for between about 2 days to about 6 days, optionally for between about 4 days to about 6 days.

The HPCs may be cultured in the presence of a Notch ligand for at least part of step (A).

The HPCs may be cultured in a vessel that is coated with the Notch ligand for at least part of step (A). In each instance of culturing, the Notch ligand may be delta-like ligand 4 (DLL4), or a fragment thereof which retains the function of DLL4. Preferably in step (A) the HPCs are cultured in the absence of the Notch ligand for about 1 day followed by culture in the presence of the Notch ligand for the remainder of step (A).

In step (A) the HPCs may be cultured in the presence of a compound which inhibits the action of REV-ERB; and/or in step (B) the pre-differentiation HPC population may be cultured in the presence of a compound which inhibits the action of REV-ERB.

Said compound may: (i) increase E4bp4 expression by decreasing REV-ERB activity; (ii) decrease the activity of REV-ERB-α and/or REV-ERB-β, preferably REV-ERB-β; (iii) decrease the activity of REV-ERB-α and REV-ERB-β; (iv) be a REV-ERB antagonist, preferably an antagonist of REV-ERB-α and REV-ERB-β; (v) be selected from a small molecule, a PROTAC reagent, a double stranded RNA (dsRNA), a small interfering RNA (siRNA), a small hairpin RNA (shRNA), a micro RNA, an antisense RNA, an aptamer, an antibody, a ribozyme, a peptide or a peptidomimetic, preferably a small molecule; and/or (vi) be SR8278,

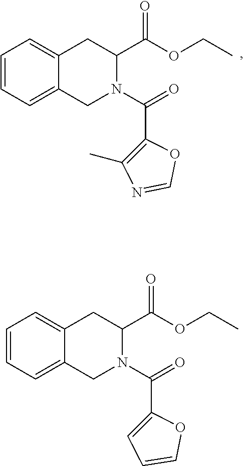 [SMILES: CCOC(=O)C1Cc2ccccc2CN1C(=O)c1ocnc1C ; CCOC(=O)C1Cc2ccccc2CN1C(=O)c1ccco1]

### or GSK1362.

The medium which does not induces differentiation of the HPCs in step (A) and/or the medium which induces differentiation of the HPCs to NK in step (B) may not comprise IL-3, preferably wherein the medium which induces differentiation of the HPCs to NK in step (B) does not comprise IL-3.

The medium in step (A) may comprise at least one of Flt3L, GM-CSF, IL-3, IL-6, TPO and/or stem cell factor (SCF), preferably Flt3L, GM-CSF, IL-3, IL-6, TPO and SCF. The medium in step (B) may comprise IL-7, Flt3L, IL-1S, and/or SCF, preferably IL-7, Flt3L, IL-15 and SCF.

Step (A) and/or step (B) may be carried out in the absence of a stromal support cell, preferably wherein both step (A) and step (B) are carried out in the absence of a stromal support cell.

The sample of HPCs may be obtained from bone marrow, cord blood and/or peripheral blood.

The proportion of CD16+NK cells may be increased compared with the proportion of CD16+NK cells produced by a corresponding method in which step (A) is omitted. The expanded NK cell population may comprise at least 10% CD16+NK cells, preferably at least 15% CD16+NK cells, more preferably at least 20% CD16+NK cells, even more preferably at least 30% CD16+NK cells. The expanded NK cell population may exhibit at least 30% greater antibody-dependent cellular cytotoxicity (ADCC), preferably at least 50% greater ADCC, compared with NK cells produced by a corresponding method in which step (A) is omitted.

In certain embodiments of this method, the method excludes a (further) step of introducing exogenous nucleic acid into the HPCs and/or NK cells.

The invention further provides an expanded population of CD16+NK cells, wherein at least 10% of the NK cells are CD16+NK cells, preferably at least 15% of the NK cells are CD16+NK cells, more preferably at least 20% of the NK cells are CD16+NK cells, even more preferably at least 30% of the NK cells are CD16+NK cells.

The invention provides an expanded population of CD16+NK cells obtained by the method of the invention, wherein at least 10% of the NK cells are CD16+NK cells, preferably at least 15% of the NK cells are CD16+NK cells, more preferably at least 20% of the NK cells are CD16+NK cells, even more preferably at least 30% of the NK cells are CD16+NK cells.

In certain embodiments of this method, the CD16+NK cells may not comprise exogenous nucleic acid.

The expanded NK cell population may exhibit at least 30% greater ADCC, preferably at least 50% greater ADCC, more preferably at least 70% greater ADCC, compared with NK cells produced by a corresponding method in which step (A) is omitted.

The invention further provides a composition comprising an expanded NK cell of the invention and a pharmaceutically acceptable carrier, diluent and/or excipient.

The invention also provides an expanded population of CD16+NK cells or a composition comprising such cells of the invention for use in a method of therapy.

Said method of therapy may be a method of treating a disease or disorder selected from cancer, an infectious disease (acute or chronic), an autoimmune disease or a disease or disorder related to female infertility or pregnancy. Said method of therapy may be a method of treatment of a viral infection, a bacterial infection, a protist infection, a fungal infection and/or a helminth infection.

The invention also provides an expanded population of CD16+NK cells or composition comprising such cells for use according to the invention, which is used in combination with antibody-mediated immunotherapy. Said composition may be for administration before, simultaneously with, or after administration of the antibody-mediated immunotherapy.

### BRIEF DESCRIPTION OF THE DRAWINGS

FIG. 1: NK cell developmental pathway. NK cells are differentiated from Hematopoietic Stem Cells (HSCs). NK cells develop from HSC into Common Lymphoid Progenitor (CLP) cells, NK progenitor (NKP) cells, immature NK (iNK) cells, mature NK (mNK) cells and finally into conventional NK (cNK) cells, which circulate in the bloodstream. Below the diagram of the pathway are the cytokines and transcription factors that are required for NK cell development. IL-15 is one of the main cytokine required for the development of NK cells. Others are transcription factors required for the transitions shown on the diagram.

FIG. 2: (A) E4bp4 was expressed in HeLa cells stably expressing 6His-SUMO1, 6His-SUMO2 or 6His-SUMO3. Protein extracts were purified by Ni²⁺ affinity chromatography under denaturing conditions and analysed by Western blot. Input samples were lysed in Laemlli sample buffer and directly compared. (B) FLAG-E4bp4 was expressed in 6His-SUMO1, 6His-SUMO2 and 6His-SUMO3 HeLa cells and anti-FLAG antibody was used to immunoprecipitate E4bp4 before Western blot analysis. Grey arrows indicate unmodified E4bp4 and black arrows indicate SUMO modified forms of E4bp4 with higher molecular weights. Both are representative of three independent experiments.

FIG. 3: (A) Sites of potential E4bp4 SUMO modification based on the presence of the 4ψ-K-x-E motif. Asterisks highlight perfectly conserved residues and dots highlight partially conserved residues. Position of last amino acid in sequence indicated. (B) Mutant versions of E4bp4 lacking SUMOylation sites (lysine to arginine point mutations) were expressed in 6His-SUMO2 HeLa cells and protein extracts were purified by Ni²⁺ affinity chromatography under denaturing conditions and analysed by Western blots. Grey arrows highlight unmodified E4bp4 and black arrows highlight SUMO modified forms of E4bp4 with higher molecular weights, representative of three independent experiments. (C) Mass spectrometry identification of E4bp4 peptide SUMO2/3 modified at K219. FLAG-E4bp4 was expressed in 293T cells, purified from whole cell lysate using anti-FLAG immunoprecipitation and subjected to sequential digest by trypsin and Glu-C. E4bp4 peptides were purified and analysed by LC-MS/MS and SUMOylated peptides were identified by the presence of a GGTQQQFV modification on a lysine side chain. Annotated CID tandem mass spectra of +2 ion at m/z 853.95, with schematic representation of the identified peptide shown with detected b and γ ions labelled from the fragmentation of E4bp4 peptide and SUMO tag.

FIG. 4: Lin⁻ BM cells were isolated from E4bp4^{-/-} (A) or E4bp4+/+(C) mouse BM and cultured for two days in the presence of IL-7, Flt3-L and SCF before retroviral transduction with a MSCV-IRES-hCD2 construct. Transduced cells were cultured for three days, before being transferred onto OP9 stromal cells and cultured in the presence of IL-15 for 7 days. (A, C) Flow cytometry analysis identified hCD2⁺ (transduced) cells and the presence of NK1.1⁺ CD19⁻ NK cells. (B, D) Relative levels of NK cell production between E4bp4 mutants normalised to WT-form E4bp4 (B) or empty vector (D). Data are representative of four independent experiments for each mutant. Error bars show SEM. *, P<0.05.

FIG. 5: (A) Schematic representation of the E4bp4 protein showing the positions of phosphorylation sites identified by LC-MS/MS. Conserved domain structure of E4bp4 shown (not to scale), which has been identified through sequence homology and mutational studies 50. (B, D) Lin-BM cells were isolated from E4bp4^{-/-} (B) or E4bp4^{+/+} (D) mouse BM and cultured for two days in the presence of IL-7, Flt3-L and SCF before retroviral transduction with a MSCV-IRES-hCD2 construct. Transduced cells were cultured for three days, before being transferred onto OP9 stromal cells and cultured in the presence of IL-15 for 7 days. Flow cytometry analysis identified hCD2⁺ (transduced) cells and the presence of NK1.1⁺ CD19⁻ NK cells. (C, E) Relative levels of NK cell production between E4bp4 mutants normalised to WT-form E4bp4 (C) or empty vector (E). Data are representative of three independent experiments for each mutant. Error bars show SEM. *, P<0.05.

FIG. 6: Annotated CID tandem mass spectrum of the +2 ion at m/z 699.77, confirming phosphorylation of E4bp4 at S286. Tandem mass spectra acquired with an electrospray ionization LTQ/Orbitrap mass spectrometer from purified FLAG-E4bp4. FLAG-E4bp4 was expressed in HEK-293T cells and purified by immunoprecipitation with anti-FLAG M2 affinity resin and the purified protein competitively eluted using 3× FLAG peptide. Purified E4bp4 was digested with trypsin and subjected to phosphopeptide enrichment using TiO₂ beads to reduce background from unphosphorylated E4bp4 peptides. Magnification of certain regions of the spectra is highlighted. Spectra is schematic representation of fragmented peptide with identified ions labelled.

FIG. 7: Annotated CID tandem mass spectrum of the +2 ion at m/z 656.83, confirming phosphorylation of E4bp4 at S301. Details of method as above for FIG. 6.

FIG. 8: Annotated CID tandem mass spectrum of the +2 ion at m/z 526.76, confirming phosphorylation of E4bp4 at S301. Details of method as above for FIG. 6.

FIG. 9: (A) 3T3 cells stably expressing E4bp4, 5×-SUMO and S286-353A were treated with cycloheximide (CHX, 50 μg ml⁻¹) for the times indicated. Western blotting was used to compare nuclear extracts. (B) Quantification of relative E4bp4 expression from Western blot signals using densitometry. Level of E4bp4 is represented in arbitrary units after signals were normalised relative to those of Histone H3. (C, D) Transcriptional reporter assay using a Firefly Luciferase gene downstream of E4bp4 consensus binding sites. Relative luciferase activity was measured after 48 h from 3T3 cells transfected with MSCV-E4bp4-IRES-hCD2 and Firefly luciferase construct. Data are representative of ten independent experiments for each mutant. (E) MNK-1 cells were transduced with either vector control, E4bp4, 5×-SUMO or S286-353A. Expression of Eomes, Gata3, Tbet and Notch1 were determined by QPCR. Data are representative of three independent experiments for each mutant. Error bars show SEM. *, P<0.05.

FIG. 10: Notch1 is a transcriptional target of E4bp4 in NK cells. (A) Notch1 locus showing location of predicted E4bp4 binding sites identified through the presence of the E4bp4 minimal consensus binding sequence TTA(T/C)(G/A)TAA(C/T). Filled boxes=exons; clear boxes=UTR regions (B) ChIP analysis of E4bp4 binding at the Notch1 loci in chromatin from MNK-1 cells stably transduced with FLAG-E4bp4. E16 is a polyclonal antibody to E4bp4 and M2 is a monoclonal antibody to FLAG. Per2B was used as a positive control and gene desert as a negative control. Data is representative of three biological replicates. Error bars show SEM. *, P<0.05. (C) QPCR analysis of Notch1 expression in Lin⁻ BM cells. Data is representative of six biological replicates. Error bars show SEM. *, P<0.05.

FIG. 11: Notch signalling at an early stage can induce NK cell development in the absence of E4bp4. (A) Schematic representation of culture system used to induce Notch signalling and promote NK cell development from Lin⁻ BM cells. (B) Flow cytometry analysis of NK cell production following cultivation on indicated combination of OP9 or OP9-DL1 stromal cells or (C) on empty plates or plates coated with recombinant rDLL1/rDLL4. Lin⁻ BM cells were first cultured for 7 days in IL-7, Flt3L, SCF before transferring to new plates for culture in IL-15. Data is representative of four (B) or two (C) biological replicates. (D) Schematic representation of two-stage culture of NK cell development from Lin⁻ BM cells following from Cre-mediated deletion of Rbpj gene following lentiviral transduction. (E) Flow cytometry analysis of NK cell production derived from Lin⁻ BM isolated from Rbpj floxed mice and transduced with either empty or Cre-expressing lentiviral vector. Cells were cultivated either on OP9 stromal cells alone or on rDLL4-coated plates before transfer on to OP9 as indicated. Data shown is for cells gated on hCD2 expression.

FIG. 12: Synergy between REV-ERB antagonism and the Notch ligand, rDLL4. (A) Flow cytometry analysis of NK cell production following cultivation with the indicated combinations of SR8278 and rDLL4. (B) The graph shows the percentage of mature (NK1.1⁺NKp46⁺) NK cells against the different conditions.

FIG. 13: Experimental design to test if there is a synergistic effect of the REV-ERB antagonist SR8278 with the recombinant Notch ligand rDLL4 on NK cell production.

FIG. 14: Synergy between REV-ERB antagonism and the Notch ligand, rDLL4. The graph shows the percentage of mature (NK1.1⁺NKp46⁺) NK cells against the various conditions (as in FIG. 13). Condition f shows the strongest effect. Error bars show SEM. *, P50.05; **, P50.01; ***, P50.001; ****, P50.0001. These data represent technical triplicates of biological duplicates.

FIG. 15: Exposure to Notch ligand greatly accelerates the production of phenotypically mature human NK cells from human CD34⁺ umbilical cord blood. The graph shows the percentage of mature (CD46⁺/CD56⁺) NK cells against time for human CD34⁺ cells cultured under control condition (light grey bars) or on rDLL4 (dark grey bars).

FIG. 16: Experimental timelines and flow cytometry data demonstrating that addition of DLL4 further increases CD16 expression when combined with a set pre-differentiation step.

FIG. 17A: Graphs showing % and absolute number of CD56⁺ NK cells over time in the presence or absence of DLL4 with set pre-differentiation steps of different lengths. •=+DLL4; ▴=no DLL4

FIG. 17B: Graphs showing % and absolute number of CD56⁺CD16⁺ NK cells over time in the presence or absence of DLL4 with set pre-differentiation steps of different lengths. •=+DLL4; ▴=no DLL4

FIG. 18: Graph showing the effect of IL-3 on the % of CD56⁺CD16⁺ NK cells over time when HPCs are cultured in the presence/absence of DLL4 and/or presence/absence of EL08 stromal cells.

### DETAILED DESCRIPTION OF THE INVENTION

### Natural Killer Cells

Natural Killer (NK) cells exhibit the highest level of cytotoxic activity within the immune system. NK cells are similar to B cells and T cells, but lack specific cell surface antigen receptors. Instead, NK cells have activatory and inhibitory receptors that recognise motifs.

NK cells circulate in the blood and the peripheral lymphoid organs such as lymph nodes and spleen. They can become activated by cytokines or upon encountering target cells. The recognition and elimination of target cells is based on balancing between inhibitory and activatory signals. Activatory signals are generated by activatory receptors (NKG2D, NKp46, NKp30) binding to ligands, which can be present not only on cancerous, pathogen-infected and damaged cells, but also on healthy cells. On the other hand, inhibitory signals are generated when inhibitory receptors (KIR, CD94/NKG2A) on NK cells bind to Major Histocompatability Complex (MHC) Class I molecules that are normally present on all healthy cells. MHC Class I molecules on target cells are absent or greatly downregulated, making them ideal NK cell targets. This allowed NK cells to distinguish between target and healthy cells. In order for NK cells to recognise and kill target cells, overall activatory signals must be greater than inhibitory signals.

NK cells recognise and kill cancerous, pathogen-infected and damaged cells without prior sensitisation, making them part of the innate immune response. For example, NK cells provide an early response to virus infection, occurring prior to T cell killing of infected cells. NK cells can kill target cells within minutes. NK cells also secrete cytokines and “weaponise” other parts of the immune system. For example, NK cells promote T cell effector function and enhance antibody-directed cellular cytotoxicity (ADCC).

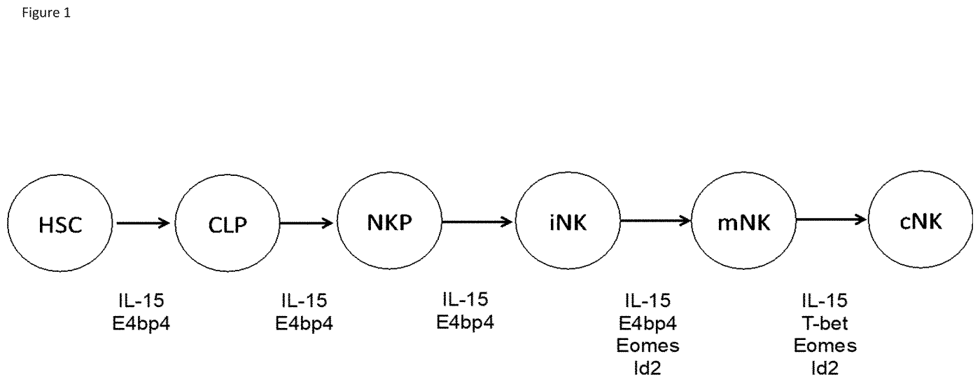

NK cells are differentiated from haematopoietic stem cells (HSCs) via the pathway set out in FIG. 1. In more detail, NK cells develop from HSCs into Common Lymphoid Progenitor (CLP) cells, pre-NK progenitor (pre-NKP) cells, NK progenitor (NKP) cells, immature NK (iNK) cells, mature NK (mNK) cells and finally into conventional NK (cNK) cells, which circulate in the bloodstream. Although this terminology derives from NK cell development in mice, a corresponding pathway occurs in human NK cell development. For example, HSCs develop through multiple stages of precursors (stage 1, 2 and 3), before developing into mature NK cells (stages 4 and 5). For consistency, references HSCs, CLPs, pre-NKPs, NKPs, iNK, mNK, cNK and NK cells are used herein. However, in the context of the present invention, these terms are interchangeable with stages 1 to 5 of the human nomenclature. Below the diagram of the pathway in FIG. 1 are the cytokines and transcription factors that are essential for NK cell development. IL-15 is one of the main cytokine required for the development of NK cells. Other extrinsic factors, such as specific stromal cells, are also required for the development and maturation of NK cells. According to the present invention, Hematopoietic Progenitor Cells (HPCs) are a heterogeneous population containing multi-potential progenitors such as HSCs, CLPs and also NKPs. HPCs are referred to as lineage negative cells, as they have not yet committed to a developmental pathway. Accordingly, in the context of the present invention, HSCs, CLP cells and NKP cells are all HPCs and a reference to HPCs is a reference to any of HSCs, CLP cells and/or CLP cells, or any combination thereof, unless explicitly stated to the contrary.

Due to the importance of NK cells in immune response, multiple clinical trials have tested the efficacy of NK cells in adoptive transfer protocols. Typically this is allogenic transfer, with the NK cells being isolated from a healthy donor and expanded. However, the downregulation of MHC Class I molecules on target cells is partial and the KIR genotype from donors and recipients may be similar. Due to this, NK cells transfused into recipients, even from different individuals may not attach target cells if their KIRs recognise MHC Class I molecules. Therefore, it is crucial that NK cell donors must be screened for their KIR genotype, where the donor must have the appropriate KIR allelic polymorphism to the recipient to allow recognition of target cells for destruction. Moreover, the expanded products were found to have lower clinical success rate than expected, with less ability to kill cancerous or infected cells.

An NK cell may be defined in terms of its marker expression, its function/activity, or a combination thereof. Such definitions are standard in the art and methods are known by which marker expression and/or NK cell activity may be assessed. Thus, one of skill in the art would readily be able to categorise a cell as an NK cell using standard methodology and definitions.

For example, mNK and cNK cells may be recognised by their expression of the surface markers CD16 (FcγRIII) and/or CD56, typically both CD16 and CD56 in humans, and NK1.1 or NK1.2 in some mice strains. NKp46 is another marker for mNK and cNK cells, and is expressed in humans and several mice strains. Thus, NKp46 may be used as a marker for NK cells either with or without CD16 and/or CD56 (in humans) or with or without NK1.1 or NK1.2 (in mice). Other examples of makers which can be used to identify/define NK cells according to the present invention include Ly49, natural cytotoxicity receptors (NCRs), CD94, NKG2, killer-cell immunoglobulin-like receptors (KIRs), and/or leukocyte inhibitory receptors (ILT or LIR), or any combination thereof, including in combination with CD16 and or CD56 (in humans) or NK1.1/NK1.2 (in mice). In some preferred embodiments mature NK cells according to the invention (i.e. mNK and cNK cells) are CD56⁺ and CD45⁺, and may be also be CD16⁺. As used herein, the term mature human NK cell encompasses NK cells that are CD56^{bright} (stage 4) and CD56^{dim} (stage 5), both of which are CD56⁺. Mature NK cells may also be defined by the absence of markers, such as CD34, and lymphocyte markers CD3 and/or CD19. Thus, mature NK cells of the invention may be CD56⁺, CD45⁺, CD16, CD3⁻ and/or CD19⁻, or any combination thereof, such as CD56⁺, CD45⁺, CD16⁺, CD3⁻ and CD19⁻.

In addition or alternatively, an NK may be identified by/defined in terms of its activity. For example, an NK cell may be identified/defined by the presence of cytolytic granules within its cytoplasm, by its ability to secrete antimicrobial molecules such as α-defensins, and/or its ability to secrete cytokines such as TNF-α, IL-10, IFN-γ and TFG-β.

Unless otherwise stated herein, a reference to NK cells includes a reference to iNK, mNK and cNK cells. HSCs, CLP cells and NKPs will typically be referred to as such.

### CD16 and Antibody-Dependent Cellular Cytotoxicity (ADCC)

CD16 (FcγRIII) is an Fc receptor expressed on the surface of NK cells, neutrophils, monocytes, macrophages, and some T cells. CD16A (FcγRIIIA) is expressed by NK cells. The human CD16A protein (UniProt Accession No. P08637, sequence version 2, accessed 26 May 2022) is expressed on NK cells. The terms CD16, CD16A, FcγRIII and FcγRIIIA are used interchangeably herein.

CD16 signals through association with the Fc receptor common γ chain, which possesses immunoreceptor tyrosine-based activation motifs (ITAMs). Signalling through CD16 mediates ADCC by NK cells. In ADCC the Fc regions of antibodies bound to the surface of target cells are recognised and bound by CD16 on the surface of NK cells. Cross-linking of CD16 with the antibody Fc regions activates the NK cells, resulting in the release of cytotoxic factors by the NK cells (also known as degranulation) that cause the death of the target cell by apoptosis. Therefore, NK cells with increased CD16 expression exhibit increased ADCC compared with NK cells with no or low CD16 expression.

The present inventors have developed an ex vivo method for expanding NK cells which also increases the number of CD16⁺ NK cells within the resulting expanding NK cell population.

Therefore, the invention provides mature NK cells, and particular expanded populations of NK cells (typically mature NK cells) which are CD56⁺, CD45⁺, CD3⁻ and/or CD19⁻ (e.g. CD56⁺, CD45⁺, CD3⁻ and CD19⁻) and which also have increased CD16⁺ expression compared with a control expanded NK cell population. A control expanded NK cell population may be one that is produced by any conventional method for NK cell expansion, or a corresponding method to that described herein, but without the pre-differentiation step (step (A)).

Therefore, the invention provides an expanded population of NK cells, wherein at least 10% of the NK cells in the population are CD16⁺ NK cells, at least 15% of the NK cells in the population are CD16⁺ NK cells, at least 20% of the NK cells in the population are CD16⁺ NK cells, at least 25% of the NK cells in the population are CD16⁺ NK cells, at least 30% of the NK cells in the population are CD16⁺ NK cells, at least 35% of the NK cells in the population are CD16⁺ NK cells, at least 40% of the NK cells in the population are CD16⁺ NK cells, at least 45% of the NK cells in the population are CD16⁺ NK cells, at least 50% of the NK cells in the population are CD16⁺ NK cells, at least 60% of the NK cells in the population are CD16⁺ NK cells, at least 70% of the NK cells in the population are CD16⁺ NK cells, at least 80% of the NK cells in the population are CD16⁺ NK cells, up to 100% of the NK cells in the population are CD16⁺ NK cells. Preferably at least 15% of the NK cells are CD16⁺ NK cells, more preferably at least 20% of the NK cells are CD16⁺ NK cells, still more preferably at least 25% of the NK cells are CD16⁺ NK cells even more preferably at least 30% of the NK cells are CD16⁺ NK cells, even more preferably at least 40% of the NK cells are CD16⁺ NK cells. Such expanded NK cell populations can typically be obtained by a method of the invention. CD16 expression may be determined using standard techniques, examples of which are known in the art and could be routinely selected by one of ordinary skill. Non-limiting examples of suitable techniques include flow cytometry, cell imaging and ELISA.

One benefit of the CD16⁺ NK cells of the invention is that they exhibit increased CD16 expression without the need for introduction of exogenous nucleic acid (such as by transduction or transfection). This is in contrast to conventional methods for expanding NK cells with increased CD16 expression, which require expression of an exogenous CD16 transgene to achieve even minimal increases in CD16 expression. Alternatively or in addition, another benefit of the CD16⁺ NK cells of the invention is that an NK cell population with an increased proportion CD16⁺ NK cells can be obtained with no or reduced purification and/or concentration compared with conventionally produced expanded NK cell populations. As such, conventionally produced expanded NK cell populations differ from those according to the invention, even if NK cells within said populations are manipulated to increase CD16 expression, and/or said conventionally produced NK cell population is manipulated to select and/or concentrate CD16⁺ NK cells, because (i) the CD16⁺ NK cells within such conventional populations will have been genetically altered and comprise exogenous nucleic acid, which is associated with disadvantages from a clinical/GMP perspective; and/or (ii) when extensive purification and/or concentration of CD16⁺ NK cells is required, this can result in damage to the purified and/or concentrated NK cells.

Accordingly, the invention provides a population of NK cells comprising an increased number of CD16⁺ NK cells (also referred to herein as a CD16⁺NK cell population), wherein the CD16⁺ NK cells have not been contacted with an exogenous nucleic acid (typically a nucleic acid encoding for CD16). Typically, a population of NK cells comprising an increased number of CD16⁺ NK cells according to the invention may comprise CD16⁺ NK cells that do not comprise exogenous nucleic acid encoding for CD16. In view of the potential clinical application of the NK cells of the invention, a population of NK cells comprising an increased number of CD16⁺ NK cells according to the invention may comprise CD16⁺ NK cells that do not comprise any exogenous nucleic acid. The term “exogenous nucleic acid” encompasses a naked nucleic acid (e.g. a plasmid), or a viral vector (e.g. AAV or lentiviral vector) used to introduce a nucleic acid of interest (e.g. a nucleic acid encoding CD16) into NK cells. Alternatively or in addition, the invention provides a population of NK cells comprising an increased number of CD16⁺ NK cells (also referred to herein as a CD16⁺ NK cell population), wherein the CD16⁺ NK cells have been subjected to no or reduced purification and/or concentration compared to CD16⁺ NK cells that have been produced by conventional means, including means comprising the use of exogenous nucleic acid encoding for CD16.

As described herein, CD16⁺ NK cells are more effective at mediating ADCC, as it is binding of CD16 to the Fc portion of antibodies that triggers degranulation of NK cells and hence ADCC. Accordingly, by providing expanded NK cell populations with increased numbers of CD16⁺ NK cells, the invention provides CD16⁺ NK cells and expanded NK cell populations which exhibit increased ADCC, i.e. increased ADCC activity. This increase in ADCC can be compared with a suitable reference or control NK cell or NK cell population. A control method may be any standard method known in the art for producing NK cell populations. For example, a control method may use conventional adoptive transfer techniques, rather than a method according to the present invention. One example of suitable control NK cells or NK cell populations is an NK cell or NK cell population produced by a corresponding method in which the HPCs are not cultured in a medium which does not induce differentiation of the HPCs to NK cells, or wherein the HPCs are cultured in a medium which does not induce differentiation of the HPCs to NK cells for less than 2 days. In other words, one example of suitable control NK cells or NK cell populations is an NK cell or NK cell population produced by a corresponding method in which step (A) as described herein is omitted. NK cells and NK cell populations produced by such control/standard methods may be used as control cells and populations as described herein.

An expanded population of NK cells according to the invention or the CD16⁺ NK cells within said population may exhibit at least 25% greater ADCC, such as at least 30% greater ADCC, at least 40% greater ADCC, at least 50% greater ADCC, at least 60% greater ADCC at least 70% greater ADCC, at least 80% greater ADCC, preferably at least 50% greater ADCC, more preferably at least 60% greater ADCC, still more preferably at least 70% greater ADCC, compared with a suitable reference or control NK cell or NK cell population.

### Expanded NK Cell Populations

As disclosed herein, the invention provides methods for generating an expanded population of NK cells (referred to interchangeably herein as an expanded NK cell population or an NK cell population). Also as disclosed herein, the invention provides methods for generating an expanded population of NK cells comprising an increased number of CD16⁺ NK cells (referred to interchangeably herein as an expanded CD16⁺NK cell population, a CD16⁺ NK cell population, an NK cell population or an expanded NK cell population of the invention). Any of the disclosure herein in relation to NK cells of the present invention may also be applied to an expanded NK cell population of the invention. Similarly, any of the disclosure herein in relation to NK cells and expanded NK cell populations of the invention applies equally and without reservation to CD16⁺ NK cells and expanded CD16⁺ NK cell populations.

Accordingly, the present invention provides an expanded NK cell population, such as a CD16⁺ NK cell population. Typically an expanded NK cell population of the invention comprises iNK cells, mNK cells and/or cNK cells, or a combination thereof. Said population may comprise HPCs, such as HSCs, CLP cells and/or NKPs, or a combination thereof, although the numbers of such cells is typically low relative to the number of NK cells, as the majority of these HPCs have differentiated into NK cells in the population. Said population may comprise other immune and/or non-immune cells. Again, the number of any such cells is typically low relative to the number of NK cells present in the population.

As a non-limiting example, at least 60%, at least 70%, at least 80%, at least 85%, at least 90%, at least 91%, at least 92%, at least 93%, at least 94%, at least 95%, at least 96%, at least 97%, at least 98%, at least 99% or more, up to 100% of the cells of an expanded NK cell population of the invention may be NK cells, such as CD16⁺ NK cells. Typically at least 80%, preferably at least 85%, more preferably at least 90%, or even more preferably at least 95% of the cells of an expanded NK cell population of the invention are NK cells, such as CD16⁺ NK cells.

In some embodiments, at least 60%, at least 70%, at least 80%, at least 85%, at least 90%, at least 91%, at least 92%, at least 93%, at least 94%, at least 95%, at least 96%, at least 97%, at least 98%, at least 99% or more, up to 100% of the cells of an expanded NK cell population of the invention are mature NK cells (i.e. mNK cells and/or cNK cells). Preferably at least 80%, more preferably at least 85%, even more preferably at least 90%, even more preferably at least 95%, even more preferably at least 98% or more of the cells of an expanded NK cell population of the invention are mature NK cells, such as mature CD16⁺ NK cells.

The number of HPCs (including HSCs, CLP cells and/or NKPs) may be less than 40%, less than 30%, less than 25%, less than 20%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% of the cells of the expanded NK cell population. Typically the number of HPCs (including HSCs, CLP cells and/or NKPs) is less than 20%, preferably less than 15%, more preferably less than 10%, even more preferably less than 5%, even more preferably less than 2% or less of the cells of the expanded NK cell population.

The number of other immune and/or non-immune cells may be less than 40%, less than 30%, less than 25%, less than 20%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% of the cells of the expanded NK cell population. Typically the number of other immune and/or non-immune cells is less than 20%, preferably less than 15%, more preferably less than 10%, even more preferably less than 5% of the cells, even more preferably less than 2%, or less of the expanded NK cell population.

As described herein, the expanded NK cell populations made by the methods of the present invention offer several advantages over NK cell populations made by conventional adoptive transfer methods. In particular, the methods of the present invention enable the production of expanded populations with greater number of NK cells compared with conventional methods. Further, a greater proportion of the NK cells (e.g. CD16⁺ NK cells) in a population of the invention are functional, preferably fully functional, compared with populations obtained by conventional methods, in which a large number of the NK cells are “exhausted”.

As used herein, the term “exhausted” in the context of NK cells means that an NK cell or expanded NK cell population has lost at least some of its effector functions, such as cytotoxic function, cytokine production and/or ADCC. Thus, an exhausted NK cell or expanded NK cell population may exhibit impaired survival, impaired cytotoxic function, altered or impaired cytokine production and/or impaired ADCC. For example, an exhausted NK cell or exhausted NK cell population may exhibit at least a 50% reduction in one of its effector functions. For example, at least a 50% reduction in cytokine secretion, at least a 50% reduction in ADCC and/or at least 50% reduction in cytotoxic activity. These values may be quantified relative to any appropriate control as defined herein. Any appropriate technique can be used to determine effector function, and hence to quantify and reduction therein. Suitable techniques are known in the art. Alternatively and/or in addition, exhausted NK cells may exhibit altered marker expression, such as an increase in the expression of one or more inhibitory receptor (as described herein) and/or a decrease in the expression of one or more activatory receptor (as described herein). In some embodiments, increased expression of NKG2A and/or Tim3 may be used as a marker for NK cell exhaustion. Again, the expression of these markers may be quantified relative to any appropriate control as defined herein.

In contrast, the terms “functional” and “fully functional” in the context of NK cells means that an NK cell or expanded NK cell population has all of the expected effector functions when responding to a given immune challenge. Thus, a (fully) functional NK cell or expanded NK cell population will typically exhibit cytotoxic function, cytokine production and/or ADCC as would be observed in vivo when NK cells are activated in response to an immune challenge, and will typically exhibit enhanced survival compared with NK cells produced using conventional methods. Alternatively and/or in addition, (fully) functional NK cells may exhibit altered marker expression, such as an increase in the expression of one or more activatory receptor (as described herein) and/or a decrease in the expression of one or more inhibitory receptor (as described herein). As a non-limiting example, a functional (mature) human NK cell may be CD56⁺ and/or CD45⁺, preferably both CD56⁺ and CD45⁺.

Alternatively or in addition to detecting and/or quantifying CD16⁺ to determine the functionality of the NK cells, the cytotoxicity of NK cells can be determined using a degranulation assay in NK cells co-incubated with ‘target cells’. A degranulation assay involves analysing the expression of CD107a within the NK cell population. The amount of CD107a correlates with cytokine secretion and NK cell-mediated lysis of target cells. NK cells can also be analysed for the expression of Interferon-γ (IFN-γ), which is the main cytokine secreted when functional NK cells are activated. NK cells that are functional should express similar or higher CD107a as well as IFN-γ when compared to a control. Further alternatively or additionally, the cytotoxicity of the NK cells may be determined using flow cytometry to quantify cell death in a target cell population. Said target cell population may optionally be pre-labelled with fluorescent marker. In some embodiments, the use of flow cytometry to quantify cell death in a target cell population is a preferred means of determining the cytotoxicity of the NK cells.

Any increase in NK cell (e.g. CD16⁺ NK cell) number/functionality in an expanded NK cell (e.g. CD16⁺NK cell) population made by a method of the present invention may be compared with the NK cell number/function of an NK cell population obtained from a control method as described herein. A control method may be any standard method known in the art for producing NK cell populations. For example, a control method may use conventional adoptive transfer techniques, rather than a method using a REV-ERB inhibitor according to the present invention. NK cells and NK cell populations produced by such control/standard methods may be used as control cells and populations as described herein.

As an expanded NK cell (e.g. CD16⁺ NK cell) population of the present invention comprises significantly fewer exhausted NK cells compared to conventionally prepared NK cell populations, but instead contains a higher proportion of fully functional NK cells, this advantageously allows the use of smaller numbers of cells to treat patients.

As described herein, the methods of the invention produce expanded NK cell populations with a higher proportion of (fully) functional NK cells compared with conventional methods, which produce populations with large numbers of “exhausted” NK cells. Typically, in an expanded NK (e.g. CD16⁺ NK cell) cell population of the invention at least 50%, at least 60%, at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more, up to 100% of the NK cells of an expanded NK cell population of the invention are (fully) functional. Typically at least 80%, preferably at least 85%, more preferably at least 90%, even more preferably at least 95%, even more preferably at least 98% or more of the NK cells of an expanded NK cell population of the invention are fully functional, according to any definition (e.g. marker and/or effector function definition) herein.

The functionality of the NK cells of the invention may correlate with CD16 expression. By way of non-limiting example, at least 40% of the NK cells in a CD16⁺ NK cell population may be functional and at least 40% of the NK cells may be CD16⁺. Alternatively, the proportion of functional NK cells may be greater than the proportion of CD16⁺ NK cells. By way of non-limiting example, at least 60% of the NK cells in a CD16⁺ NK cell population may be functional and at least 40% of the NK cells may be CD16⁺. By way of a further alternative, the proportion of CD16⁺ NK cells may be greater than the proportion of functional NK cells. By way of non-limiting example, at least 40% of the NK cells in a CD16⁺ NK cell population may be functional and at least 60% of the NK cells may be CD16⁺.

An expanded NK cell population of the invention may be produced by any of the methods disclosed herein. Typically an expanded NK cell population of the invention is produced by an ex vivo method as disclosed herein.

### Notch Ligand

The Notch signalling pathway is primarily associated with promoting T cell development and repressing concomitant B cell development. Mammals have four types of Notch receptor—Notch1, Notch2, Notch3 and Notch4, all of which are single-pass heterodimeric transmembrane protein. Mammals have two types of canonical Notch ligands—Delta type and Jagged type, collectively known as DSL ligands. There are three delta-like ligands (DLLs), DLL1, DLL3 and DLL4 and two jagged (JAG) ligands, JAG1 and JAG2. DLL and JAG ligands typically comprise the following domains: a module at the N-terminus of Notch ligand (MNNL) domain and a Delta/Serrate/Lag-2 (DSL) domain, together with a number of EGF repeats. DLL3 comprises six EGF repeats. DLL1 and DLL4 comprise eight EGF repeats. JAG1 and JAG2 comprise 16 EGF repeats. There are also numerous non-canonical ligands, which may be membrane-bound or secreted.

Unless explicitly stated herein, a reference herein to a Notch ligand is a reference to any Notch ligand, such as a ligand of Notch1, Notch2, Notch3 and/or Notch 4, preferably a ligand of at least Notch1. The protein sequence of human Notch1 is given in SEQ ID NO: 51 (GenBank Accession No. CR457221, version CR457221.1). Typically the Notch ligand of use in the present invention is a canonical Notch ligand. In some preferred embodiments, the Notch ligand is a DLL, more preferably DLL4. The protein sequence of human DLL4 is given in SEQ ID NO: 2 (GenBank Accession No. AF253468, version AF253468.1).

A reference herein to a Notch ligand also embraces fragments thereof, provided said fragment retains the Notch-binding and activatory activity of the Notch ligand from which it is derived. As a non-limiting example, a Notch ligand fragment of the invention may comprise or consist of 170 consecutive amino acid residues or more in length (e.g. at least 170, 180, 190, 200, 210, 220, 230, 240, 250, 260, 270, 280,290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600,610, 620, 630, 640,650, 660, 670, 680, or more consecutive amino acid residues in length, up to the total length of the Notch ligand, such as DLL4. Typically a functional fragment of a Notch ligand comprises at least 50%, at least 55%, at least 60%, at least 65%, at least 66%, at least 67%, at least 68%, at least 69%, at least 70% or more of the full-length Notch ligand, such as DLL4. As a non-limiting example, a fragment of a Notch ligand may comprise the MNNL domain and/or the DSL domain of said Notch ligand, such as DLL4. In some preferred embodiments, the Notch ligand fragment comprises (or consists of) both the MNNL and DSL domains of a Notch ligand of interest, such as the MNNL and DSL domains of DLL4. Examples of such fragments include, but are not limited to the following: (i) a Notch ligand fragment comprising or consisting of the portion of a Notch ligand of interest starting at the N-terminus and terminating at the end of the first EGF repeat, otherwise referred to as Notch Ligand (N-EGF1), such as DLL4 (N-EGF1); (ii) a Notch ligand fragment comprising or consisting of the portion of a Notch ligand of interest starting at the N-terminus and terminating at the end of the second EGF repeat, otherwise referred to as Notch Ligand (N-EGF2), such as DLL4 (N-EGF2); (iii) a Notch ligand fragment comprising or consisting of the portion of a Notch ligand of interest starting at the N-terminus and terminating at the end of the third EGF repeat, otherwise referred to as Notch Ligand (N-EGF3), such as DLL4 (N-EGF3); (iv) a Notch ligand fragment comprising or consisting of the portion of a Notch ligand of interest starting at the N-terminus and terminating at the end of the fourth EGF repeat, otherwise referred to as Notch Ligand (N-EGF4), such as DLL4 (N-EGF4); (v) a Notch ligand fragment comprising or consisting of the portion of a Notch ligand of interest starting at the N-terminus and terminating at the end of the fifth EGF repeat, otherwise referred to as Notch Ligand (N-EGF5), such as DLL4 (N-EGF5); (vi) a Notch ligand fragment comprising or consisting of the portion of a Notch ligand of interest starting at the N-terminus and terminating at the end of the sixth EGF repeat, otherwise referred to as Notch Ligand (N-EGF6), such as DLL4 (N-EGF6); or (vii) a Notch ligand fragment comprising or consisting of the portion of a Notch ligand of interest starting at the N-terminus and terminating at the end of the seventh EGF repeat, otherwise referred to as Notch Ligand (N-EGF7), such as DLL4 (N-EGF7). By way of comparison, the full-length DLL4 may be referred to as DLL4 (N-EGF8). Preferred examples of Notch ligand fragments include Notch ligand (N-EGF1) and Notch ligand (N-EGF2), such as DLL4 (N-EGF1) and DLL4 (N-EGF2).

Alternatively or in addition, a Notch ligand, fragment thereof, or molecule that mimics the effect (e.g. function/activity) of a Notch ligand, such as DLL4 may comprise modifications, such as amino acid mutations which alter, typically increase, the affinity of the ligand/fragment/mimetic for its Notch receptor. Techniques for identifying such modifications are known in the art. For example, amino acids which increase the affinity of a Notch ligand/fragment/mimetic can be identified using yeast surface display. As a non-limiting example, in instances where the Notch ligand of interest is DLL4, the DLL4 ligand of the invention, a fragment or mimetic thereof may comprise an amino acid substitution at one or more of the following positions: (G)28, (F)107, (N)118, (I)143, (H)194, (L)206 and/or (K)215, or any combination thereof. In some preferred embodiments, the DLL4 ligand of the invention, a fragment or mimetic thereof comprises amino acid substitutions at positions (G)28, (F)107, and (L)206, more preferably at positions (G)28, (F)107, (N)118, (I)143, (H)194, (L)206 and (K)215. As a further non-limiting example, in instances where the Notch ligand of interest is DLL4, the DLL4 ligand of the invention, a fragment or mimetic thereof may one or more of the following amino acid substitutions G28S, F107L, N118I, I143F, H194Y, L206P and/or K215E, or any combination thereof. In some preferred embodiments, the DLL4 ligand of the invention, a fragment or mimetic thereof comprises the amino acid substitutions, G28S, F107L and L206P, more preferably G28S, F107L, N118I, I143F, H194Y, L206P and/or K215E.

As a further non-limiting example, a functional fragment of DLL4 comprises at least residues 65 to 114 and 179 to 219 of full-length DLL4, preferably held in the correct conformation to allow interaction with the Notch ligand.

In addition, the invention encompasses the use of molecules that would mimic the effect (e.g. activity/function) of a Notch ligand (also referred to herein as mimetics). For example, the use of peptides, stapled peptides, peptoids and peptidomimetics that would mimic the effect of the desired Notch ligand (such as DLL4) is embraced by the present invention. Methods for producing synthetic peptides and peptidomimetics (such as peptoids) are known in the art, as are the sequences of canonical and non-canonical Notch ligands. Thus, it would be routine for one of skill in the art to produce suitable molecules which mimic the effect of a desired Notch ligand using known techniques and based on the known Notch ligand sequences. As a non-limiting example, peptidomimetics may be designed to interact with key residues of Notch (e.g. Notch1) that are known to be involved in binding to DLL4, such as one or more of residues 415 (E415), 418 (L418), 420 (A420), 421 (N421), 422 (P422), 424 (E424), 425 (H425), 436 (F436), 447 (P447), 448 (R448), 450 (E450), 452 (D452), 469 (D469), 477 (I477), 480 (P480) of Notch (Notch1), or any combination thereof.

Peptidomimetics are described herein in relation to REV-ERB inhibitors. That disclosure applies equally and independently to peptidomimetics of Notch ligands.

The methods of the invention may encompass the use of any Notch ligand or fragment thereof which is capable of increasing NK cell production or molecule which mimics the effects thereof, particularly which may act synergistically with a compound of the invention which inhibits REV-ERB activity as disclosed herein, or a compound which results in the post-translational modification of E4bp4, and hence an increase in E4bp4 activity as disclosed herein.

The present inventors have shown that E4bp4 directly binds to the regulatory region of the Notch1 gene in vivo and so could enhance the transcriptional regulation of Notch, and that Notch1 expression E4bp4^{-/-} mice is significantly reduced. Following on from this, the present inventors found that short-term exposure of Notch ligands to murine HSCs and very early progenitors can promote NK cell development, even in the absence of the critical transcription factor E4bp4. Further, the present inventors have shown that the Notch ligand Delta-like ligand 4 (DLL4) is particularly effective in stimulating the expansion of NK cells.

Accordingly, the present invention relates to the expansion of NK cells by exposure of the HPCs to a Notch ligand. In ex vivo or in vitro methods of the invention, this can comprise culturing the HPCs in the presence of a Notch ligand. For in vivo methods, this may comprise administering the compound together with a Notch ligand. In preferred embodiments, the Notch ligand is DLL4, or a fragment or variant thereof which retains the function of DLL4.

The present invention also relates to increasing the number of CD16⁺ NK cells in an expanded NK population, or the expansion of CD16⁺ NK cells, by exposure of the HPCs to a Notch ligand in a method which comprises a step of culturing HPCs in a medium that does not induce the differentiation of the HPCs, particularly which does not induce the differentiation of the HPCs to NK cells. Said method may comprise exposing the HPCs to a Notch ligand as part of the step of culturing HPCs in a medium that does not induce the differentiation of the HPCs, particularly which does not induce the differentiation of the HPCs to NK cells. Said methods may further comprise the use of a compound which inhibits the action of REV-ERB as described herein. In said methods, the compound which inhibits the action of REV-ERB may be used before the HPCs are differentiated to produce NK cells or during the differentiation of the HPCs to NK cells. Preferably, the compound which inhibits the action of REV-ERB may be used before the HPCs are differentiated to produce NK cells

In some embodiments, the methods of the invention comprise exposing the HPCs to DLL4, or a fragment or variant thereof which retains the function of DLL4. For example, in the ex vivo or in vitro methods of the invention, the HPCs may be cultured in the presence DLL4, or a fragment or variant thereof which retains the function of DLL4. For the ex vivo and in vitro methods of the invention, an increase in NK cell expansion can be achieved by coated the vessel in which the HPCs are cultured (i.e. the culture vessel) with the Notch ligand, such as DLL4 or a functional fragment or variant thereof.

Variant sequences are described herein in relation to REV-ERB inhibitors. That disclosure applies, inter alia, equally and independently to variants of Notch ligands and fragments/mimetics thereof. The variant Notch ligands/fragments/mimetics of the invention typically at least retain the activity of the corresponding Notch ligands/fragments/mimetics of the invention. Thus, for example, the variant DLL4 ligands or fragments thereof of the invention retain the ability of the corresponding DLL4 molecules to bind to Notch1, and/or to enhance NK cell production. Thus, the variant DLL4 ligands/fragments/mimetics may retain at least 50%, at least 60%, at least 70%, at least 80%, at least 90%, up to and including 100% of the activity of the unmodified DLL4 ligands/fragments/mimetics of the invention. In some embodiments, the variant DLL4 ligands/fragments/mimetics have greater activity than the corresponding unmodified DLL4 ligand/fragment/mimetic. For example, at least 110%, at least 120%, at least 130%, at least 140%, at least 150%, at least 175%, at least 200%, at least 250%, at least 300%, at least 400%, at least 500%, at least 600%, at least 700%, at least 800%, at least 900% or more activity compared with the corresponding unmodified DLL4 ligand/fragment/mimetic. For example, the variant DLL4 ligands/fragments/mimetics may have a K_{D} value for binding to Notch1 that is at least 10-fold lower, at least 15-fold lower, at least 20-fold lower, at least 25-fold lower, at least 30-fold lower, or less than the corresponding unmodified DLL4 ligand/fragment/mimetic. E.g., the variant DLL4 ligands/fragments/mimetics may have a K_{D} value for binding to Notch1 of less than 1 μM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM or less, preferably less than 500 nM, less than 400 nM, less than 300 nM or less. In some embodiments, the variant DLL4 ligands/fragments/mimetics can increase the number of NK cells, or give rise to an increase in NK cell production, of at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 3 fold or more relative to the corresponding unmodified DLL4 ligand/fragment/mimetic. The variant DLL4 ligands/fragments/mimetics may increase number of NK cells by at least 10%, at least 20%, at least 30%, at least 40%, at least 50%, at least 60, at least 70%, at least 80%, at least 85%, at least 90%, at least 95%, at least 100%, at least 150%, at least 200%, at least 300% or more compared with the corresponding unmodified DLL4 ligand/fragment/mimetic. This applies equally to any variants of the other Notch ligands/fragments/mimetics of the invention.

The Notch ligands/fragments/mimetics of the invention may be labelled (or tagged). Any appropriate label may be used. Suitable labels are known in the art.

### E4bp4

E4bp4 (also known as Nfil3) is a basic leucine zipper protein transcription factor which is involved in the regulation of IL-3 expression, and is involved in the coordinating the circadian clock. The genomic DNA sequence of the human E4bp4 gene is given in SEQ ID NO: 3 (Genbank Accession No. X64318, version X64318.1). As shown in FIG. 1, E4bp4 is expressed in CLPs and is critical in the production of NK cells from blood stem cell progenitors. Mice with the E4bp4 gene deleted do not have functional NK cells, but have normal numbers of T and B cells. In contrast, overexpression of E4bp4 in HSCs in vitro increases NK cell production. Thus, E4bp4 is a lineage commitment factor, controlling the development of NKPs from HSCs (FIG. 1). E4bp4's critical function in NK cells is specific to the early stages of the developmental pathway, as specific ablation of E4bp4 in peripheral mNK cells does not affect NK cell number or response to cytomegalovirus infection. In addition E4bp4 regulates other transcription factors that are essential in NK cell development, such as Id2 and Eomes.

Although IL-7 and IL-15 have been shown to regulate E4bp4 expression, generally very little is known about how either extrinsic or intrinsic stimuli influence E4bp4. Transcription factors such as E4bp4 can be hard to target because of their structure and function. For example, they usually lack enzymatic activity or cofactor binding sites. The present inventors have previously demonstrated that E4bp4 expression can be increased using a compound which inhibits the activity of REV-ERB, and that this results in an increase in NK cell number (see GB Application No. 1703476.0, which is herein incorporated by reference in its entirety). Without wishing to be bound by theory, REV-ERB binds to porphyrin heme, and it is this characteristic that is believed to make REV-ERB a druggable target (see below). In sum, the inventors have shown that by targeting REV-ERB and inhibiting its activity, it is possible to increase E4bp4 expression and hence increase NK cell number. Accordingly, the present invention is concerned with compounds which inhibit the action of REV-ERB, and their use in increasing E4bp4 expression, and hence NK cell number.

### Increase in E4bp4 Expression

Accordingly, the present invention provides ex vivo methods for producing expanded NK cell (e.g. CD16⁺ NK cell) populations, and therapeutic methods and applications for increasing NK cell number in a patient in need thereof. As disclosed herein, said methods and applications may involve the use of a compound which inhibits the action of REV-ERB. Typically said compounds act by increasing E4bp4 expression.

An increase in E4bp4 expression may be measured relative to a control. Thus, the expression of E4bp4 in a sample of HPCs, an expanded NK cell population or in a sample obtained from an individual/patient to be treated according to the invention may be compared with the expression of E4bp4 in a control. Expression may be quantified in terms of gene and/or protein expression, and may be compared with expression of a control (e.g. housekeeping gene or protein). The actual amount of the E4bp4 gene, mRNA transcript and/or protein, such as the mass, molar amount, concentration or molarity of the E4bp4 gene, mRNA transcript and/or protein, or the number of mRNA molecules per cell in a sample of HPCs, an expanded NK cell population or in a sample obtained from an individual/patient to be treated according to the invention and the control may be assessed and compared with the corresponding value from the control. Alternatively, the expression of the E4bp4 gene and/or protein in a sample of HPCs, an expanded NK cell population or in a sample obtained from an individual/patient to be treated according to the invention may be compared with that of the control without quantifying the mass, molar amount, concentration or molarity of the one or more gene and/or protein.

Typically the control is an equivalent population or sample in which no increase in E4bp4 expression has been effected. As a non-limiting example, in the case where an individual/patient is treated with a compound that inhibits REV-ERB activity in order to increase E4bp4 expression, a suitable control would be a different individual to which the compound has not been administered or the same individual prior to administration of the compound. Conventional methods for the ex vivo expansion of NK cells, including known methods may be considered control methods according to the present invention.

In the context of the present invention, a reference to increasing E4bp4 expression may be understood to mean that, the expression of E4bp4 is increased by at least 10%, at least 20%, at least 30%, at least 40%, at least 50%, at least 60, at least 70%, at least 80%, at least 85%, at least 90%, at least 95%, at least 100%, at least 150%, at least 200% compared with the control. Typically E4bp4 expression is increased by at least 50%, preferably at least 70%, more preferably at least 80%, even more preferably at least 90% or more compared with the control.

A reference to increasing E4bp4 expression may be understood to mean that, the expression of E4bp4 is increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically E4bp4 gene expression is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control. Typically E4bp4 protein expression is increased by at least 2-fold, at least 3-fold, preferably at least 5-fold, more preferably at least 6-fold or more compared with the control.

The expression of the E4bp4 gene and/or protein according to the invention may be determined by quantitative and/or qualitative analysis. Typically, gene expression may be expressed in terms of mRNA levels.

The expression level of the E4bp4 gene and/or protein according to the invention encompasses the mass of the E4bp4 mRNA transcript and/or protein, the molar amount of the E4bp4 gene, mRNA transcript and/or protein, the concentration of the E4bp4 gene and/or protein and the molarity of the E4bp4 gene and/or protein. This expression level may be given in any appropriate units. For example, the concentration of the E4bp4 gene and/or protein may be given in μg/ml, ng/ml or μg/ml.

The expression level of the E4bp4 gene and/or protein according to the invention may be measured directly or indirectly.

The relative expression of the E4bp4 gene and/or protein according to the invention relative to a control may be determined using any appropriate technique. Suitable standard techniques are known in the art, for example Western blotting, enzyme-linked immunosorbent assays (ELISAs) and RT-qPCR.

The expression level of the E4bp4 gene and/or protein may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the expression level of the E4bp4 gene and/or protein is increased for at least 12 to 72 hours. Typically this is assessed relative to the last administration of the compound which inhibits REV-ERB activity.

The expression level of the E4bp4 gene and/or protein may be increased compared with a control for at least one, at least two, at least three, at least four, at least five, at least ten, at least 20, at least 30, at least 40 or more passages of the NK cell precursors in culture. The expression level of the E4bp4 gene and/or protein may be altered indefinitely.

### REV-ERB

REV-ERB proteins are members of the nuclear receptor family of intracellular transcription factors. The mRNA sequence of the human REV-ERBα gene (Nr1d1) is given in SEQ ID NO: 5 (Genbank Accession No. NM_021724, version NM_021724.4). The mRNA sequence of the human REV-ERBβ gene (Nr1d2) is given in SEQ ID NO: 7 (Genbank Accession No. AB307693, version AB307693.1). REV-ERB regulates the circadian clock, and has also been implicated in the regulation of cartilage breakdown.

The present inventors have previously demonstrated that inhibition of REV-ERB activity is sufficient to elicit a significant increase in E4bp4 expression, and that this in turn brings about an expansion of NK cells, resulting in an increase in NK cell number (see GB Application No. 1703476.0 and WO2018/158587, which are herein incorporated by reference in their entirety). Inhibition of REV-ERB activity can bring about an increase in NK cell number, and that typically the resulting NK cells are (fully) functional as defined herein. The effect of REV-ERB inhibition is mediated in an E4pb4-dependent manner. Without wishing to be bound by theory, it is believed that inhibition of REV-ERB activity results in an increase in E4bp4 expression (E4bp4 expression is normally repressed by REV-ERB), and that the E4bp4 acts to stimulate the production of NK cells (as shown in FIG. 1). In particular, the present inventors have previously demonstrated that the small molecule SR8278 is capable of binding to the porphyrin heme moiety of REV-ERB, resulting in inhibition of REV-ERB activity and an increase in NK cell number

Accordingly, in some embodiments, the present invention is concerned with compounds which inhibit the action of REV-ERB, and their use in increasing E4bp4 expression, and hence NK cell number.

### Inhibition of REV-ERB Activity

In some embodiments, the present invention relates to the use of compounds to inhibit the action of REV-ERB, i.e. compounds which inhibit REV-ERB activity. REV-REB activity may be inhibited by any appropriate means. Suitable standard techniques are known in the art. Inhibition may take place via any suitable mechanism, depending for example on the nature (see below) of the compound used, e.g. steric interference in any direct or indirect interaction or inhibition of REV-ERB. In the context of the present invention a REV-ERB inhibitor (interchangeably referred to herein as a REV-ERB antagonist) is any compound which inhibits, decreases, suppresses or ablates the action of REV-ERB, whether in part or completely.

A decrease in REV-ERB activity may be measured relative to a control. Thus, the activity of REV-ERB in a sample of NK precursor or progenitor cells, an expanded NK cell population or in a sample obtained from an individual/patient to be treated according to the invention may be compared with the activity of REV-ERB in a control. Activity may be quantified in any appropriate terms, for example binding of REV-ERB to the E4bp4 gene, or in terms of E4bp4 expression as defined herein. Any appropriate technique or method may be used for quantifying REV-ERB activity. Suitable techniques are known in the art, for example luciferase assays for quantifying expression of a reporter gene.

Typically the control is an equivalent population or sample in which no REV-ERB inhibitory compound has been added, for example a sample obtained from a different individual to which the compound has not been administered, or the same individual the prior to administration of the compound. Conventional methods for the ex vivo expansion of NK cells, including known methods may be considered control methods according to the present invention.

In the context of the present invention, a reference to inhibiting REV-ERB activity may be understood to mean that, the activity of REV-ERB is decreased by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%) inhibition of REV-ERB activity, as compared with the control. Typically REV-ERB activity is decreased by at least 50%, preferably at least 70%, more preferably at least 80%, more preferably at least 90%, even more preferably at least 95% or more compared with the control.

The activity of REV-ERB may be determined by quantitative and/or qualitative analysis, and may be measured directly or indirectly.

The activity of REV-ERB relative to a control may be determined using any appropriate technique. Suitable standard techniques are known in the art, such as by quantifying E4bp4 expression, and/or luciferase assays.

The activity of REV-ERB may be inhibited compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the activity of REV-ERB is decreased for at least 12 to 72 hours. Typically this is assessed relative to the last administration of the compound which inhibits REV-ERB activity.

The activity of REV-ERB may be inhibited compared with a control for at least one, at least two, at least three, at least four, at least five, at least ten, at least 20, at least 30, at least 40 or more passages of the cells (either in vivo, or cultured ex vivo or in vitro). The activity of REV-ERB may be inhibited and/or the expression level of the E4bp4 gene and/or protein may be altered indefinitely.

In the context of the present invention any reference to inhibiting REV-ERB activity may be understood to mean inhibiting the activity of REV-ERBα and/or REV-ERBO. In preferred embodiments, the activity of both REV-ERBα and REV-ERBO is inhibited. Thus, the invention relates to compounds which inhibit REV-ERB activity, including compounds which inhibit REV-ERBα activity (i.e. REV-ERBα inhibitors, also referred to as REV-ERBα antagonists) and/or to compounds which inhibit REV-ERBO activity (i.e. REV-ERBO inhibitors, also referred to as REV-ERBβ antagonists). In preferred embodiments, the invention relates to compounds which inhibit the activity of both REV-ERBα and REV-ERBO (i.e. REV-ERBα and REV-ERBO inhibitors, also referred to as REV-ERBα and REV-ERBβ antagonists).

### REV-ERB Antagonists/Inhibitors

REV-ERB inhibitory compounds of the invention may be specific for REV-ERB. By specific, it will be understood that the compound binds to REV-ERBα and/or REV-ERBβ, with no significant cross-reactivity to any other molecule, particularly any other protein. For example, modulator that is specific for REV-ERBα and/or REV-ERBβ will show no significant cross-reactivity with human neutrophil elastase. Cross-reactivity may be assessed by any suitable method. Cross-reactivity of REV-ERBα and/or REV-ERBO inhibitor with a molecule other than REV-ERBα and/or REV-ERBO may be considered significant if the inhibitor binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to REV-ERBα and/or REV-ERBO. An inhibitor that is specific for REV-ERBα and/or REV-ERBβ may bind to another molecule such as human neutrophil elastase at less than 90%, 85%, 80%, 75%, 70%, 65%, 60%, 55%, 50%, 45%, 40%, 35%, 30%, 25% or 20% the strength that it binds to REV-ERBα and/or REV-ERBO. Preferably, the inhibitor binds to the other molecule at less than 20%, less than 15%, less than 10% or less than 5%, less than 2% or less than 1% the strength that it binds to REV-ERBα and/or REV-ERBO.

REV-ERB inhibitory compounds of the invention may have off-target effects. An off-target effect is activity against a target other than REV-ERB. Typically compounds with off-target effects are encompassed by the present invention if the activity against the non-REV-ERB target is not significant compared with the activity against REV-ERB. Whether an off-target effect is significant may depend on the intended use of the compound. As a non-limiting example, a compound which may exert an off-target effect on the central nervous system would not be significant for a compound used in an ex vivo method as disclosed herein, but may be significant (depending on the magnitude of the off-target effect) for an in vivo therapeutic indication as disclosed herein. The presence and magnitude of any potential off target effects can be readily assessed using standard methods known in the art.

Any suitable inhibitor may be used according to the invention, for example small molecules, PROTAC reagents, double stranded RNA (dsRNA), small interfering RNA (siRNA), small hairpin RNA (shRNA), microRNA, antisense (single stranded) RNA, peptides and peptidomimetics, antibodies, aptamers and ribozymes. Preferred inhibitors include small molecules and PROTAC reagents.

### Small Molecules

Small molecules may be used to in it REV-ERB activity as described herein. As defined herein, small molecules are low molecular weight compounds, typically organic compounds. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

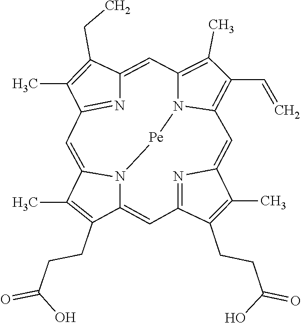

According to the present invention, small molecules may be able to exert an inhibitory effect on REV-ERB activity by binding to the porphyrin heme moiety of REV-ERB. Thus in some preferred embodiments, a compound that inhibits the action of REV-ERB according to the present invention is a compound which binds to the porphyrin heme moiety of REV-ERB, and hence inhibits the activity of REV-ERB. Alternatively, the small molecule may act via a different mechanism, for example, by binding to a non-heme portion of REV-ERB. Standard techniques are known in the art for the production of small molecules, which can then readily be tested for REV-ERB inhibitory activity as described herein

### Structure of Porphyrin Heme

In a preferred embodiment, the invention relates to the small molecule 1,2,3,4-Tetrahydro-2-[[5-(methylthio)-2-thienyl]carbonyl]-3-isoquinolinecarboxylic acid ethyl ester, herein referred to as SR8278 as a REV-ERB inhibitor.

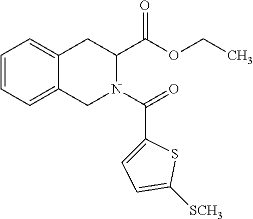 [SMILES: CCOC(=O)C1Cc2ccccc2CN1C(=O)c1ccc(SC)s1]

### Structure of SR8278

The invention also encompasses the use of variants of SR8278 which retain the REV-ERB inhibitory function of SR8278.

Preferably, the invention encompasses the use of improved REV-ERB antagonists as described in WO2020/002911, particularly the Examples, which is herein incorporated by reference in its entirety. In particular, the use of compounds 11 and 7 of WO2020/002911 is encompassed by the present invention. Compounds 11 and 7 of WO2020/002911 have the following structures:

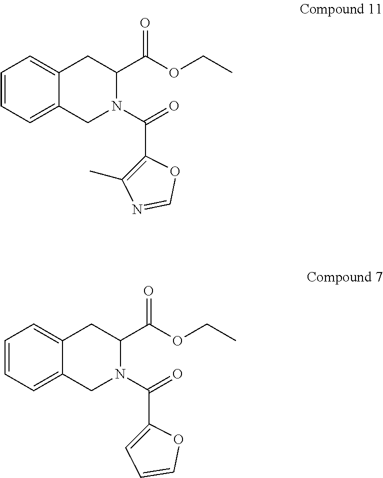 [SMILES: CCOC(=O)C1Cc2ccccc2CN1C(=O)c1ocnc1C ; CCOC(=O)C1Cc2ccccc2CN1C(=O)c1ccco1]

Any small molecule which exerts an inhibitory effect on REV-ERB activity may be used as a REV-ERB inhibitor according to the present invention. Such small molecule inhibitors may also bind to REV-ERB. Examples of other small molecules which may be used as REV-ERB inhibitors according to the present invention include 4-[[[1-(2-fluorophenyl)cyclopentyl]amino]methyl]-2-[(4-methylpiperazin-1-yl)methyl]phenol (also referred to herein as ARN5187), ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine and 1-(4-fluorophenyl)-N-(3-((1-methylpiperidin-4-yl)methyl)benzyl)cyclopentan-1-amine.

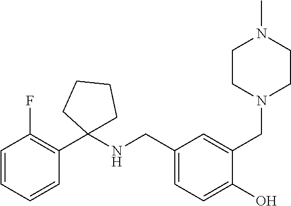 [SMILES: CN1CCN(Cc2cc(CNC3(c4ccccc4F)CCCC3)ccc2O)CC1]

### Structure of ARN5187

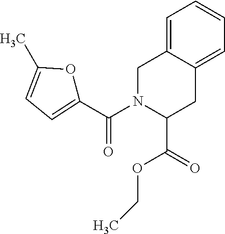 [SMILES: CCOC(=O)C1Cc2ccccc2CN1C(=O)c1ccc(C)o1]

### Structure of ethyl 2-(5-methylfuran-2-carbonyl)-1,2,3,4-tetrahydroisoquinoline-3-carboxylate

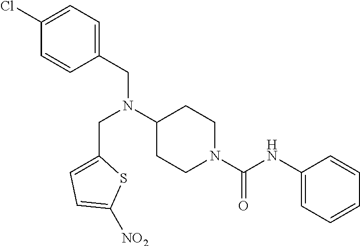 [SMILES: O=C(Nc1ccccc1)N1CCC(N(Cc2ccc(Cl)cc2)Cc2ccc([N+](=O)[O-])s2)CC1]

### Structure of 4-((4-chlorobenzyl)((5-nitrothiophen-2-yl)methyl)amino)-N-phenylpiperidine-1-carboxamide

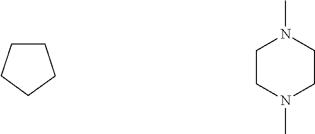

### Structure of 4-(((1-(4-fluorophenyl)cyclopentyl)amino)methyl)-2-((4-methylpiperazin-1-yl)methyl)phenol

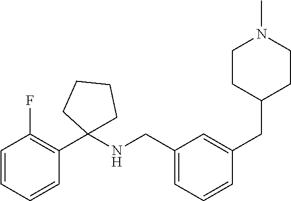 [SMILES: CN1CCC(Cc2cccc(CNC3(c4ccccc4F)CCCC3)c2)CC1]

### Structure of 1-(2-fluorophenyl)-N-(3-((1-methylpiperidin-4-yl)methyl)benzyl)cyclopentan-1-amine

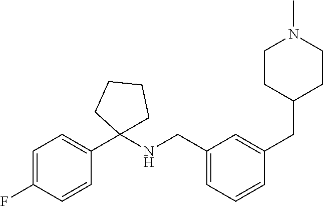 [SMILES: CN1CCC(Cc2cccc(CNC3(c4ccc(F)cc4)CCCC3)c2)CC1]

### Structure of 1-(4-fluorophenyl)-N-(3-((1-methylpiperidin-4-yl)methyl)benzyl)cyclopentan-1-amine

The invention also encompasses the use of variants of ARN5187, ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine or 1-(4-fluorophenyl)-N-(3-((1-methylpiperidin-4-yl)methyl)benzyl)cyclopentan-1-amine which retain the REV-ERB inhibitory function of ARN5187, ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine or 1-(4-fluorophenyl)-N-(3-((1-methylpiperidin-4-yl)methyl)benzyl)cyclopentan-1-amine respectively.

### PROTAC Reagents

Proteolysis targeting chimeras (also referred to as PROTACs or PROTAC reagents) may be used to inhibit REV-ERB activity as described herein. PROTACs are heterobifunctional small molecules that simultaneously bind a target protein and ubiquitin ligase, enabling ubiquitination and degradation of the target. In more detail, a PROTAC reagent typically comprises a ligand for the target protein (in the case of the present invention, REV-ERB) and a ligand for an E3 ligase recognition domain. Through the use of such a PROTAC, an E3 ligase is recruited to the PROTAC-bound REV-ERB, inducing ubiquitin transfer from the E3 ligase complex to the target protein (in the case of the present invention, REV-ERB). Once the PROTAC has induced a sufficient degree of ubiquitination of the target, it is then recognised and degraded by the proteasome.

As a non-limiting example, a PROTAC reagent may be produced by conjugating a ligand for an E3-ligase to a small molecule inhibitor as described herein (preferably SR8278) via a linker. In a preferred embodiment, a PROTAC reagent comprises a ligand for the E3 RING Cullin ligase von-Hippel Lindau protein (VHL) or cereblon—a part of a CRL4 E3 RING Cullin ligase complex, connected to a small molecule inhibitor of the invention via a linker. In some particularly preferred embodiments, the PROTAC reagent comprises a ligand for the E3 RING Cullin ligase von-Hippel Lindau protein (VHL) connected to SR8278, connected via a linker. In other particularly preferred embodiments, the PROTAC reagent comprises cereblon (a part of a CRL4 E3 RING Cullin ligase complex) and SR8278, connected via a linker.

Because of their mechanism of action, PROTAC reagents simply need any ligand for the target protein. The functional pharmacology of the ligand, in the absence of the linker and E3 ligase ligand, is unimportant. Therefore in some embodiments a REV-ERB inhibitory PROTAC reagent of the present invention may comprises a small molecule REV-ERB agonist as the ligand, such as GSK4112 (1,1-Dimethylethyl N-[(4-chlorophenyl)methyl]-N-[(5-nitro-2-thienyl)methyl])glycinate, SR6452).

### Double-Stranded RNA

Double-stranded RNA (dsRNA) molecules may be used to inhibit REV-ERB activity as described herein. dsRNA molecules may be used in RNAi to inhibit REV-ERB activity.

Using known techniques and based on a knowledge of the sequence of REV-ERB, dsRNA molecules can be designed to antagonise REV-ERB by sequence homology-based targeting of the corresponding RNA sequence. Such dsRNAs will typically be small interfering RNAs (siRNAs), small hairpin RNAs (shRNAs), or micro-RNAs (miRNAs). The sequence of such dsRNAs will comprise a portion that corresponds with that of a portion of the mRNA encoding REV-ERB. This portion will usually be 100% complementary to the target portion within the mRNA transcribed from the REV-ERB gene, but lower levels of complementarity (e.g. 90% or more or 95% or more) may also be used. Typically the % complementarity is determined over a length of contiguous nucleic acid residues. A dsRNA molecule of the invention may, for example, have at least 80% complementarity to the target portion within the mRNA transcribed from the REV-ERB gene measured over at least 10, at least 20, at least 30, at least 40, at least 50, at least 60, at least 70, at least 80, at least 90, or more nucleic acid residues, up to the dsRNA molecule having at least 80% complementarity the mRNA transcribed from the REV-ERB gene of the invention over the entire length of the dsRNA molecule.

In a preferred embodiment, the dsRNA is a shRNA. ShRNA can be delivered to NK cell precursors by any appropriate means. Suitable techniques are known in the art and include the use of plasmid, viral and bacterial vectors to deliver the shRNA. Typically, the shRNA is delivered using a viral vector delivery system. In a preferred embodiment, the viral vector is a lentiviral vector.

Generally, once the shRNA has been delivered to an NK precursor cell, it is then transcribed in the nucleus and processed. The resulting pre-shRNA is exported from the nucleus and then processed by dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing.

A variant sequence may have at least 80% sequence identity to an shRNA sequence of the invention, measured over any appropriate length of sequence. Typically the % sequence identity is determined over a length of contiguous nucleic acid or amino acid residues. A variant sequence of the invention may, for example, have at least 80% sequence identity to a sequence of the invention measured over at least 10, at least 20, at least 30, at least 40, at least 50, at least 60, at least 70, at least 80, at least 90, or more nucleic acid or amino acid residues.

For example, a variant shRNA molecule of the invention may have at least 80% sequence identity with an shRNA molecule of the invention measured over at least 10, at least 20, at least 30, at least 40, at least 50, at least 60 or more nucleic acid residues, up to the variant shRNA molecule having at least 80% sequence identity with the shRNA molecule of the invention over the entire length of the variant shRNA molecule.

### Antisense RNA

Single-stranded DNA (ssDNA) molecules, also known as antisense RNA, may be used to inhibit REV-ERB activity as described herein.

Using known techniques and based on a knowledge of the sequence of the REV-ERB gene, antisense RNA molecules can be designed to antagonise the REV-ERB gene by sequence homology-based targeting of the corresponding RNA. The sequence of such antisense will comprise a portion that corresponds with that of a portion of the mRNA transcribed from the REV-ERB gene. This portion will usually be 100% complementary to the target portion within the transcribed mRNA but lower levels of complementarity (e.g. 90% or more or 95% or more) may also be used.

### Aptamers

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. Nos. 5,654,151, 5,503,978, 5,567,588 and WO 96/38579.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Peptidomimetics

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

### Antibodies

Antibodies may be used to inhibit REV-ERB activity as described herein.

As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd or dAb.

### Variant Sequences

A sequence identity of at least 80% includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every sequence presented herein and/or to each and every SEQ ID NO presented herein).

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M—A New Algorithm for Multiple Alignment of Highly Divergent Sequences, 20 (9) Bioinformatics: 1428-1435 (2004). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992.

Variants of the specific sequences provided above may alternatively be defined by reciting the number of nucleotides or amino acids that differ between the variant sequences and the specific reference sequences provided above. Thus, in one embodiment, the sequence may comprise (or consist of) a nucleotide sequence that differs from the specific sequences provided above at no more than 5, no more than 4, no more than 3, no more than 2 nucleotide positions, for example at no more than 1 nucleotide position. Conservative substitutions are preferred.

The variant nucleic acid molecules and peptides of the invention typically still retain the activity of the corresponding molecules of the invention. Thus, for example, the variant shRNA molecules of the invention retain the ability of the corresponding shRNA molecules to inhibit the expression of REV-ERB. The variant shRNA molecules may retain at least 50%, at least 60%, at least 70%, at least 80%, at least 90%, up to and including 100% of the modulatory activity of the shRNA molecules of the invention. This applies equally to any other variants of the inhibitors of the invention.

The compounds of the invention may be labelled (or tagged). Any appropriate label may be used. Suitable labels are known in the art.

### Post-Translational Modification of E4bp4

As shown in the Examples, the present inventors have demonstrated that post-translational modification of E4bp4 can increase E4bp4 activity. Furthermore, increasing E4bp4 activity by post-translational modification results in an increase in NK cell number (as defined herein).

Accordingly, the present invention provides a method of expanding an NK cell population, comprising the steps of: a) culturing an haematopoietic progenitor cell (HPC) comprising sample obtained from an individual/patient; b) contacting said sample with a compound which results in the post-translational modification of E4bp4, thereby causing an increase in E4bp4 activity; and c) expanding said cells in vitro to produce an NK cell population. Said method can be used together with, or independently from the methods disclosed herein relating to increased E4bp4 expression by decreasing REV-ERB activity, and/or the methods disclosed herein relating to increasing NK cell number by culturing HPCs in the presence of a Notch ligand.

The invention also provides a compound which results in the post-translational modification of E4bp4 for use in a method of therapy by increasing production of NK cells in a patient, wherein said compound increases E4bp4 activity. The invention further provides a method of treatment by increasing the number of NK cells in a patient in need thereof, comprising administering to said patient a therapeutically effective amount of a compound which results in the post-translational modification of E4bp4, wherein said compound increases E4bp4 activity. Again, said therapeutic indications can be used together with, or independently from the indications disclosed herein relating to increased E4bp4 expression by decreasing REV-ERB activity, and/or the indications disclosed herein relating to increasing NK cell number by culturing HPCs in the presence of a Notch ligand.

Similarly, a compound which alters or affects the post-translational modification of E4bp4 may therefore be used according to the invention for increasing production of NK cells in a patient, wherein said compound increases E4bp4 activity, or for use in a method of treatment by increasing the number of NK cells in a patient in need thereof, together with the methods disclosed herein relating increasing CD16⁺ NK cell numbers by inclusion of a pre-differentiation step (A), the method disclosed herein relating to increasing CD16⁺ NK cell number comprising a step of increasing E4bp4 expression by decreasing REV-ERB activity and/or the methods disclosed herein relating to increasing CD16⁺ NK cell number by culturing HPCs in the presence of a Notch ligand.

Any of the disclosure herein in relation to methods of increasing NK cell number, methods of expanding NK cells in the context of compounds which inhibit the action of REV-ERB, and/or Notch ligands, expanded NK cell populations produced by said methods and therapeutic indications relating to said compounds and populations applies inter alia to the disclosed methods of increasing E4bp4 activity to increase NK cell number. As non-limiting examples, the feeder cell layers, growth factors and/or other culture conditions and diseases to be treated may be the same in relation to the post-translational modification aspects as for the REV-ERB inhibition and/or Notch ligand aspects disclosed herein.

### Types of Post-Translational Modification

Said method encompasses any post-translational modification which results in an increase in E4bp4 activity. Non-limiting examples of post-translation modification include phosphorylation, SUMOylation, the addition of a hydrophobic group (e.g. myristoylation, palmitoylation), addition of a cofactor, the addition of small chemical groups (e.g. acylation, alkylation, amidation, glycosylation), glycation, carbamylation, carbonylation, chemical modifications (e.g. deamidation) and/or structural changes. Typically said post-translational modification results in a reduction in phosphorylation at one or more phosphorylation site within wild-type (unmodified) E4bp4 and/or a reduction in SUMOylation at one or more SUMOylation site within wild-type (unmodified) E4bp4, or a combination thereof. As shown in the Examples herein, wild-type (unmodified) E4bp4 is typically SUMOylated at one or more of residues K10, K116, K219, K337 and/or K394 or residues corresponding thereto, or any combination thereof. Typically wild-type (unmodified) E4bp4 is SUMOylated at least at residue K219 (or a corresponding residue). Alternatively or in addition, wild-type (unmodified) E4bp4 is typically phosphorylated at residues S286, S301 and S353, or residues corresponding thereto, or any combination thereof. Accordingly, in some preferred embodiments, a compound of the invention reduces, inhibits or ablates SUMOylation at residue K219 (or a residue corresponding thereto), and/or reduces, inhibits or ablates phosphorylation at residues S286, S301 and S353 (or corresponding residues), or any combination thereof. Thus, according to the present invention, a compound may be used to (a) reduce SUMOylation at one or more of residues K10, K116, K219, K337 and/or K394 of E4bp4, or a residue corresponding thereto, or any combination thereof; and/or reduce phosphorylation at one or more of residues S286, S301 and/or S454, or a residue corresponding thereto, or any combination thereof.

Any compound which is capable of eliciting a post-translational modification of E4bp4, wherein said modification increases the activity of E4bp4 is encompassed by the present invention. In some preferred embodiments, said compound inhibits, reduces or ablates the phosphorylation and/or SUMOylation that occurs in wild-type (unmodified) E4bp4. Any appropriate kinase inhibitor may be used to inhibit, reduce or ablate phosphorylation of E4bp4. Suitable kinase inhibitors are known in the art and their selection would be routine to one of skill in the art. Non-limiting examples of suitable kinase inhibitors include 4-(4-(2,3-Dihydrobenzo[1,4]dioxin-6-yl)-5-pyridin-2-yl-1H-imidazol-2-yl)benzamide (D4476) and 4,5,6,7-Tetrabromo-2-azabenzimidazole, 4,5,6,7-Tetrabromobenzotriazole (TBB).

### Increase in E4bp4 Activity

The present invention relates to the use of compounds post-translationally modify E4bp4 and hence to increase E4bp4 activity. An increase in E4bp4 activity may be measured relative to a control. Thus, the activity of E4bp4 in a sample of NK precursor or progenitor cells, an expanded NK cell population or in a sample obtained from an individual/patient to be treated according to the invention may be compared with the activity of E4bp4 in a control. Activity may be quantified in any appropriate terms, for example an increase in the expression of any downstream target of E4bp4. Any appropriate technique or method may be used for quantifying E4bp4 activity. Suitable techniques are known in the art, for example luciferase assays for quantifying expression of a reporter gene.

Typically the control is an equivalent population or sample in which no compound has been added to post-translationally modify E4bp4, for example a sample obtained from a different individual to which the compound has not been administered, or the same individual the prior to administration of the compound. Conventional methods for the ex vivo expansion of NK cells, including known methods may be considered control methods according to the present invention.

In the context of the present invention, a reference to increasing E4bp4 activity may be understood to mean that, the activity of E4bp4 is increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically E4bp4 activity is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control. E4bp4 activity may be measured indirectly be determining the increase in NK cell number. Thus, the number of NK cells may be increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control.

The activity of E4bp4 may be determined by quantitative and/or qualitative analysis, and may be measured directly or indirectly. The activity of E4bp4 relative to a control may be determined using any appropriate technique. Suitable standard techniques are known in the art.

The activity of E4bp4 may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the activity of E4bp4 is increased for at least 12 to 72 hours. Typically this is assessed relative to the last administration of the compound which post-translationally modified E4bp4.

The activity of E4bp4 may be increased compared with a control for at least one, at least two, at least three, at least four, at least five, at least ten, at least 20, at least 30, at least 40 or more passages of the cultured cells. The activity of E4bp4 may be increased indefinitely.

### Methods of Expanding NK Cells

The present invention relates to methods for expanding an NK cell population. Typically said methods involve culturing NK cell precursors (HPCs) in the presence of a Notch ligand. Said method may be in vitro, in vivo or ex vivo. Typically the method of the invention is ex vivo. Said method comprises containing HPCs with a Notch ligand and expanding said cells to produce an NK cell population. The methods of the invention allow for the rapid expansion of NK cells, reducing the time needed for their culture, and hence the risk of exhaustion, enhancing the cytotoxicity of the NK cells when transfused into a patient.

When said method is carried out in vivo, said method is a therapeutic method as described herein. In such embodiments, all the disclosure herein in relation to therapeutic indications and applications of the invention is applicable to said methods.

As disclosed herein, the present inventors have shown that Notch ligands, particularly DLL4, can be used to enhance the production of NK cells. Accordingly, the invention provides an ex vivo method for expanding an NK cell population comprising: (a) culturing an HPC comprising sample obtained from an individual/patient in the presence of a Notch ligand, a fragment thereof which retains the function of said Notch ligand, or a molecule which mimics the function of said Notch ligand; and (b) culturing the cells produced by step (a) in the presence of IL-15; thereby producing an expanded NK cell population.

Step (a) and (b) may be carried out concurrently or in any order. For example, step (a) may be carried out first, followed by step (b), such that the cells are first exposed to a Notch ligand and then IL-15. Alternatively, step (b) may be carried out first, followed by step (a), such that the cells are first cultured in the presence of IL-15 and then in the presence of a Notch ligand. Alternatively, steps (a) and (b) may be carried out concurrently, such that the cells are simultaneously cultured in the presence of a Notch ligand and IL-15. Preferably step (a) is carried out first, followed by step (b).

The Notch ligand may be any Notch ligand (including functional fragments thereof and molecules which mimic the action/function/effect of the Notch ligand of interest) as defined herein. Typically the Notch ligand is DLL4, a fragment thereof which retains the function of DLL4, or a molecule which mimics the function of DLL4 (as defined herein).

Typically in step (a) the HPCs are cultured in the absence of exogenous IL-15, with exogenous Il-15 being added in step (b) only. As a non-limiting example, IL-15 may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. In some embodiments IL-15 is used at a concentration of about 50 ng/ml, about 40 ng/ml, about 35 ng/ml, about 30 ng/ml, about 25 ng/ml, about 20 ng/ml or about 10 ng/ml, preferably about 30 ng/ml.

Additional external stimuli, such as growth factors and/or cytokines, may be used to further enhance the production of NK cells. Said external stimuli may be present in step (a) and/or step (b) as appropriate. Non-limiting examples of suitable external stimuli include IL-7, Flt3L, stem cell factor (SCF), thrombopoietin (TPO), IL-3 and/or IL-6, or any combination thereof. In some preferred embodiments, IL-7, Flt3L and/or SCF, or any combination thereof is used. More preferably IL-7, Flt3L and SCF are used.

As a non-limiting example, IL-7 may be used at a concentration of about 1 ng/ml to about 100 ng/ml, about 1 ng/ml to about 50 ng/ml, about 1 ng/ml to about 25 ng/ml, about 1 ng/ml to about 10 ng/ml or less. In some embodiments IL-7 is used at a concentration of about 50 ng/ml, about 25 ng/ml, about 20 ng/ml, about 15 ng/ml, about 10 ng/ml or about 5 ng/ml, preferably about 10 ng/ml. As a non-limiting example, Flt3L may be used at a concentration of about 1 ng/ml to about 100 ng/ml, about 1 ng/ml to about 50 ng/ml, about 1 ng/ml to about 25 ng/ml, about 1 ng/ml to about 10 ng/ml or less. In some embodiments Flt3L is used at a concentration of about 50 ng/ml, about 25 ng/ml, about 20 ng/ml, about 15 ng/ml, about 10 ng/ml or about 5 ng/ml, preferably about 10 ng/ml. As a non-limiting example, SCF may be used at a concentration of about 1 ng/ml to about 200 ng/ml, about 1 ng/ml to about 150 ng/ml, about 1ng/ml to about 100 ng/ml, about 1 ng/ml to about 50 ng/ml or less. In some embodiments SCF is used at a concentration of about 150 ng/ml, about 125 ng/ml, about 120 ng/ml, about 110 ng/ml, about 100 ng/ml, about 90 ng/ml, about 80 ng/ml or about 75 ng/ml, preferably about 100 ng/ml.

Typically IL-7, Flt3L and SCF are used together in step (a) only, step (b) only or in both steps (a) and (b). In some preferred embodiments, the HPCs are cultured in the presence of IL-7, Flt3L and/or SCF, more preferably IL-7, Flt3L and SCF, in step (a).

The Notch ligand (such as DLL4) may be present in solution (e.g. in the culture medium) or used to coat the vessel in which the HPCs are cultured. Preferably the Notch ligand (e.g. DLL4) is used to coat the vessel in which the HPCs are cultured. As a non-limiting example, the Notch ligand (e.g. DLL4) may be used at a concentration of about 1 μg/ml to about 100 μg/ml, about 1 μg/ml to about 50 μg/ml, about 1 μg/ml to about 25 μg/ml, about 1 μg/ml to about 10 μg/ml or less. In some embodiments the Notch ligand (e.g. DLL4) is used at a concentration of about 50 μg/ml, about 25 μg/ml, about 20 μg/ml, about 15 μg/ml, about 10 μg/ml, or about 5 μg/ml, preferably about 10 μg/ml. Additional substrates and/or linkers may be used to facilitate the attachment of the Notch ligand (such as DLL4) to the surface of the culture vessels. Examples of such substrates are known in the art, such as poly-L-lysine.

Step (a) and/or step (b) of the method of the invention may involve culturing said cells in the presence or absence of a stromal support cell or feeder cell, or population thereof. As used herein, the terms stromal cell, feeder cell and stromal support cell are synonymous and may be used interchangeably. Examples of such support/feeder cells include, but are not limited to, OP9 cells and/or EL08-1D2 cells. Typically step (a) is carried out in the absence of a stromal support cell or population thereof. In some preferred embodiments, both step (a) and step (b) are carried out in the absence of a stromal support cell or population thereof. In other words, these steps may be carried out coating the Notch ligand of the invention (e.g. DLL4) directly onto tissue culture plastic.

Steps (a) and (b) may be of any appropriate duration to maximise the production of NK cells. As a non-limiting example, step (a) may involve the culture of HPCs in the presence of a Notch ligand (such as DLL4) for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week, i.e. step (a) may be any of these durations. Typically step (a) is 72 hours to 1 week in length. As a non-limiting example, step (b) may involve the culture of the cells produced by step (a) in the presence of IL-15 for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week, at least 2 weeks, at least 3 weeks or longer, until the desired number of NK cells is produced. Thus, step (b) may be any of these durations. Typically step (b) is 1 week or more in length, in some preferred embodiments step (b) is 7 to 9 days in length, in even more preferred embodiments, step (b) is about two weeks in length.

Alternatively, the duration of step (a) and/or step (b) may be measured in terms of the number of cell passages. For example, step (a) and/or step (b) may be at least one, at least two, at least three, at least four, at least five, at least ten, at least 20, at least 30, at least 40 or more passages of the cells (either in vivo, or cultured ex vivo or in vitro).

The durations of step (a) and step (b) are independent, and any duration of step (a) above may be used in combination with any duration of step (b) above. In some preferred embodiments, step (a) is 72 to 1 week in length and step (b) is 1 week (or more) in length.

The present inventors have also demonstrated that combining the use of a Notch ligand (such as DLL4) and REV-ERB inhibition results in a surprisingly potent means for enhancing NK cell production, allowing for the production of large numbers of functional NK cells that are suitable for in vivo therapeutic use more rapidly than the current methods. It is surprising that these two independent mechanisms (Notch ligand and REV-ERB inhibition) can be used together to demonstrate a greater increase in NK cell number than either mechanism provides alone.

Accordingly, the present invention provides in vitro, in vivo and ex vivo methods for expanding NK cells using the combination of a Notch ligand and REV-ERB inhibition. When said method is carried out in vivo, said method is a therapeutic method as described herein. In such embodiments, all the disclosure herein in relation to therapeutic indications and applications of the invention is applicable to said methods. Typically the method of the invention is ex vivo.

Thus, the invention provides an ex vivo method for expanding an NK cell population comprising the steps of: (a) culturing an HPC comprising sample obtained from an individual/patient with a compound that inhibits the action of REV-ERB (as described herein); (b) culturing said cells in the presence of a Notch ligand (such as DLL4); and (c) expanding said cells in vitro to produce an NK cell population. Step (a) and (b) may be carried out concurrently or in any order. For example, step (a) may be carried out first, followed by step (b), such that the cells are first exposed to a REV-ERB inhibitory compound and then cultured in the presence of a Notch ligand. Alternatively, step (b) may be carried out first, followed by step (a), such that the cells are first cultured in the presence of a Notch ligand and then in the presence of a REV-ERB inhibitory compound. Alternatively, steps (a) and (b) may be carried out concurrently, such that the cells are simultaneously cultured in the presence of a REV-ERB inhibitory compound and a Notch ligand.

In some preferred embodiments, step (a) may be carried out first, followed by step (b), such that the cells are first cultured in the presence of a REV-ERB inhibitory compound and then in the presence of a Notch ligand. Thus, in those embodiments the invention provides an ex vivo method for expanding an NK cell population comprising the steps of: (a) culturing an HPC comprising sample obtained from an individual/patient with a compound that inhibits the action of REV-ERB (as described herein); (b) culturing said cells in the presence of a Notch ligand (such as DLL4); and (c) expanding said cells in vitro to produce an NK cell population.

The compound may be any REV-ERB inhibitory compound of the invention as described herein. Typically said compound increases E4bp4 expression by decreasing REV-ERB activity as described herein. The Notch ligand may be any Notch ligand of the invention as described herein. In some preferred embodiments the REV-ERB inhibitory compound is SR8278. In some preferred embodiments the Notch ligand is DLL4, a functional fragment thereof or a molecule which mimics the activity/function of DLL4. In some particularly preferred embodiments, the REV-ERB inhibitory compound is SR8278 and the Notch ligand is DLL4, a functional fragment thereof or a molecule which mimics the activity/function of DLL4.

The Notch ligand (such as DLL4) may be present in solution (e.g. in the culture medium) or used to coat the vessel in which the HPCs are cultured. Preferably the Notch ligand (e.g. DLL4) is used to coat the vessel in which the HPCs are cultured. Any appropriate concentration of Notch ligand may be used. Non-limiting examples of suitable Notch ligand concentrations are described herein. In other words, these steps may be carried out coating the Notch ligand of the invention (e.g. DLL4) directly onto tissue culture plastic.

The HPCs may be cultured in the presence or absence of a stromal support cell or feeder cell, or population thereof. Any appropriate stromal cell may be used, including, but not limited to OP9 stromal cells and/or EL08-1D2 stromal cells. In some preferred embodiments, the cells are cultured in the absence of a stromal support cell or population thereof.

Alternatively and/or in addition, the HPCs may be cultured in the presence of cytokines and growth factors associated with the development of cells in the NK cell differentiation pathway, including factors required for HPCs growth and/or factors required for NK cell growth and/or differentiation. Non-limiting examples of such factors include IL-3, IL-7, Flt3L, stem cell factor (SCF), TPO, IL-3, IL-6, and/or IL-15, or any combination thereof. Any appropriate concentration of such factors may be used. Non-limiting examples of suitable concentrations of these factors are described herein.

In some embodiments, the ex vivo method comprises a single stage in which the HPCs in a sample obtained from an individual/patient are cultured, contacted with a compound of the invention and a Notch ligand and expanded to form an NK cell population, typically under substantially constant culture conditions (i.e. steps (a) and (b) of the method are carried out concurrently). Typically this involves incubating the HPCs with factors such as IL-3, IL-7, SCF, Flt3L and/or IL-15, preferably all of these factors. The HPCs may be cultured in the presence or absence of stromal cells/cell layer, such as EL08-1D2 stromal cells.

In some embodiments, the ex vivo method comprises two stages. The first is a lymphoid production stage, in which the HPCs in a sample obtained from an individual/patient are cultured. Typically this involves incubating the H PCs with cytokines and growth factors associated with lymphoid production, such as Flt3L, IL-7 and/or SCF. This stage may last for at least one, at least two, at least three, at least four, or more days. In some preferred embodiments, this stage lasts for two days.

This is followed by a stage of NK cell expansion. Typically this involves culturing the cells in cytokines and growth factors associated with NK cell development, such as IL-15, and may involve transferring the cultured HSCs to a suitable stromal (support) cell layer, such as OP9 stromal cells. The second stage lasts for the remainder of the ex vivo culture period (as defined herein). The culture medium may be changed as often as required during this second stage in order to facilitate NK cell expansion.

In some embodiments, the REV-ERB inhibitory compound of the invention is added in stage 1 (lymphoid production) and the Notch ligand in the second stage (NK cell expansion). In other embodiments, the Notch ligand is added in stage 1 (lymphoid production) and the REV-EB inhibitory compound in the second stage (NK cell expansion). In yet other embodiments, both the REV-ERB inhibitory compound and the Notch ligand added in the first stage (lymphoid production). In further embodiments, both the REV-ERB inhibitory compound and the Notch ligand added in the second (NK cell expansion phase). If the REV-ERB inhibitory compound and the Notch ligand added in the same stage (either stage 1 or stage 2), that stage may be further divided so that: (i) the REV-ERB inhibitory compound is added before the Notch ligand; or (ii) the Notch ligand is added before the REV-ERB inhibitory compound. Alternatively, the Notch ligand and REV-ERB inhibitory compound may be added simultaneously in the same stage.

Typically the REV-ERB inhibitory compound is added during the first stage, and the Notch ligand is added during the second stage, and preferably at the start of this second stage.

The HPC comprising sample may be cultured ex vivo for at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days or more. Typically said sample is cultured for at least 9 days in order to produce an expanded NK cell population. These culture periods are for the total culture period of the ex vivo method, i.e. if there are two stages, these periods are for the total (stage 1 plus stage 2).

The REV-ERB inhibitory compound of the invention may be added to the sample comprising HPCs within one week, within six days, within five days, within four days, within three days, within two days, within one day of isolating the HPCs in the sample, or on the same day as isolating the NK cell precursors. Typically this is the same day that the sample is obtained from the patient. Preferably the REV-ERB inhibitory compound of the invention is added to the sample within two days of isolating the HPCs in the sample, such as on the day of isolation of the HPCs. Most preferably the REV-ERB inhibitory compound of the invention is added to the sample two days post isolation of the HPCs.

The Notch ligand of the invention may be added to the sample comprising HPCs within one week, within six days, within five days, within four days, within three days, within two days, within one day of isolating the HPCs in the sample, or on the same day as isolating the NK cell precursors. Typically this is the same day that the sample is obtained from the patient. Preferably the Notch ligand of the invention is added to the sample within four days of isolating the HPCs in the sample, such as on day two following isolation of the HPCs. Most preferably the Notch ligand of the invention is added to the sample two or four days post isolation of the HPCs.

Preferred embodiments of the invention comprise (i) adding the REV-ERB inhibitory compound and the Notch ligand to the sample on the day of isolation of the HPCs; (ii) adding the REV-ERB inhibitory compound to the sample on the day of isolation of the HPCs and adding the Notch ligand to the sample on day two post isolation of the HPCs; or (iii) adding the REV-ERB inhibitory compound to the sample on day two post isolation of the HPCs and adding the Notch ligand to the sample on day four post isolation of the HPCs; with option (iii) being particularly preferred. As demonstrated in the Examples, these particular conditions maximise the synergy between the REV-ERB inhibition and the Notch ligand, and hence maximising the expansion of NK cells.

The method of the invention may further comprise modulating (increasing or decreasing the expression and/or activity of one or more additional gene and/or protein in the HPCs in order to enhance NK cell expansion. This modulation may be elicited by a compound of the invention, including the same compound of the invention as used to inhibit the activity of REV-ERB. Alternatively, one or more additional compounds may be used to modulate the expression and/or activity of the one or more additional gene and/or protein. Said modulation may occur directly or indirectly. Indirect modulation encompasses downstream effects caused by a compound of the invention inhibiting the activity of REV-ERB.

Any of the methods of the invention may be used on its own or in combination with other methods of the invention. For example, methods of the invention relating to inhibiting the action of REV-ERB and culturing HPCs in the presence of a Notch ligand may be combined with the invention relating to culturing in the presence of DLL4 or a functional fragment thereof (i.e. the Notch ligand is DLL4 or a DLL4 fragment) and in the presence of IL-15 and/or the invention relating to post-translational modification of E4bp4 in order to increase E4bp4 activity. Any combination of the methods as disclosed herein is envisaged by the present invention.

In all methods of the invention, the sample comprising HPCs obtained from an individual/patient may be a sample obtained from bone marrow, cord blood and/or peripheral blood. Thus, the sample may be a cord or peripheral blood sample, or a bone marrow sample or biopsy. The sample may be obtained from the individual who is to be treated with the NK cell population produced by a method of the invention (i.e. a patient). Alternatively, the sample is obtained from a healthy individual.

According to the present invention, a sample comprising HPCs is any sample from an individual which comprises a sufficient number of HPCs (as described herein), such that an expanded NK cell population can be obtained by contacting said sample with a compound according to the present invention. Typically the sample comprises HSCs. Preferably said sample is enriched for HSCs, such as a cord or peripheral blood sample or a bone marrow sample or biopsy as described herein.

A method of the invention may result in an increase in, the number of NK cells of at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control.

A method of the invention may accelerate the production of phenotypically mature NK cells. In other words, the method of the invention may reduce the time taken to arrive at a population of mature NK cells. A reduction in the run time of the method offers a further advantage over the conventional methods for NK cell expansion known in the art. As a non-limiting example, current clinical procedures for the expansion of NK cells can take more than two weeks to generate an NK cell population that comprises about 20% mature NK cells. In contrast, a method of the invention may achieve an equivalent population in 10 days or less, preferably in one week or less. A method of the invention may achieve a population of at least 40% mature NK cells, preferably at least 45%, at least 46%, at least 47%, at least 48%, or at least 49% mature NK cells, even more preferably at least 50% mature NK cells in three weeks or less, 20 days or less, 19 days or less, 18 days or less, 17 days or less, 16 days or less, 15 days or less, two weeks or less, 13 days or less, or 12 days or less. Preferably a method of the invention can achieve a population of at least 45% mature NK cells within two weeks or less.

Typically any ex vivo method of the present invention involves a final step to purify the expanded NK cell population. This ensures a pure population for therapeutic administration as described herein. Purification of the expanded NK cell population may be by any appropriate means. Standard cell purification methods are known in the art, such as cell sorting, including fluorescence-activated cell sorting (FACS) and magnetic-activated cell sorting (MACS). In some methods of the invention, particularly those involving the combination of a Notch ligand and a REV-ERB inhibitory compound, the % of NK cells in the final cell population may be very high (typically greater than 85%, preferably greater than 90%, more preferably greater than 95%, and may approach 100%). In such instances, a final purification step may optionally be omitted.

Methods of Expanding CD16⁺ NK Cells or Increasing the Number of CD16⁺ NK Cells

The invention also relates to the production of NK cell populations comprising increased numbers of CD16⁺ NK cells. In particular, the present inventors have demonstrated that culturing HPCs for a defined period of time (a first culture period) in a medium which does not induce differentiation surprisingly results in the production of an expanded NK cell population with increased number of CD16⁺ NK cells, particularly when said method comprises the use of a Notch ligand (e.g. DLL4), and optionally a compound which inhibits the activity of REV-ERB.

Thus, the present invention provides a method for producing an expanded population of CD16⁺ NK cells, comprising the steps of: (A) culturing an HPC comprising sample obtained from an individual in medium which does not induce differentiation of the HPCs for a first culture period to produce a pre-differentiation HPC population; (B) culturing the pre-differentiation HPC population in medium which induces differentiation of the HPCs to NK for a second culture period. Step (B) typically comprises both the differentiation of the HPCs in the pre-differentiation HPC population and expansion of the resulting NK cells. Thus, the present invention provides a method for producing an expanded population of CD16⁺ NK cells, comprising the steps of: (A) culturing an HPC comprising sample obtained from an individual in medium which does not induce differentiation of the HPCs for a first culture period to produce a pre-differentiation HPC population; and (B) culturing the pre-differentiation HPC population in medium which induces differentiation of the HPCs to NK for a second culture period, wherein NK cell expansion also occurs in the second culture period. The present invention also provides a method for producing an expanded population of CD16⁺ NK cells, comprising the steps of: (A) culturing an HPC comprising sample obtained from an individual in medium which does not induce differentiation of the HPCs for a first culture period to produce a pre-differentiation HPC population; and (B) culturing the pre-differentiation HPC population in medium which induces differentiation of the HPCs to NK for a second culture period; and (C) expanding said cells in vitro to produce an expanded NK cell population.

The invention further provides a method for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population; and (B) culturing the pre-differentiation HPC population in medium which induces differentiation of the HPCs to NK for a second culture period. Step (B) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population; (B) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population; and (B) culturing the pre-differentiation HPC population in medium which induces differentiation of the HPCs to NK for a second culture period; and (C) expanding said cells in vitro to produce an expanded NK cell population.

The inventors have surprisingly demonstrated that culturing HPCs for a first culture period of between about 2 days to about 8 days in a medium which does not induce differentiation, prior to differentiation of the pre-differentiation HPC population into NK cells, increases the number of CD16⁺ NK cells in the resulting expanded NK cell population. Thus, the present invention provides an ex vivo method for producing an expanded population of CD16⁺ NK cells, comprising the steps of: (A) culturing an HPC comprising sample obtained from an individual in medium which does not induce differentiation of the HPCs, wherein the culturing is for between about 2 days to about 8 days, to produce a pre-differentiation HPC population; (B) culturing the pre-differentiation HPC population in medium which induces differentiation of the HPCs to NK for a second culture period, wherein NK cell expansion also occurs in the second culture period. The present invention also provides a method for producing an expanded population of CD16⁺ NK cells, comprising the steps of: (A) culturing an HPC comprising sample obtained from an individual in medium which does not induce differentiation of the HPCs, wherein the culturing is for between about 2 days about 8 days, to produce a pre-differentiation HPC population; and (B) culturing the pre-differentiation HPC population in medium which induces differentiation of the HPCs to NK for a second culture period; and (C) expanding said cells in vitro to produce an expanded NK cell population.

The invention further provides a method for increasing the number of CD16⁺ NK cells in an expanded NK cell population, comprising the steps of: (A) culturing an HPC comprising sample obtained from an individual in medium which does not induce differentiation of the HPCs, wherein the culturing is for between about 2 days to about 8 days, to produce a pre-differentiation HPC population; (B) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (A) culturing an HPC comprising sample obtained from an individual in medium which does not induce differentiation of the HPCs, wherein the culturing is for between about 2 to about 8 days, to produce a pre-differentiation HPC population; and (B) culturing the pre-differentiation HPC population in medium which induces differentiation of the HPCs to NK for a second culture period; and (C) expanding said cells in vitro to produce an expanded NK cell population.

The first culture period is between about 2 days to about 8 days. Thus, the first culture period may be 2, 2.5, 3, 3.5, 4, 4.5, 5, 5.5, 6, 6.5, 7, 7.5 or 8 days or a period of any duration between 2 and 8 days. Preferably the first culture period may be between about 2 days to about 6 days (e.g. 2, 2.5, 3, 3.5, 4, 4.5, 5, 5.5 or 6 days or a period of any duration between 2 and 6 days, such as between about 4 to 6 days). More preferably the first culture period may be between about 2 days to about 4 days (e.g. 2, 2.5, 3, 3.5 or 4 days or a period of any duration between 2 and 4 days).

The first culture period is a period of time in which HPCs or an HPC comprising sample obtained from an individual is cultured in a medium which does not induce differentiation of the HPCs, typically wherein said medium does not induce differentiation of the HPCs to NK cells. Any culture medium suitable for the culture of HPCs may be used, provided that said medium does not induce the differentiation of the HPCs, typically to NK cells. Non-limiting examples of suitable media and factors for including in said media are described herein. This step of culturing HPCs or an HPC comprising sample obtained from an individual is culturing in a medium which does not induce differentiation of the HPCs, typically wherein said medium does not induce differentiation of the HPCs to NK cells is typically step (A) of a method of the invention.

As used herein, the term “pre-differentiation HPC population” is used to define a population of HPCs obtained by culturing HPCs or an HPC comprising sample obtained from an individual for a first culture period in a medium which does not induce differentiation of the HPCs, typically wherein said medium does not induce differentiation of the HPCs to NK cells. Said pre-differentiation HPC population may comprise other non-HPC cell types, provided that the HPCs are the predominant cell type (e.g. at least 50% of the cells are HPCs). Typically the pre-differentiation HPC population comprises less than 20%, less than 15%, less than 10%, less than 9%, less than 8%, less than 7%, less than 6%, less than 5%, less than 4%, less than 3%, less than 2%, less than 1%, less than 0.5% or fewer NK cells. Preferably the pre-differentiation HPC population comprises less than 2% NK cells, more preferably less than 1% NK cells. The numbers of HPCs and/or NK cells in the HPC comprising sample and/or the pre-differentiation HPC population can be determined by any appropriate technique, conventional examples include techniques such as FACS or flow cytometry.

Following the step of culturing an HPC comprising sample obtained from an individual in medium which does not induce differentiation of the HPCs for a first culture period (e.g. between about 2 days to about 8 days, such as between about 2 days to about 6 days, about 4 to about 6 days or about 2 days to about 4 days) to produce a pre-differentiation HPC population, an ex vivo method of the invention comprises a step of culturing the (pre-differentiation) HPCs or a pre-differentiation HPC population in a medium which induces differentiation of the (pre-differentiation) HPCs to NK cells for a second culture period.

The second culture period may be between about 10 days to about 30 days. Thus, the second culture period may be 10, 11, 12, 13, 14, 15, 16, 17, 18, 19, 20, 21, 22, 23, 24, 25, 26, 27, 28, 29 or 30 days or a period of any duration between 10 and 30 days, such as between 24 and 30 days. Preferably the second culture period may be between about 15 days to about 25 days (e.g. 15, 16, 17, 18, 19, 20, 21, 22, 23, 24 or 25 days or a period of any duration between 15 and 25 days). More preferably the second culture period may be between about 18 days to about 22 days (e.g. 18, 19, 20, 21 or 22 days or a period of any duration between 18 and 22 days). Most preferably, the second culture period is about 19 to 21 days, with about 20 days being particularly preferred.

Thus, the present invention provides a method for producing an expanded population of CD16⁺ NK cells, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days or about 2 days to about 4 days) to produce a pre-differentiation HPC population; and (B) culturing the pre-differentiation HPC population in medium which induces differentiation of the HPCs to NK, wherein the culturing is for between about 15 days to about 30 days (e.g. between about 18 days to about 22 days, such as about 20 days). Step (B) typically comprises both the differentiation of the HPCs in the pre-differentiation HPC population and expansion of the resulting NK cells. Thus, the present invention provides a method for producing an expanded population of CD16⁺ NK cells, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; (B) culturing the pre-differentiation HPC population in medium which induces differentiation of the HPCs to NK, wherein the culturing is for between about 15 days to about 30 days (e.g. between about 18 days to about 22 days, such as about 20 days), wherein NK cell expansion also occurs in the second culture period. The present invention also provides a method for producing an expanded population of CD16⁺ NK cells, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; and (B) culturing the pre-differentiation HPC population in medium which induces differentiation of the HPCs to NK, wherein the culturing is for between about 15 days to about 30 days (e.g. between about 18 days to about 22 days, such as about 20 days); and (C) expanding said cells in vitro to produce an expanded NK cell population.

The invention further provides a method for increasing the number of CD16⁺ NK cells in an expanded NK cell population, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days or about 2 days to about 4 days) to produce a pre-differentiation HPC population; and (B) culturing the pre-differentiation HPC population in medium which induces differentiation of the HPCs to NK, wherein the culturing is for between about 15 days to about 30 days (e.g. between about 18 days to about 22 days, such as about 20 days). Step (B) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; (B) culturing the pre-differentiation HPC population in medium which induces differentiation of the H PCs to NK, wherein the culturing is for between about 15 days to about 30 days (e.g. between about 18 days to about 22 days, such as about 20 days), wherein NK cell expansion also occurs in the second culture period. The present invention also provides a method for increasing the number of CD16⁺ NK cells in an expanded NK cell population, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; (B) culturing the pre-differentiation HPC population in medium which induces differentiation of the HPCs to NK, wherein the culturing is for between about 15 days to about 30 days (e.g. between about 18 days to about 22 days, such as about 20 days); and (C) expanding said cells in vitro to produce an expanded NK cell population.

The second culture period is a period of time in which (pre-differentiation) HPCs or a pre-differentiation HPC population is cultured in a medium which induces differentiation of the (pre-differentiation) HPCs, typically wherein said medium induces differentiation of the (pre-differentiation) HPCs to NK cells. Any culture medium suitable for the culture of pre-differentiation HPCs may be used, provided that said medium induces the differentiation of the (pre-differentiation) HPCs, typically to NK cells. Non-limiting examples of suitable media and factors for including in said media are described herein. This step of culturing (pre-differentiation) HPCs or a pre-differentiation HPC population in a medium which induces differentiation of the (pre-differentiation) HPCs cells for a second culture period, typically wherein said medium induces differentiation of the (pre-differentiation) HPCs to NK cells is typically step (B) of a method of the invention.

A method of the invention typically also comprises expansion of the (CD16⁺) NK cells. By expansion, it is meant that the number of (CD16⁺) NK cells is increased. The increase in number of (CD16⁺) NK cells may be as described herein.

Expansion of the (CD16⁺) NK cells may occur in parallel (i.e. at the same time) as the differentiation of the (pre-differentiation) HPCs to NK cells. In other words, steps (B) and (C) may be simultaneous, such that the second culture period includes both differentiation and expansion, as described herein. Thus, the present invention provides a method for producing an expanded population of CD16⁺ NK cells, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; and (B) culturing for a second culture period (e.g. wherein the culturing is for between about 15 days to about 30 days, such as between about 18 days to about 22 days, such as about 20 days) the pre-differentiation HPC population in medium which induces differentiation of the HPCs to NK and expanding said cells in vitro to produce an expanded NK cell population. The invention also provides a method for increasing the number of CD16⁺ NK cells in an expanded NK cell population, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; and (B) wherein the culturing is for culturing for a second culture period (e.g. between about 15 days to about 30 days, such as between about 18 days to about 22 days, such as about 20 days) the pre-differentiation HPC population in medium which induces differentiation of the HPCs to NK and expanding said cells in vitro to produce an expanded NK cell population.

Alternatively, expansion of the (CD16⁺) NK cells may occur after the step of culturing the (pre-differentiation) HPCs or a pre-differentiation HPC population in a medium which induces differentiation of the (pre-differentiation) HPCs to NK cells for a second culture period. Thus, the present invention provides an ex vivo method for producing an expanded population of CD16⁺ NK cells, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; (B) culturing the pre-differentiation HPC population in medium which induces differentiation of the HPCs to NK for a second culture period (e.g. wherein the culturing is for between about 15 days to about 30 days, such as between about 18 days to about 22 days, such as about 20 days); and subsequently (C) expanding said cells in vitro to produce an expanded NK cell population. The invention further provides an ex vivo method for increasing the number of CD16⁺ NK cells in an expanded NK cell population, comprising the steps of: (A) culturing an HPC comprising sample obtained from an individual in medium which does not induce differentiation of the HPCs for a first culture period (e.g. wherein the culturing is for between about 2 days to about 8 days, such as between about 2 days to about 6 days, between about 4 to 6 days, or about 2 days to about 4 days) to produce a pre-differentiation HPC population; (B) culturing the pre-differentiation HPC population in medium which induces differentiation of the HPCs to NK for a second culture period (e.g. wherein the culturing is for between about 15 days to about 30 days, such as between about 18 days to about 22 days, such as about 20 days); and subsequently (C) expanding said cells in vitro to produce an expanded NK cell population.

The expansion of the NK cells may comprise culturing the (CD16⁺) NK cells for a period may of between about 10 days to about 30 days. As discussed herein, this may be in parallel (simultaneous with) the differentiation step, or subsequent to the differentiation step. In either case, the expansion period may be 10, 11, 12, 13, 14, 15, 16, 17, 18, 19, 20, 21, 22, 23, 24, 25, 26, 27, 28, 29 or 30 days or a period of any duration between 10 and 30 days, such as between 24 and 30 days. Preferably the expansion period may be between about 15 days to about 25 days (e.g. 15, 16, 17, 18, 19, 20, 21, 22, 23, 24 or 25 days or a period of any duration between 15 and 25 days). More preferably the expansion period may be between about 18 days to about 22 days (e.g. 18, 19, 20, 21 or 22 days or a period of any duration between 18 and 22 days). Most preferably, the expansion period is about 19 to 21 days, with about 20 days being particularly preferred.

The expansion period is a period of time in which (CD16⁺) NK cells are cultured in a medium which induces their expansion. Any culture medium suitable for the culture of (CD16⁺) NK cells may be used, provided that said medium induces the expansion of (CD16⁺) NK cells. Non-limiting examples of suitable media and factors for including in said media are described herein. The expansion medium may be the same as the medium used for the second culture period, i.e. the same medium which induces differentiation of the (pre-differentiation) HPCs to NK cells. Typically expansion involves culturing the cells in cytokines and growth factors associated with NK cell development, such as IL-15, and may involve transferring the pre-differentiation HPCs or the NK cells to a suitable stromal (support) cell layer, such as OP9 or EL08 stromal cells, or a cell-free alternative support layer such as an ECM as described herein. The expansion step typically lasts for the remainder of the ex vivo culture period (as defined herein). The culture medium may be changed as often as required during this stage in order to facilitate NK cell expansion. Any REV-ERB inhibitory compounds, Notch ligands and/or compounds which alter the posttranslational modification of E4bp4 that are present in the culture medium before it is replaced may be administered again with the fresh culture medium, either at the same or different concentration. Preferably, the REV-ERB inhibitory compounds, Notch ligands and/or compounds which alter the posttranslational modification of E4bp4 used in step (A) and/or (B) are not present during the expansion phase.

The present invention relates to methods for expanding a (CD16⁺) NK cell population and/or for increasing the number of (CD16⁺) NK cells in an expanded NK cell population. Said method may be in vitro, in vivo or ex vivo. Typically the method of the invention is ex vivo.

Steps (A) and (B) (where expansion is comprised in step (B)) or steps (A) to (C) (when expansion is subsequent to step (B)) of the methods of the invention are typically carried out in order. In other words, the HPC comprising sample is typically first cultured in medium which does not induce differentiation of the HPCs (to NK cells) for a first culture period to produce a pre-differentiation HPC population, followed by culturing of the pre-differentiation HPC population in medium which induces differentiation of the HPCs to NK cells for a second culture period, which may be followed by a separate step of expanding the NK cells in vitro. As described herein, preferably the NK cell expansion step is simultaneous or concurrent with the step of culturing of the pre-differentiation HPC population in medium which induces differentiation of the HPCs to NK cells for a second culture period, i.e. step (B) and (C) are simultaneous/concurrent, or at least partially overlap.

The durations of step (A) and step (B) (where expansion is comprised in step (B)) or step (A), step (B) and step (C) (when expansion is subsequent to step (B)) are independent, and any duration of step (A) above may be used in combination with any duration of step (B) above and/or any duration of step (C) above.

An ex vivo method of the invention may be at least 12 days in length (including steps (A), (B) and an expansion step when this is not concurrent with step (B)). By way of non-limiting example, an ex vivo method of the invention may be at least 14, at least 16, at least 18, at least 22, at least 24, at least 26, at least 28, at least 30, at least 32, at least 34, at least 36, or more days in length. By way of further non-limiting example, an ex vivo method of the invention may be between about 12 to about 36 days in length, such as between about 12 to about 28 days, between about 12 to about 26 days, between about 12 to about 24 days, between about 12 to about 22 days, between about 12 to about 18 days, between about 12 to about 16 days, or between about 12 to about 14 days.

In all methods of the invention, the sample comprising HPCs obtained from an individual/patient may be a sample obtained from bone marrow, cord blood and/or peripheral blood. Thus, the sample may be a cord or peripheral blood sample, or a bone marrow sample or biopsy. The sample may be obtained from the individual who is to be treated with the NK cell population produced by a method of the invention (i.e. a patient). Alternatively, the sample is obtained from a healthy individual.

The sample comprising HPCs may be treated prior to its use in the present methods. By way of non-limiting example, the sample comprising the HPCs may be frozen once it has been obtained from the subject. Samples which are frozen will typically require thawing prior to use in a method of the invention. Following thawing a sample may require plating and/or an initial culture period to allow the HPCs to recover from freezing prior to commencing a method of the invention. This initial culture period may be referred to as a “recovery period”. Methods of the invention may include such a recovery period or may not include such a recovery period. The recovery period, when present, may be one week, up to six days, up to five days, up to four days, up to three days, up to two days, up to one day in duration. A recovery period may preferably be up to 2 days, i.e. up to 1 day, up to 12 hours, up to 8 hours in duration. If a recovery period is not comprised in a method of the invention, step (A) of said method may begin on the same day as the date the method commences.

Step (A) of a method of the invention may begin on the same day as the method commences (e.g. from thawing and plating the HPCs or isolating the HPCs in the sample). Step (A) of a method of the invention may begin within one week, within six days, within five days, within four days, within three days, within two days, within one day, or on the same day as the date the method commences (e.g. from the date of thawing and plating the HPCs, the date of isolating the HPCs in the sample, or on the same day as isolating the NK cell precursors). The date of isolating the HPCs and/or NK cell precursors is typically the same day that the sample is obtained from the patient.

Step (A) of a method of the invention, typically begins following a recovery period as defined herein. Thus, step (A) of a method of the invention may begin within one week, within six days, within five days, within four days, within three days, within two days, within one day, within 12 hours or within 8 hours of commencement of the method (e.g. from thawing and plating the HPCs, the date of isolating the HPCs in the sample, or isolating the NK cell precursors). Preferably, step (A) of a method of the invention may begin within two days, within one day, within 12 hours or within 8 hours of commencement of the method, more preferably about 1 day following commencement of the method.

According to the present invention, a sample comprising HPCs is any sample from an individual which comprises a sufficient number of HPCs (as described herein), such that an expanded (CD16⁺) NK cell population can be obtained by a method of the present invention. Typically the sample comprises HSCs. Preferably said sample is enriched for HSCs, such as a cord or peripheral blood sample or a bone marrow sample or biopsy as described herein.

A method of the invention may result in an increase in the number of CD16⁺ NK cells of at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control.

A method of the invention may result in an increase in the number of CD16⁺ NK cells in an expanded NK cell population compared with the number of CD16⁺ NK cells in a control expanded NK cell population. A method of the invention may result in an increase in the proportion of CD16⁺ NK cells in an expanded NK cell population compared with the number of CD16⁺ NK cells in a control expanded NK cell population. A control expanded NK cell population may be as described herein. A control expanded NK cell population may be an expanded NK cell population produced by a control (e.g.) conventional method. Typically, a control expanded NK cell population may be an expanded NK cell population produced by a corresponding method to those described herein, but wherein (i) the step of culturing the HPC comprising sample in medium which does not induce differentiation of the HPCs for a first culture period (i.e. step (A)) is omitted; or (ii) the step of culturing the HPC comprising sample in medium which does not induce differentiation of the HPCs is carried out for less than about 2 days or for more than about 8 days.

A method of the invention may result in an increase in the number or proportion of CD16⁺ NK cells in an expanded NK cell population by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control expanded NK cell population, or an expanded NK cell population produced by a control method. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more at least 2×, 2.5×, 3×, 4×, 5×, 6×, 7×, 8×, 9×, 10× or more compared with a control expanded NK cell population, or an expanded NK cell population produced by a control method.

A method of the invention produces an expanded (CD16⁺) NK cell population as described herein. Typically, a method of the invention produces an expanded (CD16⁺) NK cell population wherein at least 10% of the NK cells in the population are CD16⁺ NK cells, at least 15% of the NK cells in the population are CD16⁺ NK cells, at least 20% of the NK cells in the population are CD16⁺ NK cells, at least 25% of the NK cells in the population are CD16⁺ NK cells, at least 30% of the NK cells in the population are CD16⁺ NK cells, at least 35% of the NK cells in the population are CD16⁺ NK cells, at least 40% of the NK cells in the population are CD16⁺ NK cells, at least 45% of the NK cells in the population are CD16⁺ NK cells, at least 50% of the NK cells in the population are CD16⁺ NK cells, at least 60% of the NK cells in the population are CD16⁺ NK cells, at least 70% of the NK cells in the population are CD16⁺ NK cells, at least 80% of the NK cells in the population are CD16⁺ NK cells, up to 100% of the NK cells in the population are CD16⁺ NK cells. Preferably at least 15% of the NK cells are CD16⁺ NK cells, more preferably at least 20% of the NK cells are CD16⁺ NK cells, still more preferably at least 25% of the NK cells are CD16⁺ NK cells even more preferably at least 30% of the NK cells are CD16⁺ NK cells.

The methods of the invention produce increased numbers of CD16⁺ NK cells without the need for introduction of exogenous nucleic acid (such as by transduction or transfection). This is in contrast to conventional methods for expanding NK cells with increased CD16 expression, which require expression of an exogenous CD16 transgene to achieve even minimal increases in CD16 expression.

Accordingly, the invention provides method for the production of expanded CD16⁺ NK cells, and methods for increasing the number of CD16⁺ NK cells in an expanded NK cell population, wherein said method does not comprise a step of contacting with and/or introducing to the HPCs and/or NK cells an exogenous nucleic acid (typically a nucleic acid encoding for CD16).

A method of the invention may accelerate the production of phenotypically mature NK cells. In other words, the method of the invention may reduce the time taken to arrive at a population of mature NK cells. A reduction in the run time of the method offers a further advantage over the conventional methods for NK cell expansion known in the art.

An ex vivo method of the present invention may comprise one or more additional steps. By way of non-limiting example, an ex vivo method of the invention may comprise one or more further, initial, step(s) of isolating and/or enriching HPCs from a sample (e.g. a peripheral or cord blood sample). By way of further non-limiting example, an ex vivo method of the present invention may comprise a further, typically final, step to purify the expanded (CD16⁺) NK cell population. This ensures a pure population for therapeutic administration as described herein. Purification of the expanded NK cell population may be by any appropriate means. Standard cell purification methods are known in the art, such as cell sorting, including fluorescence-activated cell sorting (FACS) and magnetic-activated cell sorting (MACS).

In some methods of the invention no REV-ERB inhibitory compound is used in step (A) and/or in step (B). In some methods of the invention, no REV-ERB inhibitory compound is used in either step (A) or step (B). Any and all disclosure here in relation to methods of the invention applies equally and without reservation to methods in which no REV-ERB inhibitory compound is used, unless stated to the contrary.

Preferably a Notch ligand (e.g. DLL4) is used in step (A) and no Notch ligand (e.g. DLL4) is used in step (B). Any and all disclosure here in relation to methods of the invention applies equally and without reservation to methods in which no Notch ligand (e.g. DLL4) is used in step (B), unless stated to the contrary.

In some methods of the invention, including but not limited to those involving the combination of a Notch ligand and a REV-ERB inhibitory compound, the % of NK cells in the final cell population may be very high (typically greater than 85%, preferably greater than 90%, more preferably greater than 95%, and may approach 100%). In such instances, a final purification step may optionally be omitted.

### Methods Using Notch Ligand

The methods for producing an expanded population of CD16⁺ NK cells and/or the method for increasing the number of CD16⁺ NK cells in an expanded NK cell population involve culturing NK cell precursors (HPCs) in the presence of a Notch ligand as described herein (e.g. DLL4). A method may comprise contacting HPCs with the Notch ligand (e.g. DLL4). These methods of the invention allow for the rapid expansion of NK cells, reducing the time needed for their culture, and hence the risk of exhaustion, enhancing the cytotoxicity of the NK cells when transfused into a patient.

The HPCs are typically cultured in the presence of the Notch ligand (e.g. DLL4) for at least part of a first culture period in a method described herein. Thus, the Notch ligand (e.g. DLL4) may be comprised in the medium which does not induce differentiation of the HPCs. Accordingly, the HPCs may be contacted with (cultured in the presence of) a Notch ligand (e.g. DLL4) for at least part of step (A) of a method of the invention.

As described herein, the first culture period is between about 2 days to about 8 days. The HPCs may be contacted with a Notch ligand (e.g. DLL4) for at least part of the first culture period, up to the entirety of the first culture period. Thus, the HPCs may be contacted with a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a Notch ligand (e.g. DLL4) for between about 2 days to about 4 days (e.g. 2, 2.5, 3, 3.5 or 4 days or a period of any duration between 2 and 4 days) of the first culture period.

Most preferably, the first culture period comprises a period of between about 0 to about 2 days, particularly about 1 day, in which a Notch ligand (e.g. DLL4) is absent, followed by contacting the HPCs with the Notch ligand (e.g. DLL4) for the remainder of the first culture period. Thus, a Notch ligand may be added to the medium which does not induce differentiation of the HPCs after a period of between about 0 to about 2 days, particularly the Notch ligand may be added to the medium which does not induce differentiation of the HPCs after about 1 day.

A method of the invention may comprise a recovery period, such that the HPCs are culture for a recovery period of between about 0 days to about 2 days before being contacted with a Notch ligand (e.g. DLL4) and cultured for a first culture period. As described herein, the first culture period is between about 2 days to about 8 days. The HPCs may be contacted with a Notch ligand (e.g. DLL4) for at least part of the first culture period, up to the entirety of the first culture period. Thus, the H PCs may be contacted with a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as, between about 4 to 6 days) of the first culture period. More preferably the HPCs may be contacted with a Notch ligand (e.g. DLL4) for between about 2 days to about 4 days (e.g. 2, 2.5, 3, 3.5 or 4 days or a period of any duration between 2 and 4 days) of the first culture period. A method may comprise a recovery period of between about 0 to about 2 days, particularly about 1 day, in which a Notch ligand (e.g. DLL4) is absent, followed by contacting the HPCs with the Notch ligand (e.g. DLL4) for a first culture period. Thus, a Notch ligand may be added to the medium which does not induce differentiation of the HPCs after a period of between about 0 to about 2 days, particularly the Notch ligand may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise Notch ligand (e.g. DLL4), depending on whether the presence of the Notch ligand (e.g. DLL4) is desired at that point within the first culture period. By way of non-limiting example, if the medium which does not induce differentiation of the HPCs is replaced on day 2, but the HPCs are to be cultured with the Notch ligand (e.g. DLL4) only from day 4, then the replacement medium which does not induce differentiation of the HPCs at day 2 would not include the Notch ligand (e.g. DLL4). By way of further limiting example, alternatively or in addition, if the medium which does not induce differentiation of the HPCs is replaced on day 6, and the HPCs are to be cultured with the Notch ligand (e.g. DLL4) from day 4, then the replacement medium which does not induce differentiation of the HPCs at day 6 would include the Notch ligand (e.g. DLL4). The Notch ligand (e.g. DLL4) may be added to the medium which does not induce differentiation of the HPCs before it is used to replace that on the H PCs, or the medium which does not induce differentiation of the HPCs may be replaced, and then Notch ligand (e.g. DLL4) then added to the HPCs.

Alternatively or in addition, the pre-differentiation HPC population may be cultured in the presence of the Notch ligand (e.g. DLL4) for at least part of a second culture period in a method described herein. Thus, the Notch ligand (e.g. DLL4) may be comprised in the medium which does induce differentiation of the HPCs. Accordingly, the pre-differentiation HPCs may be contacted with (cultured in the presence of) a Notch ligand (e.g. DLL4) for at least part of step (B) of a method of the invention. This may be as an alternative or in addition to the inclusion of a Notch ligand (e.g. DLL4) in step (A) as described herein.

As described herein, the second culture period may be between about 10 days to about 30 days. The pre-differentiation HPCs may be contacted with a Notch ligand (e.g. DLL4) for at least part of the second culture period, up to the entirety of the second culture period. Thus, the pre-differentiation HPCs may be contacted with a Notch ligand (e.g. DLL4) for 10, 11, 12, 13, 14, 15, 16, 17, 18, 19, 20, 21, 22, 23, 24, 25, 26, 27, 28, 29 or 30 days of the second culture period, or a period of any duration between 10 and 30 days. Preferably the pre-differentiation HPCs may be contacted with a Notch ligand (e.g. DLL4) for between about 15 days to about 25 days (e.g. 15, 16, 17, 18, 19, 20, 21, 22, 23, 24 or 25 days or a period of any duration between 15 and 25 days) or between about 24 to about 30 days (e.g. 24, 25, 26, 27, 28, 29 or 30 days, or a period of any duration between 24 and 30 days) of the second culture period. More preferably the pre-differentiation HPCs may be contacted with a Notch ligand (e.g. DLL4) for between about 18 days to about 22 days (e.g. 18, 19, 20, 21 or 22 days or a period of any duration between 18 and 22 days) of the second culture period. Most preferably, the pre-differentiation HPCs may be contacted with a Notch ligand (e.g. DLL4) for between about 19 to 21 days of the second culture period, with about 20 days being particularly preferred.

Preferably a Notch ligand (e.g. DLL4) is present only before differentiation of the HPCs is induced. In other words, a Notch ligand (e.g. DLL4) is present only for at least part of the first culture period, and preferably not for at least part of the second culture period. Thus, a Notch ligand (e.g. DLL4) is preferably present in the medium which does not induce differentiation of the HPCs, and not the medium which does induce differentiation of the HPCs. The pre-differentiation HPC population resulting from step (A) of a method of the invention may be transferred to a different culture vessel prior to step (B). This reduces the risk of contamination of the Notch ligand in step (B) of the method.

Typically the Notch ligand is a Notch ligand as described herein. Preferably, the Notch ligand is DDL4, or a fragment thereof which retains the function of DLL4, as described herein.

The Notch ligand (such as DLL4) may be present in solution (e.g. in the culture medium) or used to coat the vessel in which the HPCs are cultured. Preferably the Notch ligand (e.g. DLL4) is used to coat the vessel in which the HPCs are cultured. Any appropriate concentration of Notch ligand may be used, such as described herein.

As described above, HPCs may be cultured in the presence or absence of a stromal support cell or feeder cell, or population thereof. In some preferred embodiments where a Notch ligand is used, the cells are cultured in the absence of a stromal support cell or population thereof, but optionally may be cultured with an ECM as described herein.

The Notch ligand of the invention may be added to the sample comprising HPCs within one week, within six days, within five days, within four days, within three days, within two days, within one day of commencing a method of the invention. For example, from the date of thawing and plating the HPCs, the date of isolating the HPCs in the sample, or on the same day as isolating the NK cell precursors. The date of isolating the HPCs and/or NK cell precursors is typically the same day that the sample is obtained from the patient. Preferably the Notch ligand of the invention is added to the sample within four days of commencing a method of the invention (e.g. from thawing and plating the HPCs or isolating the HPCs in the sample), such as on the day of commencement (day 0), or day one or two following commencement (e.g. from thawing and plating the HPCs or isolation of the HPCs). Most preferably the Notch ligand of the invention is added to the sample one day post commencement (e.g. from thawing and plating the HPCs or isolation of the HPCs). Thus, typically the Notch ligand is present on or from one day post commencement (e.g. from thawing and plating the HPCs or isolation of the HPCs), until the pre-differentiation HPC population is cultured in the medium which induces differentiation of the pre-differentiation HPCs to NK cells (i.e. from day one post commencement (e.g. from thawing and plating the HPCs or isolation) until the end of step (A)/start of step (B)).

The cells (e.g. the HPCs in step (A)) may be cultured in the presence of a Notch ligand (such as DLL4) for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 7 days, or 8 days. Typically for between 1-7 days, preferably for about 4 days.

Alternatively, these durations may be measured in terms of the number of cell passages. For example, at least one, at least two, at least three, at least four, at least five, at least ten, at least 20, or more passages of the cells (either in vivo, or cultured ex vivo or in vitro). Typically, these durations may be up to 10 passages of the cells, i.e. any number of passages between 1 and 10 (1, 2, 3, 4, 5, 6, 7, 8, 9 or 10 passages), preferably 1, 2 or 3 passages.

The present invention provides a method for producing an expanded population of CD16⁺ NK cells or for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and cultured in contact with the Notch ligand (e.g. DLL4) for 4 days; and (B) culturing the pre-differentiation HPC population in medium which induces differentiation of the HPCs to NK for a second culture period to allow both the differentiation of the HPCs in the pre-differentiation HPC population and expansion of the resulting NK cells. Wherein (i) the medium which does not induce differentiation of the HPCs does not contain a REV-ERB inhibitory compound; and (ii) the medium which induces differentiation of the HPCs to NK does not comprise either a REV-ERB inhibitory compound or a Notch ligand.

Methods Using Compounds which Inhibit REV-ERB Activity

As described herein, the methods of the invention may not comprise the use of a compound which inhibits REV-ERB activity. However, in some embodiments, the HPCs may be cultured in the presence of a compound which inhibits REV-ERB activity for at least part of a first culture period in a method described herein. Thus, the compound which inhibits REV-ERB activity may be comprised in the medium which does not induce differentiation of the HPCs. Accordingly, the HPCs may be contacted with (cultured in the presence of) a compound which inhibits REV-ERB activity for at least part of step (B) of a method of the invention.

As described herein, the first culture period is between about 2 days to about 8 days. The HPCs may be contacted with a compound which inhibits REV-ERB activity for at least part of the first culture period, up to the entirety of the first culture period. Thus, the HPCs may be contacted with a compound which inhibits REV-ERB activity for 2, 2.5, 3, 3.5, 4, 4.5, 5, 5.5, 6, 6.5, 7, 7.5 or 8 days of the first culture period may or a period of any duration between 2 and 8 days, such as between 3 to 8 days or between 2.5 to 8 days. Preferably the HPCs may be contacted with a compound which inhibits REV-ERB activity for between about 2 days to about 6 days (e.g. 2, 2.5, 3, 3.5, 4, 4.5, 5, 5.5 or 6 days or a period of any duration between 2 and 6 days, such as between 2.5 to 6 days, between 3 to 6 days, or between about 4 to 6 days) of the first culture period. More preferably the HPCs may be contacted with a compound which inhibits REV-ERB activity for between about 2 days to about 4 days (e.g. 2, 2.5, 3, 3.5 or 4 days or a period of any duration between 2 and 4 days, such as between about 2.5 to 4 days, or between about 3 to 4 days) of the first culture period.

Most preferably, the first culture period comprises a period of between about 0 to about 2 days, particularly about 1 day, in which a compound which inhibits REV-ERB activity is absent, followed by contacting the HPCs with the compound which inhibits REV-ERB activity for the remainder of the first culture period. Thus, a compound which inhibits REV-ERB activity may be added to the medium which does not induce differentiation of the HPCs after a period of between about 0 to about 2 days, particularly the compound which inhibits REV-ERB activity may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise compound which inhibits REV-ERB activity, depending on whether the presence of the compound which inhibits REV-ERB activity is desired at that point within the first culture period. By way of non-limiting example, if the medium which does not induce differentiation of the HPCs is replaced on day 2, but the HPCs are to be cultured with the compound which inhibits REV-ERB activity only from day 4, then the replacement medium which does not induce differentiation of the HPCs at day 2 would not include the compound which inhibits REV-ERB activity. By way of further limiting example, alternatively or in addition, if the medium which does not induce differentiation of the HPCs is replaced on day 6, and the HPCs are to be cultured with the compound which inhibits REV-ERB activity from day 4, then the replacement medium which does not induce differentiation of the HPCs at day 6 would include the compound which inhibits REV-ERB activity. The compound which inhibits REV-ERB activity may be added to the medium which does not induce differentiation of the HPCs before it is used to replace that on the HPCs, or the medium which does not induce differentiation of the HPCs may be replaced, and the compound which inhibits REV-ERB activity then added to the HPCs.

Alternatively or in addition, the pre-differentiation HPC population may be cultured in the presence of the compound which inhibits REV-ERB activity for at least part of a second culture period in a method described herein. Thus, the compound which inhibits REV-ERB activity may be comprised in the medium which does induce differentiation of the HPCs. Accordingly, the pre-differentiation HPCs may be contacted with (cultured in the presence of) a compound which inhibits REV-ERB activity for at least part of step (B) of a method of the invention. This may be as an alternative or in addition to the inclusion of a compound which inhibits REV-ERB activity in step (A) as described herein.

As described herein, the second culture period may be between about 10 days to about 30 days. The pre-differentiation HPCs may be contacted with a compound which inhibits REV-ERB activity for at least part of the second culture period, up to the entirety of the second culture period. Thus, the pre-differentiation HPCs may be contacted with a compound which inhibits REV-ERB activity for 10, 11, 12, 13, 14, 15, 16, 17, 18, 19, 20, 21, 22, 23, 24, 25, 26, 27, 28, 29 or 30 days of the second culture period, or a period of any duration between 10 and 30 days. Preferably the pre-differentiation HPCs may be contacted with a compound which inhibits REV-ERB activity for between about 15 days to about 25 days (e.g. 15, 16, 17, 18, 19, 20, 21, 22, 23, 24 or 25 days or a period of any duration between 15 and 25 days) of the second culture period. More preferably the pre-differentiation HPCs may be contacted with a compound which inhibits REV-ERB activity for between about 18 days to about 22 days (e.g. 18, 19, 20, 21 or 22 days or a period of any duration between 18 and 22 days) of the second culture period. Most preferably, the pre-differentiation HPCs may be contacted with a compound which inhibits REV-ERB activity for between about 19 to 21 days of the second culture period, with about 20 days being particularly preferred.

Preferably a compound which inhibits REV-ERB activity is present only before differentiation of the HPCs is induced. In other words, a compound which inhibits REV-ERB activity is present only for at least part of the first culture period, and preferably not for at least part of the second culture period. Thus, a compound which inhibits REV-ERB activity is preferably present in the medium which does not induce differentiation of the HPCs, and not the medium which does induce differentiation of the HPCs. The pre-differentiation HPC population resulting from step (A) of a method of the invention may be transferred to a different culture vessel prior to step (B). This reduces the risk of contamination of the compound which inhibits REV-ERB activity in step (B) of the method.

The REV-ERB inhibitor compound may be added before the addition of the Notch ligand, concurrently with the Notch ligand or after the Notch ligand is added. In some embodiments the compound is added at multiple time points, for example when the culture medium is changed. As a non-limiting example, the compound of the invention may be added one day into the first culture period, and then added again at day five of the first culture period.

The disclosure in relation to addition of a Notch ligand and/or a compound which inhibits the activity of REV-ERB applies independently to all methods of the invention, e.g. for methods which also use a compound which alters the posttranslational modification of E4bp4 as described herein.

Any appropriate concentration of a compound which inhibits the activity of REV-ERB may be used, provided that it inhibits the action of REV-ERB as described herein and has utility in expanding an NK cell population. As a non-limiting example, in any aspect of the invention, a compound which inhibits the activity of REV-ERB may be used at a final concentration of about 2 to about 20 μM, about 2 to about 15 μM, about 5 to about 15 μM, about 5 to about 14 μM, about 4 to about 13 μM, about 5 to about 12 μM, about 5 to about 11 μM, or preferably about 5 to about 10 μM.

As described above, HPCS may be cultured in the presence or absence of a stromal support cell or feeder cell, or population thereof. In some preferred embodiments where a compound which inhibits the activity of REV-ERB is used, the cells are cultured in the absence of a stromal support cell or population thereof, but optionally may be cultured with an ECM or stromal cells as described herein.

The REV-ERB inhibitory compound may be added to the sample comprising HPCs within one week, within six days, within five days, within four days, within three days, within two days, within one day of commencing a method of the invention. For example, from the date of thawing and plating the HPCS, the date of isolating the HPCS in the sample, or on the same day as isolating the NK cell precursors. The date of isolating the HPCs and/or NK cell precursors is typically the same day that the sample is obtained from the patient. Preferably the REV-ERB inhibitory compound is added to the sample within five days, more preferably two days of commencing a method of the invention (e.g. from thawing and plating the HPCs or isolating the HPCS in the sample), such as on the day of commencement (day 0), or day one or two following commencement (e.g. from thawing and plating the HPCs or isolation of the HPCs). Most preferably the REV-ERB inhibitory compound of the invention is added to the sample one day post commencement (e.g. from thawing and plating the HPCs or isolation of the HPCs).

Thus, typically the REV-ERB inhibitory compound is present on or from one day post commencement (e.g. from thawing and plating the HPCs or isolation of the HPCs), until the pre-differentiation HPC population is cultured in the medium which induces differentiation of the pre-differentiation HPCs to NK cells (i.e. from day one post commencement (e.g. from thawing and plating the HPCs or isolation) until the end of step (A)/start of step (B)).

The cells (e.g. the HPCs in step (A)) may be cultured in the presence of a REV-ERB inhibitory compound for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 7 days, or 8 days. Typically for between 1-7 days, preferably for about 4 days.

Alternatively, these durations may be measured in terms of the number of cell passages. For example, at least one, at least two, at least three, at least four, at least five, at least ten, at least 20, or more passages of the cells (either in vivo, or cultured ex vivo or in vitro). Typically, these durations may be up to 10 passages of the cells, i.e. any number of passages between 1 and 10 (1, 2, 3, 4, 5, 6, 7, 8, 9 or 10 passages), preferably 1, 2 or 3 passages.

A method of the invention may comprise a recovery period, such that the HPCs are culture for a recovery period of between about 0 days to about 5 days before being contacted with a REV-ERB inhibitory compound and cultured for a first culture period. As described herein, the first culture period is between about 2 days to about 8 days. The HPCs may be contacted with a REV-ERB inhibitory compound for at least part of the first culture period, up to the entirety of the first culture period. Thus, the HPCs may be contacted with a REV-ERB inhibitory compound for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a REV-ERB inhibitory compound for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a REV-ERB inhibitory compound for between about 2 days to about 4 days (e.g. 2, 2.5, 3, 3.5 or 4 days or a period of any duration between 2 and 4 days) of the first culture period. A method may comprise a recovery period of between about 0 days to about 5 days, such as about 0 day to about 2 days, particularly about 1 day, in which a REV-ERB inhibitory compound is absent, followed by contacting the HPCs with the REV-ERB inhibitory compound for a first culture period. Thus, a REV-ERB inhibitory compound may be added to the medium which does not induce differentiation of the HPCs after a period of between about 0 days to about 5 days, such as about 0 days to about 2 days, particularly the REV-ERB inhibitory compound may be added to the medium which does not induce differentiation of the HPCs after about 1 day.

Methods Using Both Notch Ligands and Compounds which Inhibit REV-ERB Activity

The methods of the invention may comprise the use of both a Notch ligand (e.g. DLL4) and a compound which inhibits the activity of REV-ERB. Any and all disclosure herein relating to methods comprising the use of Notch ligands (e.g. concentrations, timings, etc.) can be combined without limitation to any and all disclosure relating to method comprising the use of compounds which inhibit REV-ERB activity (e.g. concentrations, timings, etc.).

For at least part of the first culture period, the HPCs may also be contacted with (cultured in the presence of) a compound which inhibits REV-ERB activity and a Notch ligand (e.g. DLL4). Thus, the medium which does not induce differentiation of the HPCs may comprise a compound which inhibits REV-ERB activity and a Notch ligand (e.g. DLL4). Thus, the HPCs may be contacted with (cultured in the presence of) a compound which inhibits REV-ERB activity and a Notch ligand (e.g. DLL4) for at least part of step (A) of a method of the invention.

As described herein, the first culture period is between about 2 days to about 8 days. The HPCs may be contacted with a compound which inhibits REV-ERB activity and a Notch ligand (e.g. DLL4) for at least part of the first culture period, up to the entirety of the first culture period. The HPCs may be contacted with a compound which inhibits REV-ERB activity and a Notch ligand (e.g. DLL4) for different lengths of time within the first culture period, or for the same length of time. Thus, the HPCs may be contacted with a compound which inhibits REV-ERB activity and a Notch ligand (e.g. DLL4) for a length of time independently selected from 2, 2.5, 3, 3.5, 4, 4.5, 5, 5.5, 6, 6.5, 7, 7.5 or 8 days of the first culture period may or a period of any duration between 2 and 8 days for each of the compound which inhibits REV-ERB activity and the Notch ligand (e.g. DLL4). Preferably the HPCs may be contacted with a compound which inhibits REV-ERB activity and a Notch ligand (e.g. DLL4) for a length of time independently selected from between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period for each of the compound which inhibits REV-ERB activity and the Notch ligand (e.g. DLL4). More preferably the HPCs may be contacted with a compound which inhibits REV-ERB activity and a Notch ligand (e.g. DLL4) for a length of time independently selected from between about 2 days to about 4 days (e.g. 2, 2.5, 3, 3.5 or 4 days or a period of any duration between 2 and 4 days) of the first culture period for each of the compound which inhibits REV-ERB activity and the Notch ligand (e.g. DLL4).

Most preferably, the first culture period comprises a period of between about 0 to about 2 days, particularly about 1 day, in which a compound which inhibits REV-ERB activity and a Notch ligand (e.g. DLL4) are absent, followed by contacting the HPCs with the compound which inhibits REV-ERB activity and the Notch ligand (e.g. DLL4) for the remainder of the first culture period. Thus, a compound which inhibits REV-ERB activity and the Notch ligand (e.g. DLL4) be added to the medium which does not induce differentiation of the HPCs after a period of time selected independently for each of the compound which inhibits REV-ERB activity and the Notch ligand (e.g. DLL4) of between about 0 to about 2 days, particularly the compound which inhibits REV-ERB activity and the Notch ligand (e.g. DLL4) may be added to the medium which does not induce differentiation of the HPCs after about 1 day.

A method of the invention may comprise a recovery period, such that the HPCs are culture for a recovery period of between about 0 days to about 5 days before being contacted with a REV-ERB inhibitory compound and a Notch ligand (e.g. DLL4) and cultured for a first culture period. As described herein, the first culture period is between about 2 days to about 8 days. The HPCs may be contacted with a REV-ERB inhibitory compound and a Notch ligand (e.g. DLL4) for at least part of the first culture period, up to the entirety of the first culture period. Thus, the HPCs may be contacted with a REV-ERB inhibitory compound and a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a REV-ERB inhibitory compound and a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a REV-ERB inhibitory compound and a Notch ligand (e.g. DLL4) for between about 2 days to about 4 days (e.g. 2, 2.5, 3, 3.5 or 4 days or a period of any duration between 2 and 4 days) of the first culture period. A method may comprise a recovery period of between about 0 days to about 5 days, such as about 0 day to about 2 days, particularly about 1 day, in which a REV-ERB inhibitory compound and a Notch ligand (e.g. DLL4) are absent, followed by contacting the HPCs with the REV-ERB inhibitory compound and the Notch ligand (e.g. DLL4) for a first culture period. Thus, a REV-ERB inhibitory compound and a Notch ligand (e.g. DLL4) may be added to the medium which does not induce differentiation of the HPCs after a period of between about 0 days to about 5 days, such as about 0 days to about 2 days, particularly the REV-ERB inhibitory compound and the Notch ligand (e.g. DLL4) may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise a compound which inhibits REV-ERB activity and/or a Notch ligand (e.g. DLL4), depending on whether the presence of the compound which inhibits REV-ERB activity and/or the Notch ligand (e.g. DLL4) is desired at that point within the first culture period. By way of non-limiting example, if the medium which does not induce differentiation of the HPCs is replaced on day 2, but the HPCs are to be cultured with the compound which inhibits REV-ERB activity and/or the Notch ligand (e.g. DLL4) only from day 4, then the replacement medium which does not induce differentiation of the HPCs at day 2 would not include the compound which inhibits REV-ERB activity and/or the Notch ligand (e.g. DLL4). By way of further limiting example, alternatively or in addition, if the medium which does not induce differentiation of the HPCs is replaced on day 6, and the HPCs are to be cultured with the compound which inhibits REV-ERB activity and/or the Notch ligand (e.g. DLL4) from day 4, then the replacement medium which does not induce differentiation of the HPCs at day 6 would include the compound which inhibits REV-ERB activity and the Notch ligand (e.g. DLL4). The compound which inhibits REV-ERB activity and/or the Notch ligand (e.g. DLL4) may be added to the medium which does not induce differentiation of the HPCs before it is used to replace that on the HPCs, or the medium which does not induce differentiation of the HPCs may be replaced, and the compound which inhibits REV-ERB activity and/or the Notch ligand (e.g. DLL4) then added to the HPCs.

Alternatively or in addition, for at least part of the second culture period, the pre-differentiation HPCs may also be contacted with (cultured in the presence of) a compound which inhibits REV-ERB activity and a Notch ligand (e.g. DLL4). Thus the medium which does induce differentiation of the HPCs may comprise a compound which inhibits REV-ERB activity and a Notch ligand (e.g. DLL4). Thus, the pre-differentiation HPCs may be contacted with (cultured in the presence of) a compound which inhibits REV-ERB activity and a Notch ligand (e.g. DLL4) for at least part of step (B) of a method of the invention. This may be as an alternative or in addition to the inclusion of a compound which inhibits REV-ERB activity and/or a Notch ligand (e.g. DLL4) in step (A) as described herein.

As described herein, the second culture period may be between about 10 days to about 30 days. The pre-differentiation HPCs may be contacted with a compound which inhibits REV-ERB activity and a Notch ligand (e.g. DLL4) for at least part of the second culture period, up to the entirety of the second culture period. Thus, the pre-differentiation HPCs may be contacted with a compound which inhibits REV-ERB activity and a Notch ligand (e.g. DLL4) for a length of time independently selected from 10, 11, 12, 13, 14, 15, 16, 17, 18, 19, 20, 21, 22, 23, 24, 25, 26, 27, 28, 29 or 30 days of the second culture period, or a period of any duration between 10 and 30 days for each of the compound which inhibits REV-ERB activity and the Notch ligand (e.g. DLL4). Preferably the pre-differentiation HPCs may be contacted with a compound which inhibits REV-ERB activity and a Notch ligand (e.g. DLL4) for a length of time independently selected from between about 15 days to about 25 days (e.g. 15, 16, 17, 18, 19, 20, 21, 22, 23, 24 or 25 days or a period of any duration between 15 and 25 days) of the second culture period for each of the compound which inhibits REV-ERB activity and the Notch ligand (e.g. DLL4). More preferably the pre-differentiation HPCs may be contacted with a compound which inhibits REV-ERB activity and a Notch ligand (e.g. DLL4) for a length of time independently selected from between about 18 days to about 22 days (e.g. 18, 19, 20, 21 or 22 days or a period of any duration between 18 and 22 days) of the second culture period for each of the compound which inhibits REV-ERB activity and the Notch ligand (e.g. DLL4). Most preferably, the pre-differentiation HPCs may be contacted with a compound which inhibits REV-ERB activity and a Notch ligand (e.g. DLL4) for a length of time independently selected from between about 19 to 21 days of the second culture period, with about 20 days being particularly preferred for each of the compound which inhibits REV-ERB activity and the Notch ligand (e.g. DLL4).

Preferably a compound which inhibits REV-ERB activity and a Notch ligand (e.g. DLL4) are present only before differentiation of the HPCs is induced. In other words, a compound which inhibits REV-ERB activity and a Notch ligand (e.g. DLL4) are present only for at least part of the first culture period, and preferably not for at least part of the second culture period. Thus, a compound which inhibits REV-ERB activity and a Notch ligand (e.g. DLL4) are preferably present in the medium which does not induce differentiation of the HPCs, and not the medium which does induce differentiation of the HPCs. The pre-differentiation HPC population resulting from step (A) of a method of the invention may be transferred to a different culture vessel prior to step (B). This reduces the risk of contamination of the compound which inhibits REV-ERB activity and the Notch ligand (e.g. DLL4) in step (B) of the method.

When a compound which inhibits REV-ERB activity and a Notch ligand (e.g. DLL4) are used, these may be added concurrently or in any order. Thus, the cells may be first exposed to a REV-ERB inhibitory compound and then cultured in the presence of a Notch ligand. Alternatively, the cells may be first cultured in the presence of a Notch ligand and then in the presence of a REV-ERB inhibitory compound. Alternatively, the cells may be simultaneously cultured in the presence of a REV-ERB inhibitory compound and a Notch ligand. Preferably the cells are first cultured in the presence of a REV-ERB inhibitory compound and then in the presence of a Notch ligand.

In some embodiments, the REV-ERB inhibitory compound of the invention is added in step (A) and the Notch ligand in step (B). In other embodiments, the Notch ligand is added in step (A) and the REV-ERB inhibitory compound in step (B). In yet other embodiments, both the REV-ERB inhibitory compound and the Notch ligand added in step (A). In further embodiments, both the REV-ERB inhibitory compound and the Notch ligand are added in step (B). If the REV-ERB inhibitory compound and the Notch ligand added in the same stage (either step (Aa) or step (B)), that stage may be further divided so that: (i) the REV-ERB inhibitory compound is added before the Notch ligand; or (ii) the Notch ligand is added before the REV-ERB inhibitory compound. Alternatively, the Notch ligand and REV-ERB inhibitory compound may be added simultaneously in the same stage. Preferably the REV-ERB inhibitor are both added in step (A), with the REV-ERB inhibitor added first (e.g. at day 0 or on day 2), and the Notch ligand being added later (e.g. at day 2 or 4 respectively).

Preferred embodiments of the invention comprise (i) adding the REV-ERB inhibitory compound and the Notch ligand to the sample on day 1 post-commencement (e.g. from thawing and plating the HPCs or isolation of the HPCs); (ii) adding the REV-ERB inhibitory compound to the sample on the day of commencement (e.g. from thawing and plating the HPCs or isolation of the HPCs) and adding the Notch ligand to the sample on day 1 post-commencement (e.g. from thawing and plating the HPCs or isolation of the HPCs); or (iii) adding the REV-ERB inhibitory compound to the sample on day two post-commencement (e.g. from thawing and plating the HPCs or isolation of the HPCs) and adding the Notch ligand to the sample on day four post-commencement (e.g. from thawing and plating the HPCs or isolation of the HPCs); with option (ii) being particularly preferred. As demonstrated by the inventors, these particular conditions maximise the synergy between the REV-ERB inhibition and the Notch ligand, and hence maximising the expansion of NK cells.

The present invention provides a method for producing an expanded population of CD16⁺ NK cells or for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population, wherein (i) a REV-ERB inhibitory compound is added on day 1 post-commencement and (ii) following about 1 day in culture in contact with the REV-ERB inhibitory compound, the HPCs are transferred to a culture vessel coated with a Notch ligand (e.g. DLL4) for a further period of about 4 days; and (B) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

The present invention provides a method for producing an expanded population of CD16⁺ NK cells or for increasing the number of CD16⁺ NK cells in an expanded NK cell population, comprising the steps of: (A) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and (ii) following 4 days in culture in contact with the Notch ligand (e.g. DLL4), a REV-ERB inhibitory compound is added for a further period of about 24 hours; and (B) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

### Additional External Stimuli

Any of the methods herein, whether comprising the use of a Notch ligand and optionally a compound which inhibits REV-ERB activity may comprise the use of one or more additional external stimuli. Any and all disclosure herein relating to methods comprising the use of Notch ligands (e.g. concentrations, timings, etc.) and optionally compounds which inhibit REV-ERB activity (e.g. concentrations, timings, etc.) can be combined without limitation to any and all disclosure relating to method comprising the use of any external stimulus (e.g. concentrations, timings, etc.).

Additional external stimuli, such as growth factors and/or cytokines, may be used to further enhance the production of (CD16⁺) NK cells. Non-limiting examples of suitable external stimuli include IL-7, IL-15, Flt3L, stem cell factor (SCF), thrombopoietin (TPO), granulocyte-macrophage colony-stimulating factor (GM-CSF), IL-3 and/or IL-6, or any combination thereof. Any appropriate concentration of such factors may be used. Non-limiting examples of suitable concentrations of these factors are described herein.

The inventors have surprisingly found that omitting IL-3 from the medium which induces differentiation of the HPCs to NK, i.e. the medium used in the step of culturing the pre-differentiation HPC population in medium which induces differentiation of the HPCs to NK for a second culture period (step (B) of the methods of the invention) increases the number of CD16⁺ NK cells produced. As exemplified herein, the omission of IL-3 from the medium which induces differentiation of the HPCs to NK is particularly significant when the pre-differentiation HPC population is contacted with a Notch ligand (e.g. DLL4) in step (B), and/or the HPCs in step (A) were contacted with a Notch ligand (e.g. DLL4) in step (A). Preferably, therefore, wherein a Notch ligand (e.g. DLL4) is used in a method of the invention (in step (A) and/or (B) as described herein), IL-3 is omitted from step (B) of the method. Alternatively or in addition, wherein a method of the invention (in step (A) and/or (B) as described herein) involves the use of stromal cells, IL-3 is omitted from step (B) of the method.

Accordingly, a method of the invention may omit IL-3 from: (i) the medium which induces differentiation of the HPCs to NK, i.e. the medium used in the step of culturing the pre-differentiation HPC population in medium which induces differentiation of the HPCs to NK fora second culture period (step (B) of the methods of the invention); and/or (ii) the medium which does not induce differentiation of the HPCs for a first culture period to produce a pre-differentiation HPC population, i.e. the medium used in the step of culturing an HPC comprising sample obtained from an individual in medium which does not induce differentiation of the HPCs for a first culture period to produce a pre-differentiation HPC population (step (A) of the methods of the invention). Preferably IL-3 is omitted from the medium which induces differentiation of the HPCs to NK, i.e. the medium used in the step of culturing the pre-differentiation HPC population in medium which induces differentiation of the HPCs to NK for a second culture period (step (B) of the methods of the invention); but not from the medium which does not induce differentiation of the HPCs for a first culture period to produce a pre-differentiation HPC population, i.e. the medium used in the step of culturing an HPC comprising sample obtained from an individual in medium which does not induce differentiation of the HPCs for a first culture period to produce a pre-differentiation HPC population (step (A) of the methods of the invention).

In a step of culturing an HPC comprising sample obtained from an individual in medium which does not induce differentiation of the HPCs for a first culture period to produce a pre-differentiation HPC population, the medium may comprise one or more of GM-CSF, IL-3, IL-6, Flt3, TPO and/or SCF, or any combination thereof. Thus, step (A) of a method of the invention may comprise culturing an HPC comprising sample obtained from an individual in medium which does not induce differentiation of the HPCs for a first culture period to produce a pre-differentiation HPC population, wherein said medium comprises one or more of GM-CSF, IL-3, IL-6 Flt3, TPO and/or SCF, or any combination thereof. Preferably the medium which does not induce differentiation of the HPCs comprises GM-CSF, IL-3, IL-6 Flt3, TPO and SCF. Exemplary concentrations of these external stimuli are described herein. Typically in step (A) the HPCs are cultured in the absence of exogenous IL-15, with exogenous Il-15 being added in step (B) only.

In a step of culturing the pre-differentiation HPC population in medium which induces differentiation of the HPCs to NK for a second culture period, the medium may comprise one or more of IL-7, IL-15, Flt3, and/or SCF, or any combination thereof. Thus, step (B) of a method of the invention may comprise culturing the pre-differentiation HPC population in medium which induces differentiation of the HPCs to NK for a second culture period, wherein said medium comprises one or more of IL-7, IL-15, Flt3, and/or SCF, or any combination thereof. Preferably the medium which does induce differentiation of the HPCs comprises IL-7, IL-15, Flt3, and SCF. Exemplary concentrations of these external stimuli are described herein. Typically the medium which induces differentiation of the HPCs to NK cells does not comprise IL-3.

IL-7, Flt3L, SCF, IL-15 may each independently be used at any appropriate concentration, such as those described herein.

As a non-limiting example, TPO may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml.

As a non-limiting example, GM-CSF may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml.

As a non-limiting example, IL-3 may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml.

As a non-limiting example, IL-6 may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml.

The HPCs and/or the pre-differentiation HPC population may be cultured on or with suitable support/stromal cells or cell layer. Any appropriate stromal cell may be used, including, but not limited to OP9 stromal cells and/or EL08-1D2 stromal cells. Alternatively, the HPCs and/or the pre-differentiation HPC population may be cultured in the absence of support/stromal cells or cell layer. In other words step (A) and/or step (B) of a method of the invention may be carried out in the absence of a stromal/support cell or stromal/support cell layer. When no support/stromal cells are used, the HPCs and/or the pre-differentiation HPC population may be cultured on an extracellular matrix (ECM) or ECM protein. An ECM protein may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of defined and/or xeno-free ECM protein that may be useful in the matrix for cell culture include laminin, collagen I, collagen IV, fibronectin and vitronectin. The ECM composition may include synthetically generated peptide fragments of fibronectin or recombinant fibronectin, or a mixture of at least fibronectin and vitronectin.

One or more steps of a method of the invention, or the entirety of said method may be carried out in “xeno-free (XF)” or “animal component-free (ACF)” or “animal free” conditions. The terms XF and ACF when used in relation to a medium, an extracellular matrix, or a culture condition, refers to a medium, an ECM, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components. The media used in the methods of the invention (in steps (A) and/or (B)) may be “serum-free”, which refers to media with no unprocessed or unpurified serum, and accordingly can include media with purified blood-derived components or animal tissue-derived components (such as growth factors). The medium according to the present invention may contain or may not contain any alternatives to serum.

One or more steps of a method of the invention, or the entirety of said method may be carried out in “defined” conditions. The term “defined”, when used in relation to a medium, an extracellular matrix, or a culture condition, refers to a medium, an extracellular matrix, or a culture condition in which the nature and amounts of approximately all the components are known. A “chemically defined medium” refers to a medium in which the chemical nature of approximately all the ingredients and their amounts are known. These media are also called synthetic media.

Any methods of the invention, whether comprising the use of a Notch ligand and optionally a compound which inhibits REV-ERB activity, may comprise the use of one or more compound which results in the alteration of post-translational modification of E4bp4, thereby causing an increase in E4bp4 activity, as described herein. Optionally the alteration of post-translational modification of E4bp4 is a reduction in SUMOylation and/or phosphorylation of E4bp4 as described herein. In some preferred embodiments the compound which results in the alteration of post-translational modification of E4bp4: reduces SUMOylation at one or more of residues K10, K116, K219, K337 and/or K394 of E4bp4, or a residue corresponding thereto, or any combination thereof; and/or reduces phosphorylation at one or more of residues 5286, 5301 and/or S454 of E4bp4, or a residue corresponding thereto, or any combination thereof.

Any appropriate concentration of a compound which results in the alteration of post-translational modification of E4bp4 may be used, provided that it increases the activity of E4bp4 as described herein and has utility in expanding a (CD16⁺) NK cell population. As a non-limiting example, in any aspect of the invention, a compound which results in the alteration of post-translational modification of E4bp4 may be used at a final concentration of about 0.1 to about 20 μM, about 0.1 to about 15 μM, about 0.5 to about 15 μM, about 0.5 to about 14 μM, about 0.5 to about 12 μM, about 0.5 to about 11 μM, or about 0.5 to about 10 μM, such as from about 5 to about 10 μM. In some preferred embodiments, a compound which results in the alteration of post-translational modification of E4bp4 may be used at a final concentration of about 0.5 to about 5 μM, more preferably of about 0.5 to about 2 μM, even more preferable of about 0.5 to about 1 μM.

The REV-ERB inhibitor compound, Notch ligand, compound which alters E4bp4 post-translational modification and/or other external stimuli may be used simultaneously, separately or sequentially as described herein.

Each of the REV-ERB inhibitor compound, Notch ligand compound which alters E4bp4 post-translational modification and/or other external stimuli may independently be used as a single treatment or application or in multiple treatments or applications (in both in vitro, ex vivo or in vivo methods as described herein). For multiple applications, at least two, at least three, at least four, at least five, at least six, at least seven, at least eight, at least nine or more applications may be used. The multiple applications may be applied at any appropriate time points according to a method or treatment of the invention. By way of non-limiting example, a REV-ERB inhibitory compound of the invention, a Notch ligand, a compound which alters E4bp4 post-translational modification and/or other external stimuli may each independently be applied twice a day, once daily, every other day, once every three days or weekly. Typically the REV-ERB inhibitory compounds of the invention, the Notch ligand, the compound which alters E4bp4 post-translational modification and/or other external stimuli may independently be applied as necessary when the culture medium is changed.

The method of the invention may further comprise modulating (increasing or decreasing the expression and/or activity of one or more additional gene and/or protein in the HPCs in order to enhance (CD16⁺) NK cell expansion. This modulation may be elicited by a compound of the invention, including the same compound of the invention as used to inhibit the activity of REV-ERB. Alternatively, one or more additional compounds may be used to modulate the expression and/or activity of the one or more additional gene and/or protein. Said modulation may occur directly or indirectly. Indirect modulation encompasses downstream effects caused by a compound of the invention inhibiting the activity of REV-ERB.

### Therapeutic Indications

The invention provides products containing a compound which inhibits the action of REV-ERB and a Notch ligand as a combined preparation for simultaneous, separate or sequential use in a method of therapy by increasing the production of NK cells in a patient.

The Notch ligand for use in said method of therapy may be any Notch ligand as described herein. In some preferred embodiments, the Notch ligand is DLL4 or a fragment thereof which retains the function of DLL4.

The REV-ERB antagonist for use in said method of therapy may be any REV-ERB antagonist as described herein. Typically the REV-ERB antagonist for use in said method increases E4bp4 expression by decreasing REV-ERB activity.

A reference herein to products of the invention is a reference to the combination of a REV-ERB antagonist and Notch ligand as described herein (for simultaneous, separate or sequential use).

Any REV-ERB antagonist and any Notch ligand of the invention may be used in combination. As a non-limiting example, DLL4 or a functional fragment thereof may be used in combination with SR8278, ARN5187, ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine or 1-(4-fluorophenyl)-N-(3-((1-methylpiperidin-4-yl)methyl)benzyl)cyclopentan-1-amine, preferably SR8278.

Typically the method of therapy comprises administering the products (as described herein) to a patient or subject. The Notch ligand and REV-ERB antagonist may be administered simultaneously, separately or sequentially. For separate or sequential administration, the Notch ligand may be administered first, followed by the REV-ERB antagonist, or vice versa.

Sequential administration may mean that the two products are administered immediately one after the other, or that the second product is administered within 1 minute, within two minutes, within three minutes, within four minutes, within five minutes, within 10 minutes, within 15 minutes, within 20 minutes, within 25 minutes, within 30 minutes, within 45 minutes, within one hour, or more of the first product being administered.

Separate administration may mean that the second product is administered within one hour, within two hours, within three hours, within six hours, within 12 hours, within 24 hours, within 2 days, within 3 days, within 4 days, within 5 days, within 6 days, within 7 days or more of the first product being administered.

The invention also provides for the production of CD16⁺ NK cells and expanded NK cell populations with increased numbers of CD16⁺ NK cells. Also as described herein, CD16⁺ NK cells are typically more active than CD16⁻ or CD16^{lo} cells. In particular, CD16⁺ NK cells and expanded NK cell populations made by the methods of the present invention typically exhibit at least 50% greater ADCC, preferably at least 70% greater ADCC, compared with control NK cells as described herein.

As such, the invention further relates to methods of treatment comprising administering a therapeutically effective amount of CD16⁺ NK cells, expanded (CD16⁺) NK cell populations and/or compositions comprising the same to a patient in need thereof.

Typically the method of therapy comprises administering the products (as described herein) to a patient or subject.

Administration of CD16⁺ NK cells, expanded (CD16⁺) NK cell populations and/or compositions according to the invention to a patient typically increases the number of NK cells in said patient.

As used herein, the term “increasing the number of NK cells” and “increasing production of NK cells” can be understood to mean that the compound or products of the invention elicit(s) a significant increase in the number of NK cells in a patient. This increase in NK cell number may be measured relative to a control (as described herein in the context of increasing E4bp4 expression and inhibiting REV-ERB activity).

A reference to an increase in the number of NK cells and/or increasing NK cell production may be quantified in terms of a fold increase relative to a control. Typically a compound of the invention can increase the number of NK cells, or give rise to an increase in NK cell production, of at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 3 fold or more relative to a control.

Alternatively, a reference to increasing the number of NK cells and/or increasing NK cell production may be understood to mean that, the number of NK cells is increased by at least 10%, at least 20%, at least 30%, at least 40%, at least 50%, at least 60, at least 70%, at least 80%, at least 85%, at least 90%, at least 95%, at least 100%, at least 150%, at least 200%, at least 300% or more compared with the control. Typically the number of NK cells is increased by at least 50%, preferably at least 70%, more preferably at least 80%, even more preferably at least 90% or more compared with a control.

In some embodiments, an increase in the number of NK cells and/or increase in NK cell production may be defined in terms of the absolute number of NK cells in a sample or patient, such as the percentage of NK cells, for example the percentage of NK cells in the circulating lymphocyte population. For example, a compound of the invention may cause an increase in NK number, resulting in a percentage of NK cells of at least 10%, at least 15%, at least 20%, at least 25%, at least 30%, at least 35%, at least 40%, at least 45%, at least 50%, at least 55%, at least 60%, at least 65%, at least 70%, at least 75%, at least 80% or more.

The number of NK cells may be determined by quantitative and/or qualitative analysis, and may be measured directly or indirectly. The number of NK cells relative to a control may be determined using any appropriate technique. Suitable standard techniques, such as flow cytometry, FACS and MACS, are known in the art.

The number of NK cells may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week, at least 2 weeks, at least 3 weeks, at least 1 month or more. Typically this is assessed relative to the last administration of the compound which inhibits REV-ERB activity and/or the Notch ligand.

The number of NK cells may be quantified in terms of the total number of NK cells in a sample from an individual/patient or culture sample (from an ex vivo method of the invention).

In the context of the therapeutic uses and methods of the invention, a “subject” or “patient” (these terms are used interchangeably herein) is any animal patient that would benefit from an increase in the number of NK cells. Typical animal patients are mammals, such as primates. Preferably the patient is a human.

Thus, the present invention provides a method of treatment by increasing the number of NK cells in a patient in need thereof, comprising administering to said patient a therapeutically effective amount of a compound which inhibits the action of REV-ERB (as described herein) and a Notch ligand (as described herein).

Additionally, the present invention provides the use of a compound which inhibits the action of REV-ERB and a Notch ligand in the manufacture of a medicament. Said medicament increases the number of NK cells in a patient.

The therapeutic use or method of the invention may comprise administering a therapeutically effective amount of a compound or products of the invention (as defined herein), either alone or in combination with other therapeutic agents, to a subject or individual.

As used herein, the term “treatment” or “treating” embraces therapeutic or preventative/prophylactic measures.

The compounds or products of the invention may also be used as a preventative therapy. As used herein, the term “preventing” includes preventing the onset of symptoms associated with a disease or disorder that may be treated by increasing NK cell number and/or reducing the severity or intensity of said symptoms. The term “preventing” includes inducing or providing protective immunity against such diseases or disorders, particularly infectious diseases as described herein. Immunity may be quantified using any appropriate technique, examples of which are known in the art.

A compound or products of the invention may be administered to a patient already having a disease or disorder which may be treated by increasing NK cell number. For example, the patient may be suspected of having an infectious disease or cancer as described herein, and may or may not be showing symptoms of said disease or disorder. When administered to such a patient, a compound or products of the invention can cure, delay, reduce the severity of, or ameliorate one or more symptoms, and/or prolong the survival of a subject beyond that expected in the absence of such treatment.

Alternatively, a compound or products of the invention may be administered to a patient who may ultimately be infected with a particular infectious disease, or develop a disease or disorder as described herein, in order to cure, delay, reduce the severity of, or ameliorate one or more symptoms, and/or prolong the survival of a subject beyond that expected in the absence of such treatment, or, in the case of infectious diseases help prevent that patient from transmitting said disease.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects.

The invention relates to the treatment of any disease or disorder which may be beneficially treated with by increasing the number of NK cells in a patient. Such diseases and disorders include cancer, infectious diseases (acute and chronic), autoimmune diseases and diseases or disorders related to female infertility or pregnancy. Infectious diseases that may be treated according to the present invention include viral infection, and infection by other pathogens, including bacteria, protists, fugal, or helminth pathogens. Typically said pathogens are intracellular pathogens which have at least one intracellular phase in their life cycle. Infections of particular interest include viral infections, and zoonotic infections that are of particular importance from a public health perspective. Cancers that may be treated according to the present invention include bladder cancer, blood cancers, leukaemia, bone cancers, bowel cancer, brain tumours, breast cancer, kidney cancer, liver cancer, lung cancer, melanoma, ovarian cancer, pancreatic cancer, prostate cancer, skin cancer, stomach cancer, testicular cancer and uterine cancer. Autoimmune diseases that may be treated according to the present invention include systemic lupus erythematosus, rheumatoid arthritis, multiple sclerosis and obesity-induced insulin resistance. As used herein, the term diseases or disorders related to female infertility or pregnancy includes, but is not limited to, fetal growth restriction, preterm labour, defects in uterine vascular remodelling and preeclampsia.

The compounds or products of the invention may be used in combination with one or more additional therapeutic agents or treatments, which typically may be selected from a conventional treatment for the disease or disorder to be treated. As a non-limiting example, if a compound or products of the invention are for use in the treatment of a cancer, such as lung cancer, then said compound or products may be used in combination with conventional treatments for lung cancer, such as radiotherapy, chemotherapy or surgery. When used in combination with one or more additional therapeutic agent or treatment, a compound or products of the invention may be administered before, simultaneously with, or after the administration of the one or more additional therapeutic agent or treatment.

In some preferred embodiments, a compound or products of the invention is for use in combination with antibody-mediated immunotherapy. Antibody-mediated immunotherapy involves the administration of antibodies to a patient to target disease-specific antigens. Such antibodies could be used to increase the specificity and killing activity of NK cells, which express receptors for the Fc regions of IgG antibodies. Activation of these Fc receptors, leads to NK cell activation, resulting in cytokine secretion and release of cytotoxic granules by the activated NK cell, causing lysis of the cell expressing the disease antigen. Such combination therapy is particularly preferred for the treatment of cancer (using antibodies to tumour-specific antigens). Any antibody used in immunotherapy may be used in combination with a compound or products of the invention. Non-limiting examples of such antibodies include anti-CD20 mAbs (non-Hodgkin's lymphoma, chronic lymphocytic lymphoma), anti-ganglioside D2 (anti-GD2) mAbs (neuroblastoma, melanoma), anti-human epidermal growth factor (anti-HER2) mAbs (breast and gastric cancers), anti-epidermal growth factor receptor (anti-EGFR) mAbs (colorectal and head and neck cancer).

The invention also provides the use of a compound which results in the post-translational modification of E4bp4, thereby causing an increase in E4bp4 activity (as described herein) in a therapeutic use or method as described herein. Any and all of the disclosure herein in relation to therapeutic indications of a compound or products of the invention may apply equally and independently to therapeutic applications of compounds which result in the post-translational modification of E4bp4, according to the present invention. As a non-limiting example, the present invention provides a compound which results in the post-translational modification of E4bp4, thereby causing an increase in E4bp4 activity (as described herein) for use in a method of therapy, for example in the treatment of cancer, an infectious diseases, an autoimmune disease or a disease or disorder related to female infertility or pregnancy. As another non-limiting example, the invention provides a method of treatment by increasing the number of NK cells in a patient in need thereof, comprising administering to said patient a therapeutically effective amount of a compound which results in the post-translational modification of E4bp4, thereby causing an increase in E4bp4 activity.

In other aspects, the invention provides the use of an expanded NK cell population (as described herein) in a therapeutic use or method as described herein. Any and all of the disclosure herein in relation to therapeutic indications of a compound or products of the invention may apply equally and independently to therapeutic applications of the expanded NK cell populations of the invention. As a non-limiting example, the present invention provides an expanded NK cell population (as described herein) for use in a method of therapy, for example in the treatment of cancer, an infectious diseases, an autoimmune disease or a disease or disorder related to female infertility or pregnancy. As another non-limiting example, the invention provides a method of treatment by increasing the number of NK cells in a patient in need thereof, comprising administering to said patient a therapeutically effective amount of an expanded NK cell population.

### Pharmaceutical Compositions and Formulations

The terms “compound” or “products” are herein used interchangeably with the terms “therapeutic/prophylactic composition”, “formulation” or “medicament”.

The compound, products or expanded NK cell population of the invention (as defined above) can be combined or administered in addition to a pharmaceutically acceptable carrier, diluent and/or excipient. Alternatively or in addition the compound, products or expanded NK cell population of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations is generally by conventional routes e.g. intravenous, subcutaneous, intraperitoneal, or mucosal routes. The administration may be by parenteral injection, for example, a subcutaneous, intradermal or intramuscular injection. For example, formulations comprising antibodies or expanded NK cell populations of the invention may be particularly suited to administration intravenously, intramuscularly, intradermally, or subcutaneously. Administration of small molecule REV-ERB inhibitors may be injection, such as intravenously, intramuscularly, intradermally, or subcutaneously, or by oral administration (small molecules with molecule weight of less than 500 Da typically exhibiting oral bioavailability).

Accordingly, immunogenic compositions, therapeutic formulations, medicaments and prophylactic formulations of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as the compounds, products or expanded NK cell populations of the invention) are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine.

Generally, the carrier is a pharmaceutically-acceptable carrier. Non-limiting examples of pharmaceutically acceptable carriers include water, saline, and phosphate-buffered saline. In some embodiments, however, where the composition comprises a compound or products of the invention, this may be in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, AS01, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

The dosage ranges for administration of the compounds or products of the present invention are those which produce the desired therapeutic effect. It will be appreciated that the dosage range required depends on the precise nature of the compound or products, the route of administration, the nature of the formulation, the age of the patient, the nature, extent or severity of the patient's condition, contraindications, if any, and the judgement of the attending physician. Variations in these dosage levels can be adjusted using standard empirical routines for optimisation. Similarly, the dose of a compound or products of the invention for use in a method of the invention, particularly an ex vivo method, can be readily determined by one of skill in the art, and is any dose that produces the desired increase in NK cell number and/or elicits the desired expansion in NK cells, to produce an expanded NK cell population. As a non-limiting example, doses of SR8278 according to the present invention may give rise to a final concentration of about 2 to about 20 μM, about 2 to about 15 μM, about 5 to about 15 μM, about 5 to about 14 μM, about 4 to about 13 μM, about 5 to about 12 μM, about 5 to about 11 μM, or preferably about 5 to about 10 μM.

The invention also provides the use of an expanded NK cell population (as described herein) in a pharmaceutical formulation. Any and all of the disclosure herein in relation to formulations of a compound of the invention may apply equally and independently to therapeutic applications of the expanded NK cell populations of the invention.

```
Key to SEQ ID NOs
Delta-like ligand 4 gene sequence (AF253468.1)
SEQ ID NO: 1
      1 atggcggcag cgtcccggag cqcctctggc tgggcgctac tgctgctggt gqcactttgg
    61 cagcagcgcg cggccggctc cggcgtcttc cagctgcagc tgcaggagtt catcaacgag
  121 cgcggcgtac tggccagtgg gcggccttgc gagcccggct gccggacttt cttccgcgtc
  181 tgccttaagc acttccaggc ggtcgtctcg cccggaccct gcaccttcgg gaccgtctcc
  241 acgccggtat tgggcaccaa ctccttcgct gtccgggacg acagtagcgg cggggggcgc
  301 aacectctcc aactgccctt caatttcacc tggccgggta ccttctcgct catcatcgaa
  361 gcttggcacg cgccaggaga cgacctgcgg ccagaggcct tgccaccaga tgcactcatc
  421 agcaagatcg ccatccaggg ctccctagct gtgggtcaga acrggttatt ggatgageaa
  481 accagcaccc tcacaaggcc gcgctactct taccgggtca tctgcagtga caactactat
  541 ggagacaact gctcccgcct gtgcaagaag cgcaatgacc acttcggcca ctatgtgtgc
  601 cagccagatg gcaacttgtc ctgcctgccc ggttggactg gggaatattg ccaacagcct
  661 atctgrcttt cgggctgtca tgaacagaat ggctactgca gcaagccagc agagtgcctc
  721 tgccgcccag gccgqcaggg ccggctgtgt aacgaatgca tcccccacaa tggctgtcgc
  781 cacggcacct gcagcactcc ctggcaatgt acttgtgatg agggctgggg aggcctgttt
  841 tgtgaccaag atctcaacta ctgcacccac cactccccat gcaagaatgg ggcaacgtgc
  901 tccaacagtg ggcagcgaag ctacacctgc acctgtcgcc caggctacac tggtgtggac
  961 tgtgagctgg agctcagcga gtgtgacagc aacccctgtc gcaatggagg cagctgtaag
1021 gaccaggagg atggctacca ctgcctgtgt cctccgggct actatggcct gcattgtgaa
1081 cacagcacct tgagctgcgc cgactccccc tgcttcaatg ggggctcctg ccgggagcgc
1141 aaccaggggg ccaactatgc ttgtgaacgt ccccccaact tcaccggctc caactgcgag
1201 aagaaagtgg acaggtgcac cagcaacccc tgtgccaacg ggggacagtg cctgaaccga
1261 ggtccaagcc gcatgtgccg ctgccgtcct ggattcacgg gcacctactg tgaactccac
1321 gtcagcgact gtgcccgtaa cccttgcgcc cacggtggca ctcgccatga cctggagaat
1381 gggctcatgt gcacctgccc tgccggcttc tctggccgac gctgtgaggt gcggacatcc
1441 atcga&gcct gtgcctcgag tccctgcttc aacagggcca cctgctacac cgacctctcc
1501 acagacacct ttgtgtgcaa ctgcccttat ggctttgtgg gcagccgctg cgagttcccc
1561 gtgggcttgc cgcccagctt cccctgggtg gccgtctcgc tgggtgtggg gctggcagtg
1621 ctgctggtac tgctgggcat ggtggcagtg gctgtgcggc agccgcggct tcgacggccg
1681 gacgacggca gcagggaagc catgaacaac ttgtcggact tccagaagga caacctgatt
1741 cccgccgccc agcttaaaaa cacaaaccag aagaaggagc tggaagtgga ctgtggcctg
1801 gacaagtcca accgtggcaa acagcaaaac cacacattgg accataatct ggccccaggg
1861 cccctggggc gggggaccat gccaggaaag tttccccaca gtgacaagag cttaggagag
1921 aaggcgccac tgcggttaca cagtgaaaag ccagagtgtc ggatatcagc gatatgctcc
1981 cccagggact ccacgtacca gtctgtgrgt ttgatatcag aggagaggaa tgaatgtgtc
2041 attgccacgg aggtataa
Delta-like ligand 4 amino acid sequence (AF253468.1)
SEQ ID NO: 2
MAAASRSASGWALLLLVALWQQRAAGSGVFQLQLQEFINERGVLASGRPCEPGCRTFFRVCLKKFQAVVSPGPCT
FGTVSTPVLGTNSFAVRDDSSGGGRNPLQLPFNFTWPGTFSLIIEAWHAPGDDLRPEALPPDALISKIAIQGSLA
VGQNWLLDEQTSTLTRLRYSYRVICSDNYYGDNCSRLCKKRNDHFGHYVCQPDGNLSCLPGWTGEYCQQPICLSG
CHEQNGYCSKPASCLCRPGWQGRLCNECIPHMGCRHGTCSTPWQCTCDEGWGGLFCDQDLNYCTHHSPCKNGATC
SNSGQRSYTCTGRPGYTGVDCELELSECDSNPCRNGGSCKDQEDGYHCLCPPGYYGLHCEHSTLSCADSPCFNGG
SCRERNQGANYACECPPNFTGSNCEKKVDRCTSNPCANGGQCLNRGPSRMCRCRPGFTGTYCELHVSDCARNPCA
HGGTCHDLENGLMCTCPAGFSGRRCEVRTSIDACASSPCFNRATCYTDLSTDTFVCNCPYGFVGSRCEFPVGLPP
SFPWVAVSLGVGLAVLLVLLGMVAVAVRQLRLRRPDDGSREAIWNLSDFQKDNLIPAAQLKNTNQKKELEVDCGL
DKSKCGKQQNHTLDYNLAPGPLGRGTMPGKFPHSDKSLGEKAPLRLHSEKPECRISAICSPRDSMYQSVCLISEE
RNECVIATEV
E4bp4 gene sequence (X64318.1)
SEQ ID NO: 3
      1 gcccctttct ttcrcctcgt cggcccgaga gcaggaacac gataacgaag gaggcccaac
    61 ttcacccaat aaggagcctg acggatttat cccagacggt agaacaaaag gaagaatatt
  121 gatggatttt aaaccagagt ttttaaagag cttgagaata cggggaaatt aatttgttct
  181 cctacacaca tagatagggt aaggttgttt ctgatgcagc tgagaaaaat gcagaccgtc
  241 aaaaaggagc aggcgtctct tgatgccagt agcaatgtgg acaagatgat ggtccttaat
  301 tctgctttaa cggaagtgtc agaagactcc acaacaggtg aggacgtgct tctcagtgaa
  361 ggaagtgtgg ggaagaacaa atcttctgca tgtcggagga aacgggaatt cattcctgat
  421 gaaaagaaag atgctatgta ttgggaaaaa aggcggaaaa ataatgaagc tgccaaaaga
  481 tctcgtgaga agcgtcgact gaatgacctg gttttagaga acaaactaat tgcactggga
  541 gaagaaaacg ccactttaaa agctgagctg ctttcactaa aattaaagtt tggtttaatt
  601 agctccacag catatgctca agagattcag aaactcagta atcctacagc tgtgtacctt
  661 caagattacc agacttccaa atccaatgtg agttcatttg tggacgagca cgaaccctcg
  721 atggtgtcaa gtagttgtat ttctgtcatt aaacactctc cacaaagctc gctgtccgat
  781 gtttcagaag tgtcctcagt agaacacacg caggagagct ctgcgcaggg aagctgcaga
  841 agtcctgaaa acaagttcca gattatcaag caagagccga tggaattaga gagctacaca
  901 agggagccaa gagatgaccg aggctctcac acagcgtcca tctatcaaaa ctatatgggg
  961 aattctttct ctgggtactc acactctccc ccactactgc aagtcaaccg atcctccagc
1021 aactccccga gaacgtcgga aactgatgat ggtgtggtag gaaagtcatc tgatggagaa
1081 gacgagcaac aggcccccaa gggccccatc cattctccag ttgaactcaa gcatgtgcat
1141 gcaaccgtgg ttaaagttcc agaagtgaat tcctccgcct tgccacacaa gctccggatc
1201 aaagccaaag ccacgcagat caaagtagaa gcctttgata atgaatttga ggccacgcaa
1261 aaactttcct cacctattga catgacatct aaaagacatt ccgaactcga aaagcatagt
1321 gccccaagta tggtacattc ttctcttact ccttcctcag tgcaagtgac taacattcaa
1381 gattggtctc tcaaatcgga gcactggcat caaaaagaac tgagtggcaa aactcagaat
1441 agtttcaaaa ctggagttgt tgaaatgaaa gacagtggct acaaagtttc tgacccagag
1501 aacttgtatt tgaagcaggg gatagcaaac ttatctgcag aggtttgtcc actcaagaga
1561 cttatagcca cacaaccaat ctctgcttca gactctgggt aaattactac tgagtaagag
1621 ctgggcattt agaaagatgt catttgcaat agagcagtcc attttgtatt atgctgaatt
1681 ttcactggac ctgtgatgtc atttcactgt gatgtgcaca tgttgtctgt ttggtgtctt
1741 tttgtgcaca gattatgatg aagattagat tgtgttatca ctctgcctgt gtatagtcag
1801 atagtcatat gcgtaaggct gtatatatta agnttttatt tttgttgttc tattataaag
1861 tgtgtaagtt accagtttca ataaaggact ggtgacaaac acagaaaaaa aaaaaaaaaa
1921 aaa
E4bp4 amino acid sequence (X64318.1)
SEQ ID NO: 4
MQLRKMQTVKKEQASLDASSNVDKMMVLNSALTEVSEDSTTGEDVLLSEGSVGKNKSSACRRKREFIPDEKKDAM
YWEKRRKNNEAAKRSREKRRLNDLVLENKLIALGEEMATLKAELLSLKLKFGL1SSTAYAQEIQKLSNSTAVYFQ
DYQTSKSNVSSFVDEHEPSMVSSSCISVIKHSPQSSLSDVSEVSSVEHTQESSVQGSCRSPENKFQIIKQEPMEL
ESYTREPRDDRGSYTASIYQNYMGNSFSGYSHSPPLLQVNRSSSNSPRTSETDDGVVGKSSDGEDEQQVPKGPIH
SPVELKHVHATVVKVPEVNSSALPHKLRIKAKAMQIKVEAFDNEFEATQKLSSPIDMTSKRHFELEKHSAPSMVH
SSLTPFSVQVTNIQDWSLKSEHWHQKELSGKTQMSFKTGVVEMKDSGYKVSDPEMLYLKQGIANLSAEVVSLKRL
IATQPISASDSG
REV-ERBα gene sequence (NM_021724.4)
SEQ ID NO: 5
      1 gggcacgagg cgctccctgg gatcacatgg tacccgctcc agtgccgcgt gcggcccggg
    61 aaccctgggc tgctggcgcc tgcgcagagc cctctgtccc agggaaaggc tcgggcaaaa
  121 ggcggctgag attggcagag tgaaatatta ctgccgaggg aacgtagcag ggcacacgtc
  181 tcgcctcttt gcgactcggt gccccgtttc tccccatcac ctacttactt cctggttgca
  241 acctctcttc ctctgggact tttgcaccgg gagctccaga ttcgccaccc cgcagcgctg
  301 cggagccggc aggcagaggc accccgtaca ctgcagagac ccgaccctcc ttgctacctt
  361 ctagccagaa ctactgcagg ctgattcccc ctacacactc tctctgctct tcccatgcaa
  421 agcagaactc cgttgcctca acgtccaacc cttctgcagg gctgcagtcc ggccacccca
  481 agaccttgct gcagggtgct tcggatcctg atcgtgagtc gcggggtcca ctccccgccc
  541 ttagccagtg cccagggggc aacagcggcg atcgcaacct ctagtttgag tcaaggtcca
  601 gtttgaatga ccgctctcag ctggtgaaga catgacgacc ctggactcca acaacaacac
  661 aggtggcgtc atcacctaca ttggctccag tggctcctcc ccaagccgca ccagccctga
  721 atccctctat agtgacaact ccaatggcag cttccagtcc ctgacccaag gctgtcccac
  781 ctacttccca ccatccccca ctggctccct cacccaagac ccggctcgct cctttgggag
  841 cattccaccc agcctgagtg atgacggctc cccttcttcc tcatcttcct cgtcgtcatc
  901 ctcctcctcc ttctataatg ggagcccccc tgggagtcta caagtggcca tggaggacag
  961 cagccgagtg tcccccagca agagcaccag caacatcacc aagctgaatg gcatggtgtt
1021 actgtgtaaa gtgtgtgggg acgttgcccc gggcttccac tacggtgtgc acgcctgcga
1081 gggctgcaag ggctttttcc gtcggagcat ccagcagaac atccagtaca aaaggtgtct
1141 gaagaatgag aattgctcca tcgtccgcat caatcgcaac cgctgccagc aatgtcgctt
1201 caagaagtgt ctctctgtgg gcatgtctcg agacgctgtg cgttttgggc gcatccccaa
1261 acgagagaag cagcggatgc ttgctgagat gcagagtgcc atgaacctgg ccaacaacca
1321 gttgagcagc cagtgcccgc tggagacttc acccacccag caccccaccc caggccccat
1381 gggcccctcg ccaccccctg ctccggtccc ctcacccctg gtgggcttct cccagtttcc
1441 acaacagctg acgcctccca gatccccaag ccctgagccc acagtggagg atgtgatatc
1501 ccaggtggcc cgggcccatc gagagatcct cacctacgcc cacgacaagc tgggcagctc
1561 acctggcaac ttcaatgcca accatgcatc aggtagccct ccagccacca ccccacatcg
1621 ctgggaaaat cagggctgcc cacctgcccc caatgacaac aacaccttgg ctgcccagcg
1681 tcataacgag gccctaaatg gtctgcgcca ggctccctcc tcctaccctc ccacctggcc
1741 tcctggccct gcacaccaca gctgccacca gtccaacagc aacgggcacc gtctatgccc
1801 cacccacgtg tatgcagccc cagaaggcaa ggcacctgcc aacagluccc ggcagggcaa
1861 ctcaaagaat gttctgctgg catgtcctat gaacatgtac ccgcatggac gcagtgggcg
1921 aacggtgcag gagatctggg aggatttctc catgagcttc acgcccgctg tgcgggaggt
1981 ggtagagttt gccaaacaca tcccgggctt ccgtgacctt tctcagcatg accaagtcac
2041 cctgcttaag gctggcacct ttgaggtgct gatggtgcgc tttgcttcgt tgttcaacgt
2101 gaaggaccag acagtgatgt tcctaagccg caccacctac agcctgcagg agcttggtgc
2161 catgggcatg ggagacctgc tcagtgccat gtccgacttc agcgagaagc tcaactccct
2221 ggcgcttacc gaggaggagc tgggcctctt caccgcggtg gtgcttgtct ctgcagaccg
2281 ctcgggcatg gagaattccg cttcggtgga gcagctccag gagacgctgc tgcgggctct
2341 tcgggctctg gtgctgaaga accggccctt ggagacttcc cgcttcacca agctgctgct
2401 caagctgccg gacctgcgga ccctgaacaa catgcattcc gagaagctgc tgtccttccg
2461 ggtggacgcc cagtgacccg cccggccggc cttctgccgc tgcccccttg tacagaatcg
2521 aactctgcac ttctctctcc tttacgagac gaaaaggaaa agcaaaccag aatcttattt
2581 atattgttat aaaatattcc aagatgagcc tctggccccc tgagccttct tgtaaatacc
2641 tgcctccctc ccccatcacc gaacttcccc tcctccccta tttaaaccac tctgtctccc
2701 ccacaaccct cccctggccc tctgatttgt tctgttcctg tctcaaatcc aatagttcac
2761 agctgagctg gcttcaaaaa aaaaaaaaaa aaa
REV-ERBα amino acid sequence (NM_021724.4)
SEQ ID NO: 6
MTTLDSNNNTGGVITYIGSSGSSPSRTSPESLYSDNSNG3FQSLTQGCPTYFPPSPTGSLTQDPARSFGSIPPSL
SDDGSPSSSSSSSSSSSSFYKGSPPGSLQVAMEDSSRVSPSKSTSNITKLNGMVLLCKVCGDVASGFHYGVHACE
GCKGFFRRSIQQNIQYKRCLKNENCSIVRINRNRCQQCRFKKCLSVGMSRDAVRFGRIPKREKQRMLAEMQSAMN
LANKQLSSQCPLETSPTQHPTPGPMGPSPPPAPVPSPLVGFSQFPQQLTPPRSPSPEPTVEDVISQVARAHREIF
TYAHDKLGSSPGNFNANHASGSPPATTPHRWENQGCPPAPNDNNTLAAQRHNEALNGLRQAPSSYPPTWPPGPAH
HSCHQSNSNGKRLCPTHVYAAPEGKAPANSPRQGNSKNVLLACPMNMYPHGRSGRTVQEIWEDFSMSFTPAVREV
VEFAKHIPGFRDLSQHDQVTLLKAGTFEVLMVRFASLFNVKDQTVMFLSRTTYSLQELGAMGMGDLLSAMFDFSE
KLNSLALTEEELGLFTAVVLVSADRSGMENSASVEQLQETLLRALRALVLKNRPLETSRFTKLLLKLPDLRTLNN
MHSEKLLSFRVDAQ
REV-ERBβ gene sequence (A8307693.1)
SEQ ID NO: 7
      1 atggaggtga atgcaggagg ugtgattgcc tatatcagtt cttccagctc agcctcaagc
    61 cctgcctctt gtcacagtga gggttctgaq aatagtttcc agtcctcctc ctcttctgtt
  121 ccatcttctc caaatagctc taattctgat accaatggta atcccaagaa tggtgatctc
  181 gccaatattg aaggcatctt gaagaatgat cgaatagatt gttctatgaa aacaagcaaa
  241 tcgagtgcac ctgggatgac aaaaaatcat agtggtgtga caaaatttag tggcatggtt
  301 ctactgtgta aagtctgtgg ggatgtggcg tcaggattcc actatggagt tcatgcttgc
  361 gaaggctgta agggtttctt tcggagaagt attcaacaaa acatccagta caagaagtgc
  421 ctgaagaatg aaaactgttc tataatgaga atgaatagga acagatgtca gcaatgtcgc
  481 ttcaaaaagt gtctgtctgt uggaatgtca agagacgctg ttcggtttgg Ccgtattcct
  541 aagcgtgaaa aacaqaggat gctaattgaa atgcaaagtg caatgaagac catgatgaac
  601 agccagttca gtggtcactt gcaaaatgac acattagtag aacatcatga acagacagcc
  661 ttgccagccc aggaacagct gcgacccaag ccccaactgg agcaagaaaa catcaaaagc
  721 tcttctcctc catcttctga ttttgcaaag gaagaagtga ttggcatggt gaccagagct
  781 cacaaggata cctttatgta caatcaagag cagcaagaaa actcagctga gagcatgcag
  841 ccccagagag gagaacggat tcccaagaac atggagcaat ataatttaaa tcatgatcat
  901 tgcggcaatg ggcttagcag ccattttccc tgtagtgaga gccagcagca tctcaatgga
  961 cagttcaaag ggaggaatat aatgcattac ccanatggcc atgccatttg tattgcaaat
1021 ggacattgta tgaacttctc caatgctcat actcaaagag tatgtgatag agttccgata
1081 gatggatttt ctcagaatga gaacaagaat agttacctgt gcaacactgg aggaagaatg
1141 catctggttt gtccaatgag taagtctcca tatgtggatc ctcataaatc aggacatgaa
1201 atctgggaag aattttcgat gagcttcact ccagcagtga aagaagtggt ggaatttgca
1261 aagcgtattc ctgggttcag agatctctct cagcatgacc aggtcaacct tttaaaggct
1321 gggacttttg aggttttaat ggtacggttc gcatcattat ttgatgcaaa ggaacgtact
1381 gtcacctttt taagtggaaa gaaatatagt gtggatgatt tacactcaat gggagcaggg
1441 gatctgctaa actctatgtt tgaatttagt gagaagctaa atgccctcca acttagtgat
1501 gaagagatga gtttgtttac agctgttgtc ctggtatctg cagatcgatc tggaatagaa
1561 aacgtcaact ctgtggaggc tttgcaggaa actctcattc gtgcactaag gaccttaata
1621 atgaaaaacc atccaaatga ggcctctatt tttacaaaac tgcttctaaa gttgccagat
1681 cttcgatctt taaacaacat gcactctgag gagctcttgg cctttaaagt tcacccttaa
REV-ERBβ amino add sequence (AB307693.1)
SEQ ID NO: 8
MEVNAGGVIAYISSSSSASSPASCHSEGSENSFQSSSSSVPSSPNSSNSDTNGNPKNGDLRNIEGILKKDRIDCS
MKTSKSSAPGMTKNHSGVTKFSGMVLLCKVCGDVASGFHYGVHACEGCKGFFRRSIQQNIQYKKCLKNENCSIMR
MNRNRCQQCRFKKCLSVGMSRDAVRFGRIPKREKQRMLIEMQSAMKTMMNSQFSGHLQNDTLVEHHEQTALPAQE
QLRPKPQLEQENIKSSSPPSSDFAKEEVIGMVTRAHKDTFMYNQEQQENSAESMQPQRGERIPKNMEQYNLNHDH
CGNGLSSHFPCSESQQHLNGQFKGRNIMHYPXGHAICIANGHCMNFSNAYTQRVCDRVPIDGFSQNENKNSYLCN
TGGRMHLVCPMSKSPYVDPHKSGHEIWEEFSMSFTPAVKEVVEFAKRIPGFRDLSQHDQVNLLKAGTFEVLMVRF
ASLFDAKERTVTFLSGKKYSVDDLHSMGAGDLLMSMFSFSEKLNALQLSDEEMSLFTAVVLVSADRSGIENVNSV
EALQETLIRALRTLIMKMHPNHASIFTKLLLKLPDLRSLNNMHSEELLAFKVH
forward primer A for detection of E4bp4 wildtype allele
SEQ ID NO: 9
CTCTGAGCTTGGCTGATGTG
reverse primer for the detection of E4bp4
SEQ ID NO: 10
GCTTCAAGTCTCCACCAAGC
primer for the detection of the E4bp4 null allele
SEQ ID NO: 11
CCATGCTCCTGTCTTGATGA
side chain on SUMO modified peptide
SEQ ID NO: 12
GGTQQQFV
mouse E4bp4 peptide comprising potential SUMO modification site
SEQ ID NO: 13
MQLRKMQTKKEPAPLDPTS
rat E4bp4 peptide comprising potential SUMO modification site
SEQ ID NO: 14
MQLRKMQAIKKEPASLDPTG
SEQ ID NO: 15
human E4bp4 peptide comprising potential SUMO modification site
MQLRK M QTVKKEQASLDASS
chicken E4bp4 peptide comprising potential SUMO modification site
SEQ ID NO: 16
MQLRKMQTLKKEHGSVDTSS
Xenopus E4bp4 peptide comprising potential SUMO modification site
SEQ ID NO: 17
MPTIKKEQECADSRM
mouse E4bp4 peptide comprising potential SUMO modification site
SEQ ID NO: 18
LENKLIALGEENATLKAELL
rat E4bp4 peptide comprising potential SUMO modification site
SEQ ID NO: 19
LENKLIALGEENATLKAELL
human E4bp4 peptide comprising potential SUMO modification site
SEQ ID NO: 20
LENKLIALGEENATLKAELL
chicken E4bp4 peptide comprising potential SUMO modification site
SEQ ID NO: 21
LENKLIALGEENATLKAELL
Xenopus E4bp4 peptide comprising potential SUMO modification site
SEQ ID NO: 22
LENKLIALGEENASLKTELL
mouse E4bp4 peptide comprising potential SUMO modification site
SEQ ID NO: 23
PENKFPVIKQEPVELESFAR
rat E4bp4 peptide comprising potential SUMO modification site
SEQ ID NO: 24
PENKFPVIKQEPVELESFAR
human E4bp4 peptide comprising potential SUMO modification site
SEQ ID NO: 25
PENKFQIIKQEPMELESYTR
chicken E4bp4 peptide comprising potential SUMO modification site
PENKFQIIKQEPIELER
SEQ ID NO: 26
Xenopus E4bp4 peptide comprising potential SUMO modification site
SEQ ID NO: 27
TDIKSQRIKQEQMEAGNFSR
mouse E4bp4 peptide comprising potential SUMO modification site
SEQ ID NO: 28
RIKAKAMQVKVEALDSEFEG
rat E4bp4 peptide comprising potential SUMO modification site
SEQ ID NO: 29
RIKAKAMQVKVEALDSEFEG
human E4bp4 peptide comprising potential SUMO modification site
SEQ ID NO: 30
RIKAKAMQIKVEAFDNEFEA
chicken E4bp4 peptide comprising potential SUMO modification site
SEQ ID NO: 31
RIKAKAMQVKVEAM DNDYDA
Xenopus E4bp4 peptide comprising potential SUMO modification site
SEQ ID NO: 32
RIKAKAMQIKVESLESELNS
mouse E4bp4 peptide comprising potential SUMO modification site
SEQ ID NO: 33
VTNIQDWSLKSEHWHHKELS
rat E4bp4 peptide comprising potential SUMO modification site
SEQ ID NO: 34
VTN iQDWSLRSE HWHH KELG
human E4bp4 peptide comprising potential SUMO modification site
SEQ ID NO: 35
VTNIQDWSLKSEHWHQKELS
chicken E4bp4 peptide comprising potential SUMO modification site
SEQ ID NO: 36
VTNIQDWSLKPELWHQKELN
Xenopus E4bp4 peptide comprising potential SUMO modification site
SEQ ID NO: 37
VTNIQDWPLKPGQWHHRELE
Forward primer for amplification of putative E4bp4
binding regions in Notchl locus
SEQ D NO: 38
CTATATTTTTGCCTTGACAGCTAAAGG
Reverse primer for amplification of putative E4bp4
SEQ ID NO: 39
binding regions in Notchl locus
GAAGTACGAAGCATGCTTGC
Forward primer for amplification of putative E4bp4
binding regions in Notchl locus
SEQ ID NO: 40
CACATCTGTGAGCTATTTTTGG
Reverse primer for amplification of putative E4bp4
binding regions in Notchl locus
SEQ ID NO: 41
GACTGACTAAACTAACATTCCCAC
Forward primer for amplification of putative E4bp4
binding regions in Notchl locus
SEQ ID NO: 42
CTCAGAAACTGGCCTCAAGC
Reverse primer for amplification of putative E4bp4
binding regions in Notchl locus
SEQ ID NO: 43
CACTTGCAGTCAGGCGTTC
Forward primer for amplification of putative E4bp4
binding regions in Notchl locus
SEQ ID NO: 44
CACGCCATCTTAAAGAGCTC
Reverse primer for amplification of putative E4bp4
binding regions in Notchl locus
SEQ ID NO: 45
GTAACCAACTGCACTCTTCTCC
Forward primer for amplification of putative E4bp4
binding regions in Notchl locus
SEQ ID NO: 46
CACCAAGAATTCCCAGGAG
Reverse primer for amplification of putative E4bp4
binding regions in Notchl locus
SEQ ID NO: 47
GAGTGCAGTCACGTGCTGAC
Forward primer for amplification of putative E4bp4
binding regions in Notchl locus
SEQ ID NO: 48
CTCAGACTCTCTCGGTAAGTGTC
Reverse primer for amplification of putative E4bp4
binding regions in Notchl locus
SEQ ID NO: 49
CGTGTGGAGCTACTCTGGC
Human Notchl cDNA sequence (CR457221.1)
SEQ ID NO: 50
      1 atgtcaaaca tgagatgtgt ggactgtggc acttgcctgg gtcacacacg gaggcatcct
    61 accctcttct ggggaaagac actgcctggg ctgaccccgg tggcggcccc agcacctcag
  121 cctgcacagt gtcccccagg ttccgaagaa gatgctccag caacacagcc tgggccccag
  181 ctcgcgggac ccgacccccc gtgggctccc gtgttttgta ggagacttgc cagagccggg
  241 cacattgagc tgtgcaacgc cgtgggctgc gtcctttggt cctgtccccg cagccctggc
  301 agggggcatg cggtcgggca ggggctggag ggaggcgggg gctgcccttg ggccacccct
  361 cctagtttgg gaggagcaga tttttgcaat accaagtata gcctatggca gaaaaaatgt
  421 ctttaa
Human Notch1 protein sequence (CR457221.1)
SEQ ID NO: 51
MSNMRCVDCGTCLGHTRRHPTLFWGKTLPGLTPVAAPAPQPAQCPPGSEEDAPATQPGPQLAGPDPPWAPV
FCRRLARAGHTELCNAVGCVLWSCPRSPGRGHAVGQGLEGGGGCPWATPPSLGGADFCNTKYSLWQKKCL
```

### EXAMPLES

The invention will be further clarified by the following examples, which are intended to be purely exemplary of the invention and are in no way limiting.

### Example 1—E4bp4 is SUMOlylated

To investigate how E4bp4 protein functions might be regulated, a yeast-two-hybrid screen was performed to try to identify binding partners for the E4bp4 protein. Eleven proteins received multiple hits in the screen, but the protein with the highest number of positive identifications was PIAS1 (Table 1). PIAS1 is a small ubiquitin-like modifier (SUMO) E3 ligase required for the addition of post-translational SUMO modifications, suggesting that E4bp4 may be post-translationally SUMOylated.

TABLE 1
E4bp4 interacting partners from yeast-two-hybrid screen.

| Gene Name | Positive clones | Protein function |
|---|---|---|
| C18orf25 | 1 | Unknown |
| CHD1 | 1 | DNA helicase binding protein |
| DSP | 2 | Cytoskeletal protein |
| FLJ13057 | 5 | Unknown |
| FLJ544447 | 1 | Unknown |
| FLNA | 1 | Cytoskeletal protein |
| HIPK1 | 1 | Serine/threonine protein kinase |
| HIPK3 | 5 | Serine/threonine protein kinase |
| PIAS1 | 20 | SUMO E3 ligase |
| PIAS3 | 2 | SUMO E3 ligase |
| RANBP2 | 1 | SUMO E3 iigase |
| RNF111 | 1 | SUMO-targeted ubiquitin ligase |
| SETX | 2 | DNA helicase |
| SNRP70 | 2 | Splicesomal ribonucleoprotein |
| SORL1 | 1 | Neuronal apolipoprotein E receptor |
| TLK2 | 2 | Tousled-like serine/threonine kinase |
| U5-200KD | 1 | RNA helicase |
| ZBTB16 | 1 | Zinc finger transcription factor |
| ZMYM5 | 5 | Zinc Finger MYM-Type Protein |
| ZNF198 | 5 | Zinc Finger Transcriptional cofactor |
| ZNF237 | 5 | Zinc Finger Transcriptional cofactor |

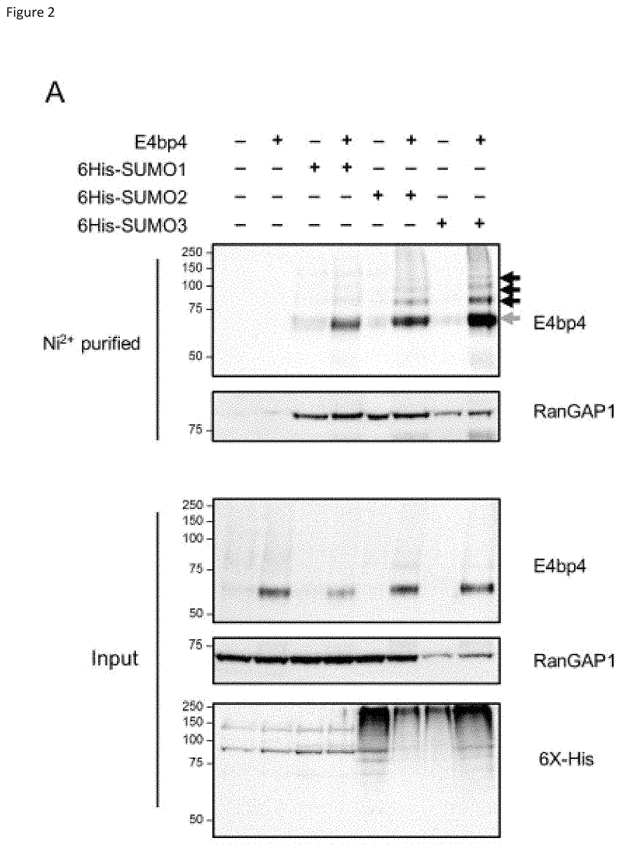

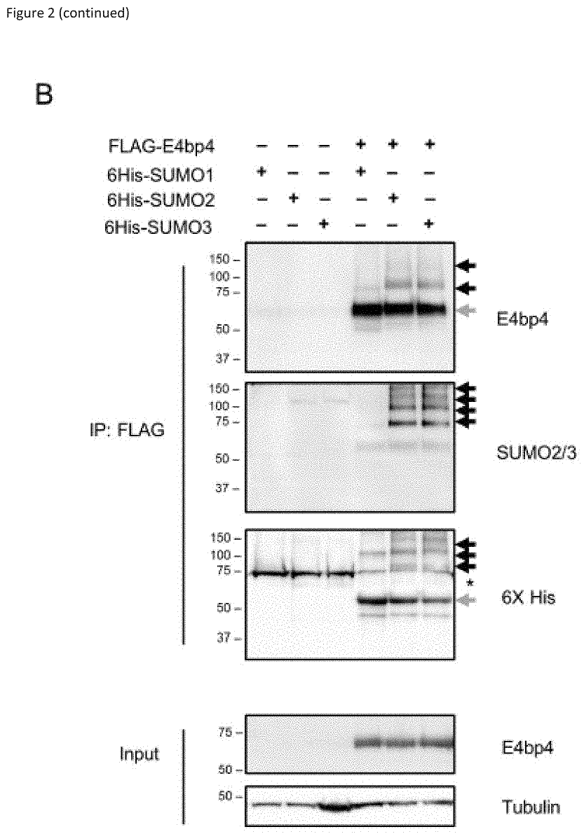

SUMO proteins are reversible post-translational protein modifiers and mammals express four SUMO isoforms, designated SUMO1 to SUMO4. Mature SUMO2 and SUMO3 proteins differ by only three amino acids and are functionally homologous, whilst SUMO4 cannot be efficiently processed in mammalian cells and is not thought to be functional. E4bp4 was expressed in HeLa cells stably expressing 6His-SUMO1, 6His-SUMO2 and 6His-SUMO3 and, following enrichment of all SUMOylated proteins by Ni²⁺ affinity chromatography, higher molecular weight forms of E4bp4 were observed (FIG. 2A). Each SUMO conjugate adds 10-15 kDa onto the apparent molecular weight of a protein, so the higher molecular weight forms of E4bp4 correspond to multiply SUMOylated versions of the protein. FLAG tagged E4bp4 was also expressed in each 6His-SUMO HeLa cell line and purified using anti-FLAG affinity resin and the same multiple higher molecular weight forms of E4bp4 were observed in the presence of 6His-SUMO2 and 6His-SUMO3 (FIG. 2B).

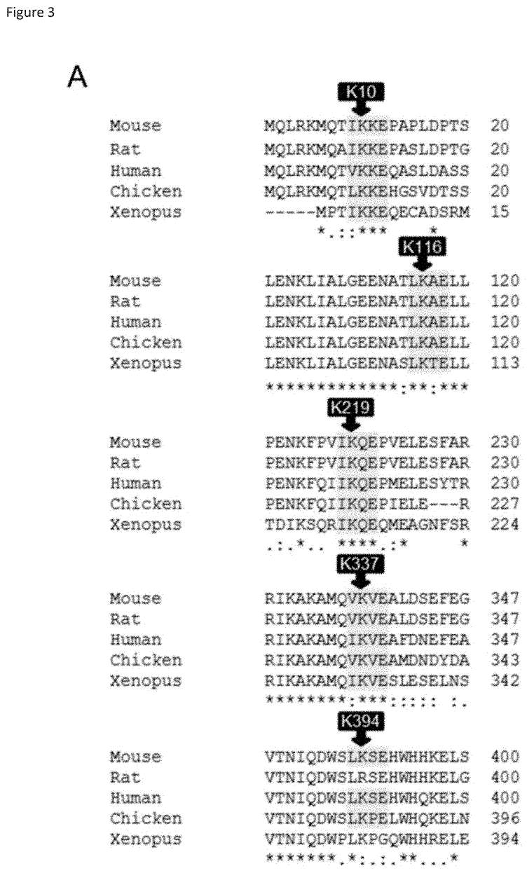

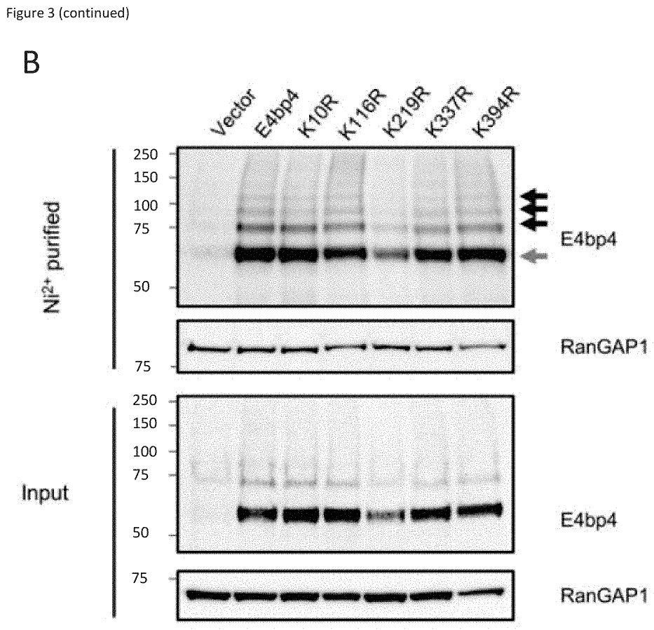

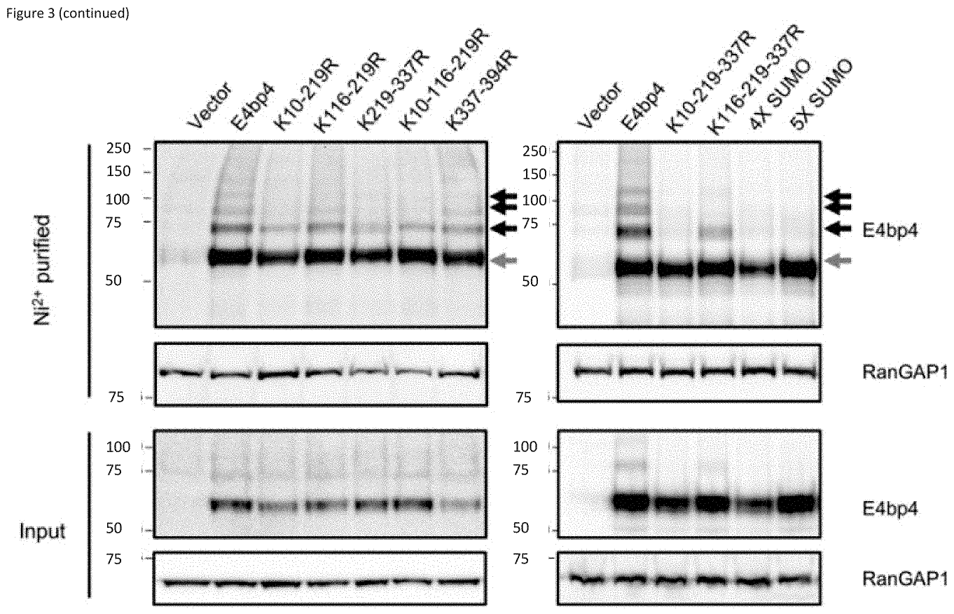

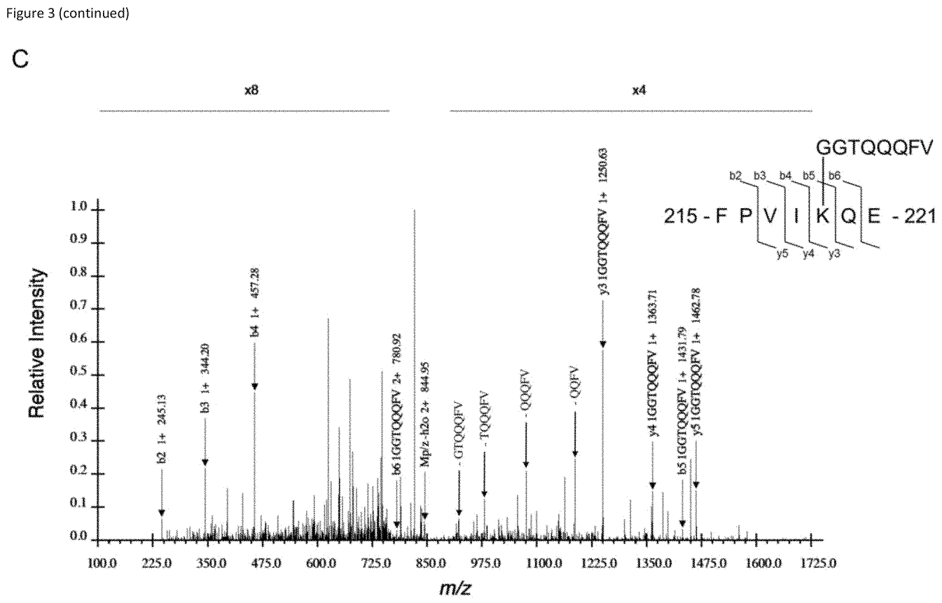

E4bp4 was examined for the presence of the SUMOylation consensus motif ψ-K-x-E in its primary amino acid sequence (FIG. 3A). Five potential modification sites were discovered that were all highly conserved across a range of species (FIG. 3A). To establish if any of these sites were SUMOylated, the central lysine residue at each site was individually mutated to an arginine in the E4bp4 cDNA by site-directed mutagenesis. This mutation abolishes any SUMO modification at that site while maintaining the structural integrity of the protein. To assess the effect of the mutations, each SUMO mutant was expressed in the 6His-SUMO2 HeLa cells (FIG. 3B). E4bp4-K219R was the only individual mutant to affect the SUMOylation of E4bp4, but it did not fully remove all E4bp4 SUMOylation (FIG. 3B). All multi-site mutants affected SUMOylation of E4bp4, in particular, the 5×-SUMO mutant lacking all 5 putative SUMOylation sites, had no higher molecular weight forms of E4bp4 (FIG. 3B).

To confirm the presence of SUMO modifications, purified E4bp4 protein was analysed by mass spectrometry (MS). Studying SUMO modifications by MS is challenging as SUMOylated forms of a protein are generally low in abundance and standard trypsin cleavage results in long SUMO peptide ‘tails’ remaining conjugated to target peptides, making them difficult to detect in standard MS21. A system was developed where FLAG epitope-tagged E4bp4 protein was expressed in 293T cells, purified by immunoprecipitation and sequentially digested with both trypsin and Glu-C. This novel double digest strategy aimed to produce short E4bp4 peptides with reduced SUMO isopeptide side chains on modified peptides. Using this approach a SUMO modified peptide was predicted to have a -GGTQQQFV side chain attached to a modified lysine. MS/MS analysis readily identified an E4bp4 peptide with a SUMO modification at K219, further confirming the presence of this POST-TRANSLATIONAL MODIFICATION (PTM) (FIG. 3C). These data demonstrated that the E4bp4 protein has SUMO modifications.

### Example 2—SUMOylation of E4bp4 Influences NK Cell Development

The potential effect of these SUMO modifications on E4bp4's function as a transcription factor in NK cell development was then investigated.

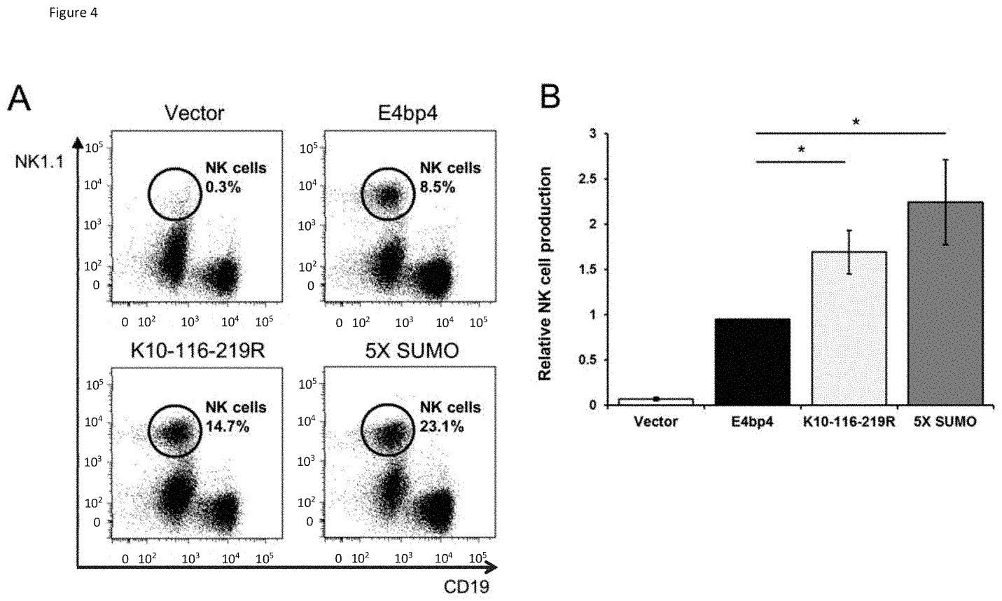

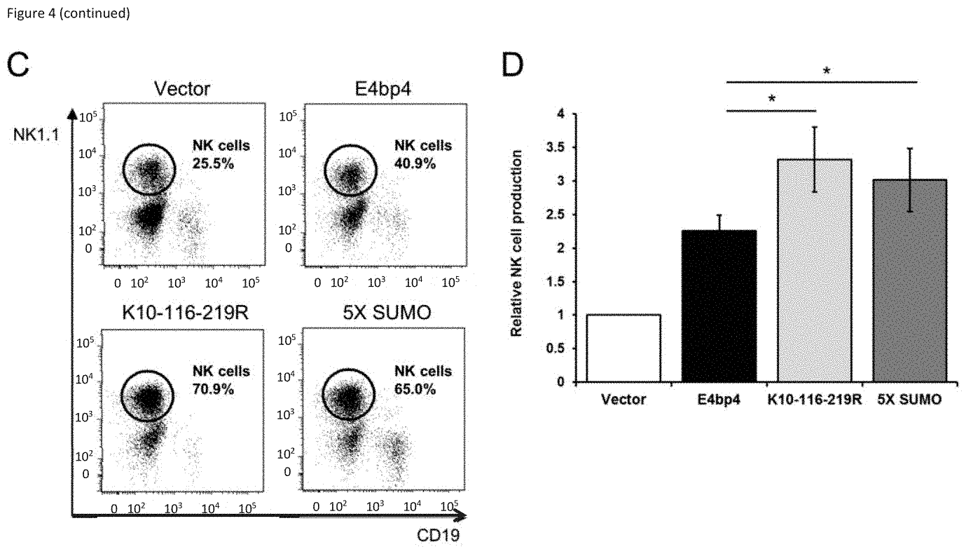

SUMOylation can affect the function of a transcription factor in different ways: cellular localisation; interactions with other proteins or; ability to regulate the expression of target genes. As E4bp4 is critical for the development of NK cells, it was postulated that SUMOylation may regulate this function. Therefore, the ability of E4bp4 to promote NK cell development with and without SUMO modifications was compared. Lineage negative (Lin⁻) BM cells were isolated from E4bp4^{-/-} mice and transduced with a retrovirus expressing either the WT-form of E4bp4 or one of the E4bp4 mutants lacking SUMO modification sites. WT-form E4bp4 rescued NK cell development from E4bp4^{-/-} Lin⁻ BM cells as previously reported, however, the number of NK cells produced was significantly higher when the cells expressed E4bp4 SUMOylation mutants (FIG. 4A-C). As the number of NK cells produced can vary between assays, the percentage of NK cells produced by each mutant was normalised to the positive control condition (i.e. WT-form E4bp4) (FIG. 4B). The same assay was also performed using E4bp4^{+/+} Lin BM cells. In the E4bp4^{+/+} Lin⁻ BM cells, expression of WT-form E4bp4 increased the level of NK cell production compared to the empty vector, but again the expression of E4bp4 SUMO mutants led to significantly greater levels of NK cell production (FIG. 4C,D). These findings showed that SUMOylation can influence the function of E4bp4 and in its absence the activity of E4bp4, as measured by NK cell output, is substantially increased.

### Example 3—E4bp4 is Multiply Phosphorylated and these Modifications Influence NK Cell Development

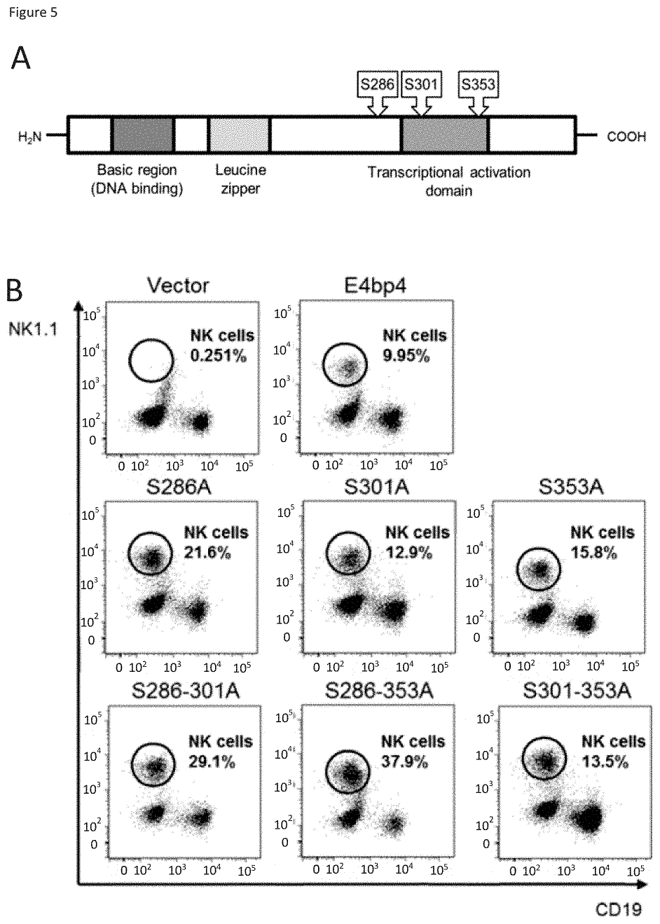

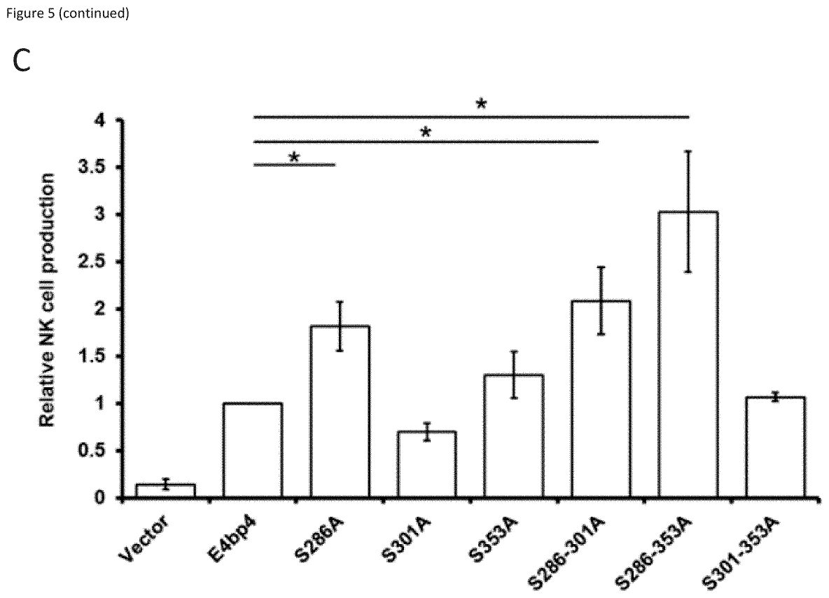

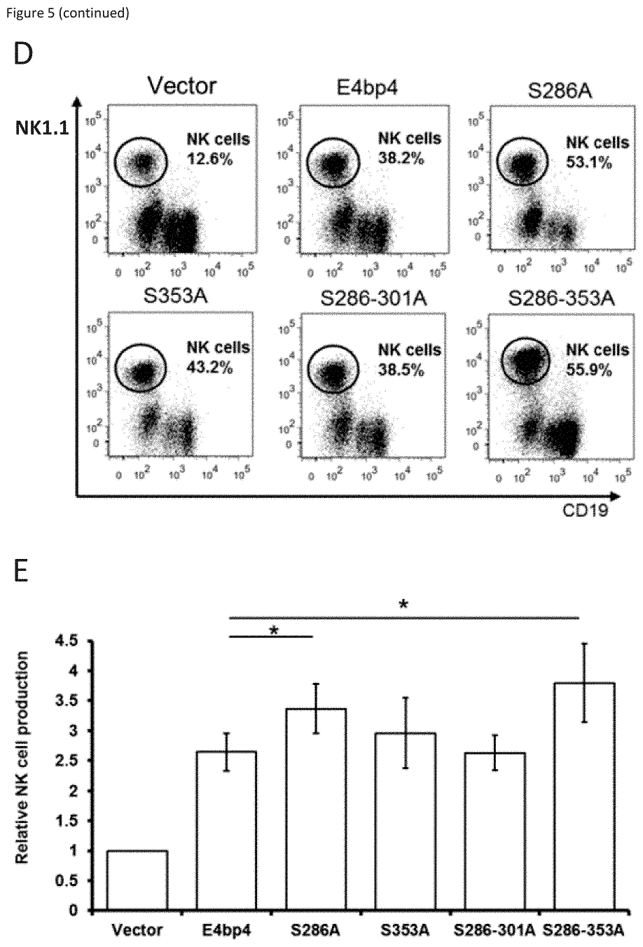

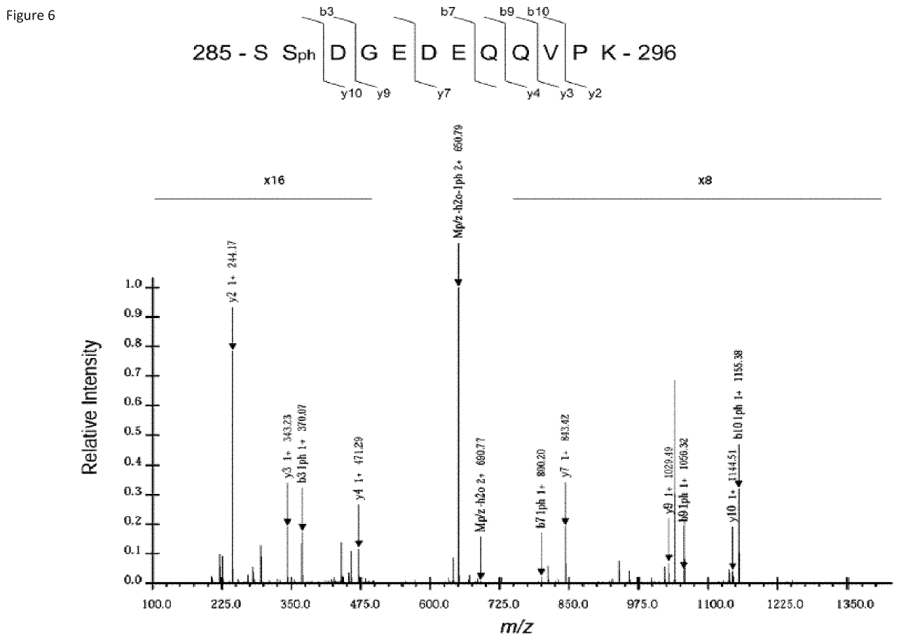

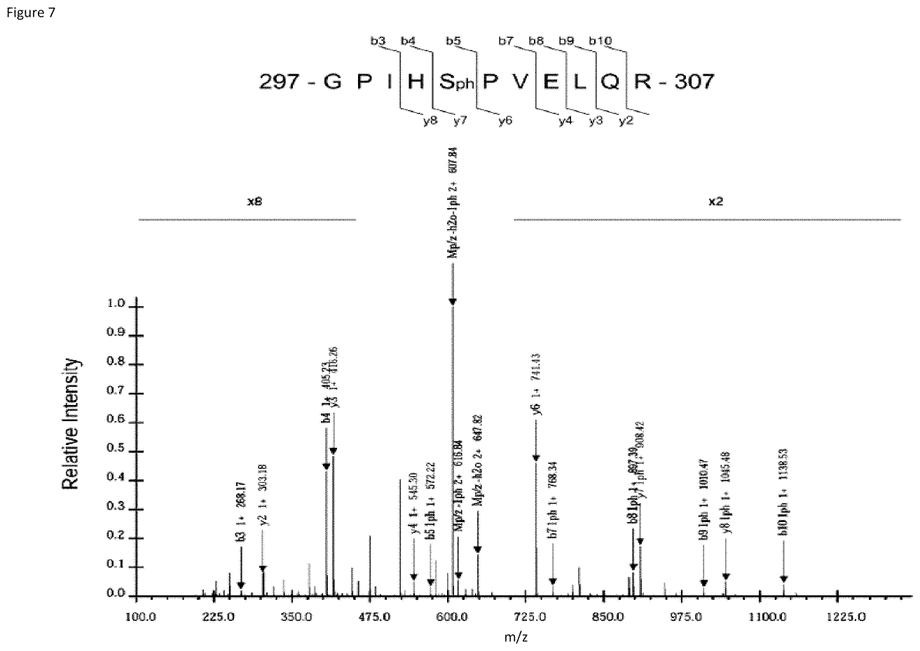

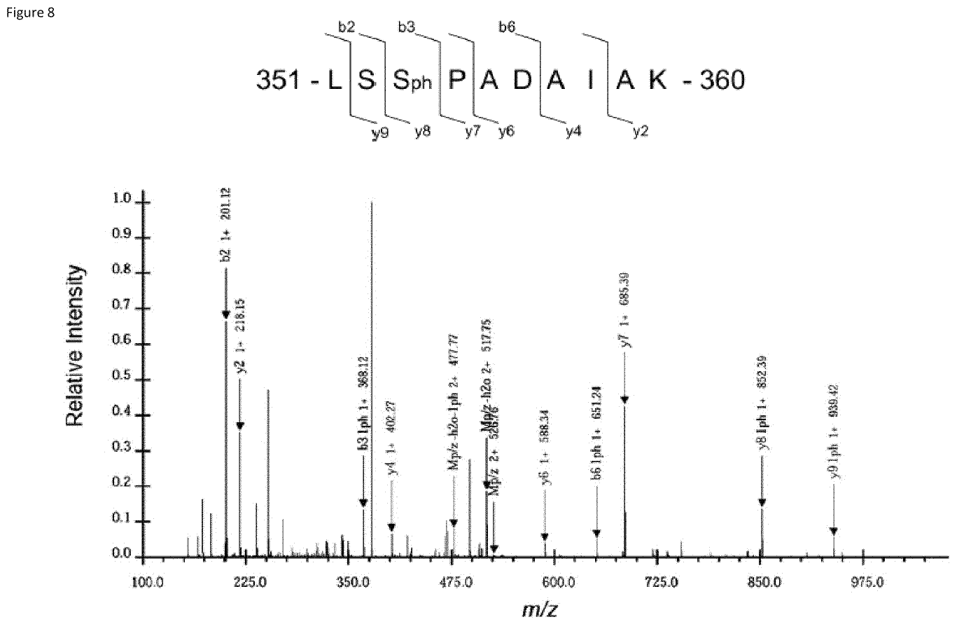

To determine which residues of E4bp4 are phosphorylated FLAG-E4bp4 expressed in 293T cells was purified and digested the protein using trypsin for LC-MS/MS analysis. The tandem MS conclusively revealed that E4bp4 has three phosphorylation sites at serines 286, 301 and 353 (FIGS. 5A and 6-8). Each of these serine residues was mutated to alanine to abolish any phosphorylation but maintain protein conformation. As described for SUMOylation, the aim was to determine if phosphorylation regulates the function of E4bp4. Each phosphorylation mutant was expressed by retroviral expression vector in E4bp4^{-/-} Lin⁻ BM cells and these were cultured in conditions promoting NK cell development. The ability of the phosphorylation mutants to rescue NK cell development was compared to the WT-form of E4bp4 and several of the mutants were found to promote significantly higher levels of NK cell production (FIG. 5B, C). In particular, the S286A mutant produced double the number of NK cells as WT-form E4bp4 (FIG. 5B). The phosphorylation mutants were also transduced into E4bp4^{+/+} Lin⁻ BM cells and NK cell production was greatly enhanced in the cells transduced with the phosphorylation mutants compared to those transduced with the WT-form of E4bp4, particularly with the S286A and S286-353A mutants (FIG. 5E, E). The phenotype observed when phosphorylation sites were mutated replicated that described above for SUMOylation. Therefore, both post-translational modifications (PTMs) negatively regulate the function of E4bp4 in NK cells and manipulating the PTMs of E4bp4 provides a simple mechanism to control NK cell production.

### Example 4—SUMOylation and Phosphorylation do not Affect the Stability of E4bp4

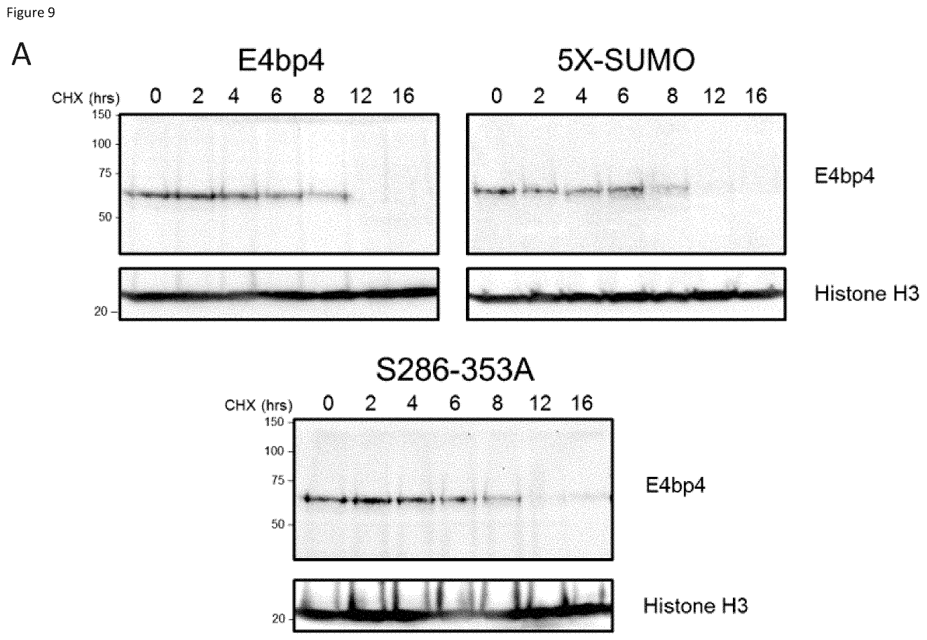

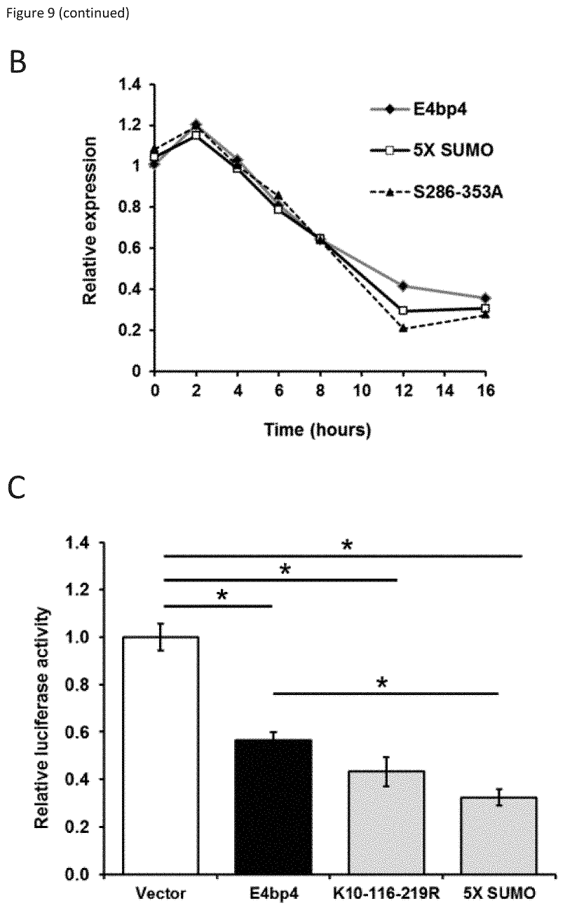

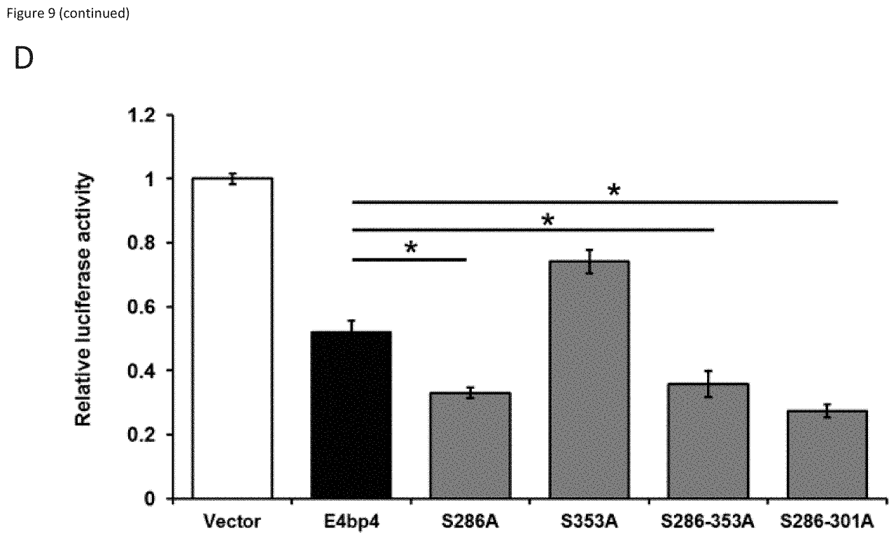

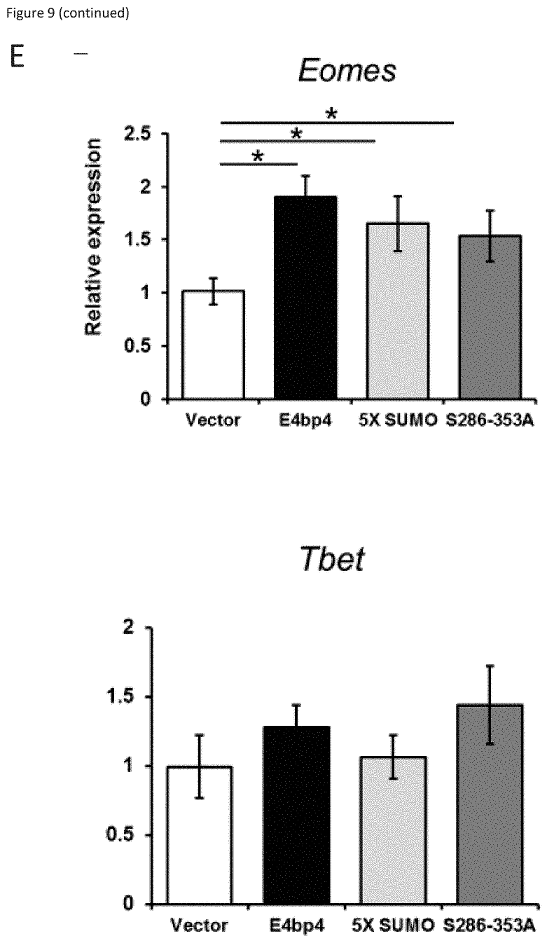

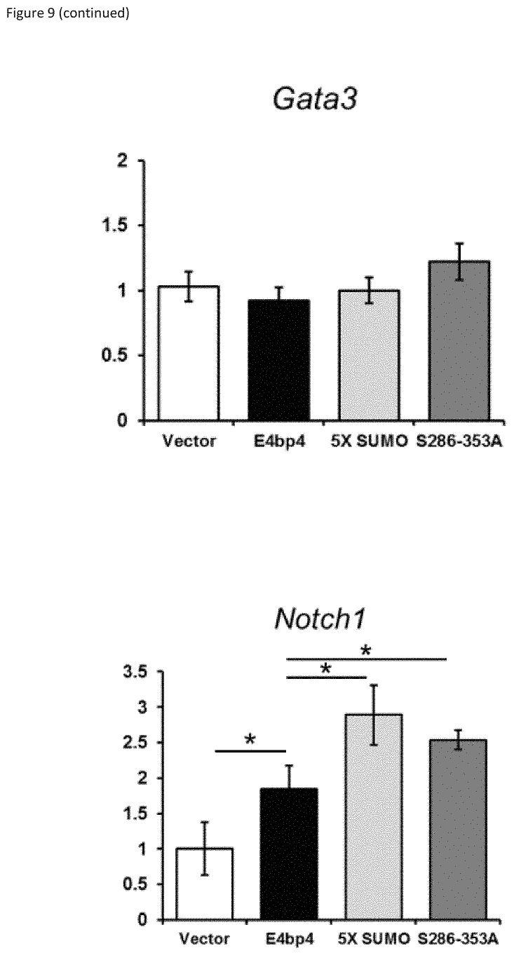

SUMOylation and phosphorylation can both affect the stability of a transcription factor and influence its proteasomal degradation. As the absence of both of these PTMs enhanced the function of E4bp4 during NK cell development, it was investigated whether the mutant versions of E4bp4, lacking PTM sites, had altered stability. Using a cycloheximide time course assay on cell lines stably expressing the 5×-SUMO, S286-353A mutants or the WT-form of E4bp4, the stability of each form of E4bp4 was compared. Both mutants had very similar half-lives to WT E4bp4 with protein levels reduced by almost half after 8 hours of cycloheximide treatment (FIG. 9A, B).

### Example 5—the Transcriptional Activity of E4bp4 can be Regulated by SUMOylation and Phosphorylation

E4bp4 was first identified as a transcriptional repressor and has been shown to repress the expression of numerous target genes in vivo e.g. Il-13 in TH2 cells. However, E4bp4 has likewise been found to transactivate the expression of various target genes, including Id2 and Eomes in NK cells. It was postulated that SUMOylation and phosphorylation might influence the ability of E4bp4 to control target gene expression. To analyse the effect on gene transcription, a luciferase reporter gene assay was used as readout. Cells were co-transfected with an E4bp4 expression vector and a plasmid with three E4bp4 DNA binding sequences upstream of the pGL3 promoter luciferase reporter. E4bp4 was found to act as a transcriptional repressor in this context (FIG. 9C). The WT-form of E4bp4 consistently led to a 50% decrease in luciferase expression, as previously reported 24, but the E4bp4 5×-SUMO mutant promoted even greater transcriptional repression than the WT-form of E4bp4 (FIG. 9C). A similar observation was observed for the E4bp4 phosphorylation mutants, particularly those containing a S286A mutant residue (FIG. 9D). These data indicate the E4bp4 PTM mutants influence transcription via E4bp4-binding consensus sequences in an episomal context. Based on these findings, it was decided to test, in a NK cell context, if the E4bp4 PTM mutants had any effect on the expression of endogenous genes known to strongly influence lymphoid cell development.

The mouse NK cell line MNK was transduced with the WT-form of E4bp4, the 5×-SUMO and S286-353A mutants. E4bp4 promoted Eomes expression in these cells and a similar level of expression was seen in the presence of both E4bp4 mutants (FIG. 9E). E4bp4 did not affect the transcription of Gata3 or Tbet, however, it was found to promote the expression of Notch1 and, strikingly, an even greater increase in expression was seen with the 5×-SUMO and S286-353A E4bp4 mutants (FIG. 9E). This provided evidence that SUMOylation and phosphorylation negatively regulate the transcriptional activation effect of E4bp4. These data show that removing these PTMs makes the E4bp4 protein both a more potent transcriptional activator or more potent transcriptional repressor depending on context. This observation provides an insight into the mechanism by which the mutant forms of E4bp4 enhance NK cell production to a greater extent than WT-form E4bp4 and suggests a previously unknown mechanism of NK cell development, namely, that E4bp4 might act on the Notch signalling pathway.

### Example 6—E4bp4 can Act Through Notch to Promote NK Cell Development

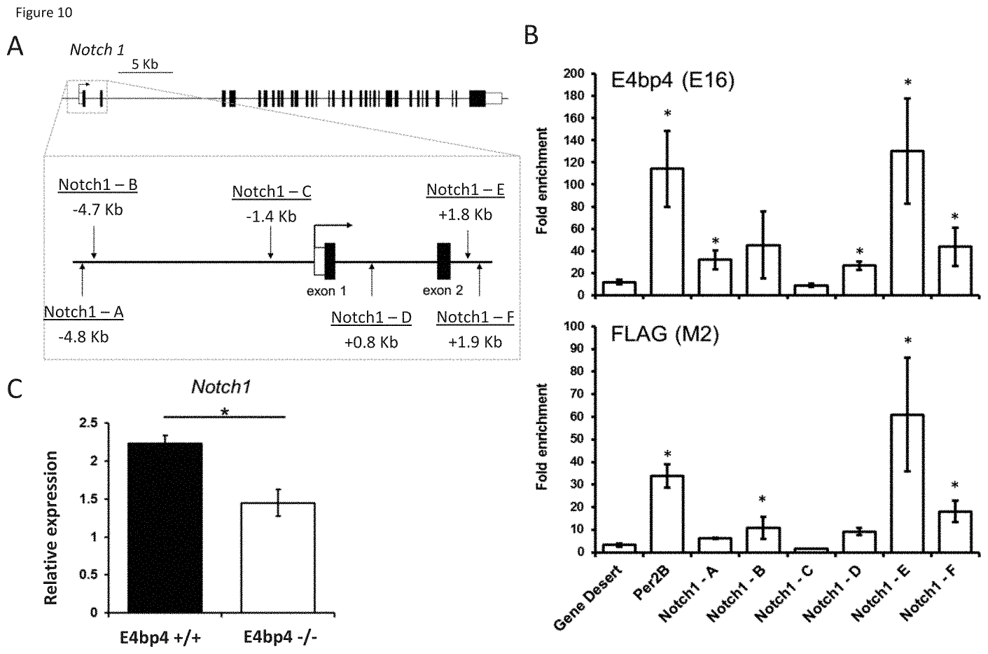

The effect of E4bp4 on the expression of Notch1, and whether this might potentially influence NK cell production, was investigated. Transient Notch signalling has previously been shown to induce the development of NK cells from Pax5^{-/-} pro-B cells and murine HSCs. First, it was examined whether Notch1 is a direct transcriptional target of E4bp4. Using chromatin immunoprecipitation (ChIP) it was determined whether E4bp4 could directly bind to the regulatory region of the Notch1 gene in vivo. MNK-1 cells were transduced with FLAG-tagged E4bp4 and protein-chromatin complexes were precipitated by either IgG, anti-FLAG or anti-E4bp4 antibodies. The regulatory regions around the transcriptional start site (TSS) of Notch1 were searched and six putative E4bp4 binding sites identified (FIG. 10A). E4bp4 binding was found to be highly enriched at predicted site E, to an even greater degree than the previously best characterised E4bp4 binding regulatory region found in the Per2B gene. In addition, sites A and B upstream of the TSS and sites D and F downstream of the TSS were also enriched in the E4bp4 immunoprecipitated samples (FIG. 10B). This indicated that E4bp4 binds to regulatory regions of Notch1 that could enhance its transcriptional activation. To further test the hypothesis that E4bp4 can regulate the expression of Notch1, it was postulated that a loss of E4bp4 should influence Notch1 expression in HPCs in vivo. The expression of Notch1 in E4bp4^{+/+} and E4bp4^{-/-} Lin⁻ bone marrow (BM) cells was compared and found that, in the absence of E4bp4, Notch1 expression was indeed significantly reduced as shown previously for another E4bp4 target gene, Id23 (FIG. 10C).

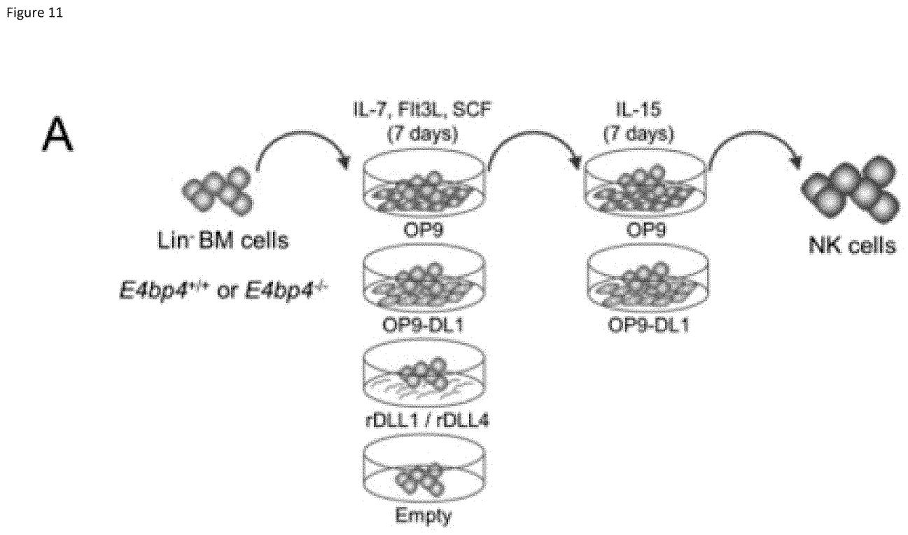

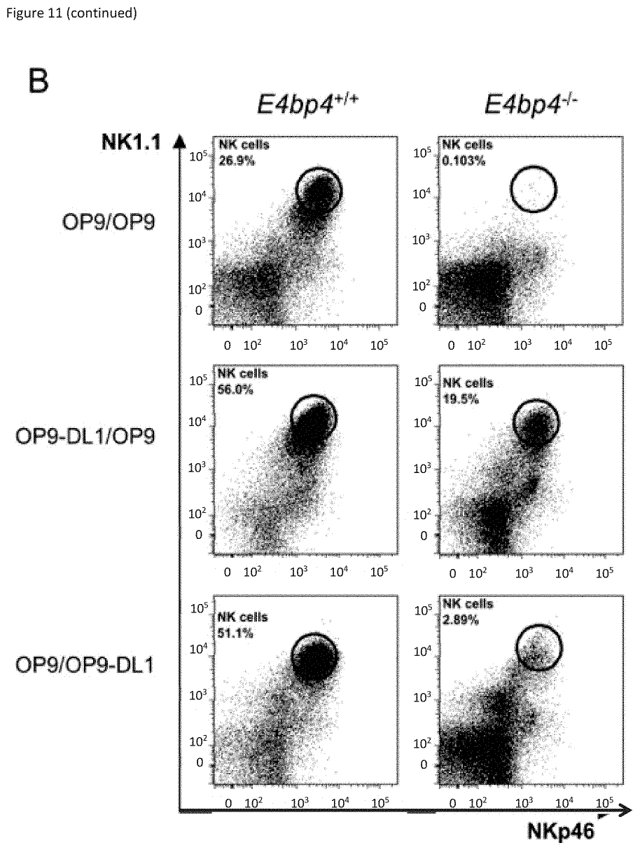

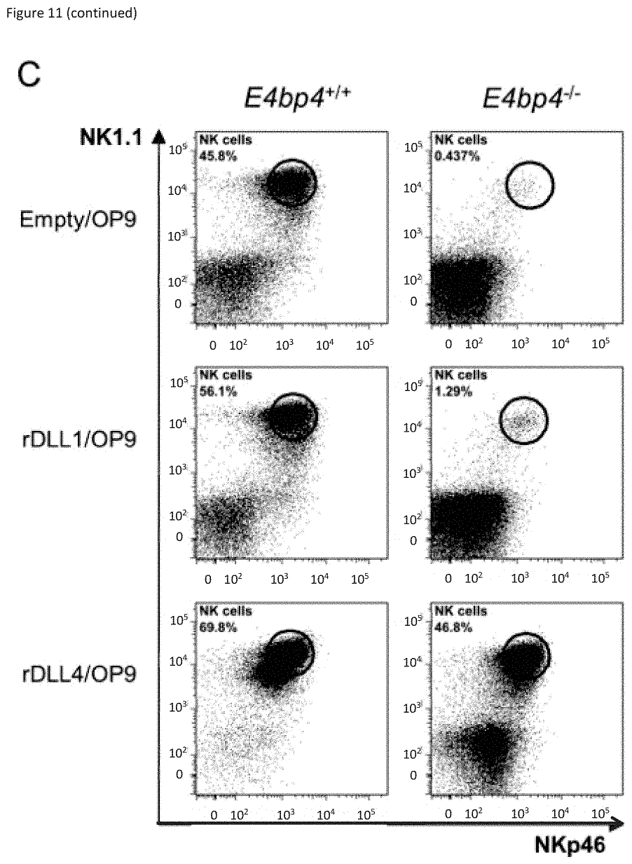

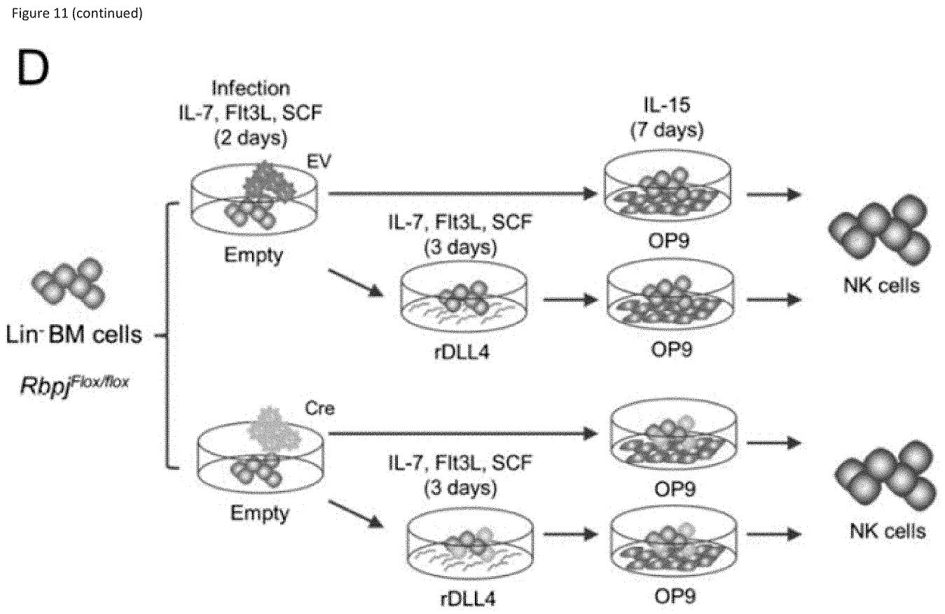

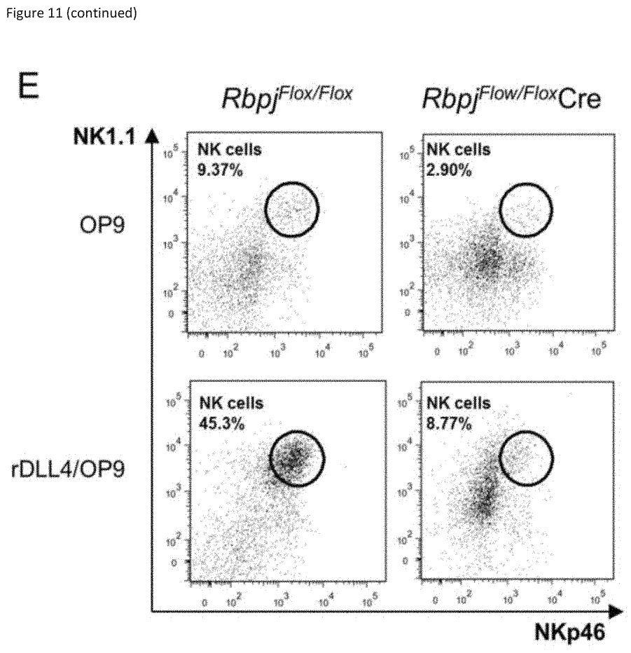

As E4bp4 appeared to regulate Notch1 expression, it was speculated that enhanced Notch signalling, at an early stage, could potentially affect the development of NK cells from E4bp4^{-/-} HPCs. To study NK cell development from HPCs in the presence or absence of Notch signalling, Lin⁻ BM cells were cultured on either OP9 stromal cells or OP9-DL1 cells (that express the Notch ligand delta-like 1) (FIG. 10C). Subsequently, cells were transferred onto fresh OP9 and cultured in the presence of IL-15 (FIG. 11A). When E4bp4^{-/-} cells were grown on control OP9, no NK cell development was observed, but when the E4bp4^{-/-} cells were grown on OP9-DL1 for the first part of the culture, remarkably, NK cell development was rescued (FIG. 11B). The same result was not observed when the cells were grown on OP9-DL1 for the second part of the culture (FIG. 11B). To eliminate the influence of stromal cells, tissue culture plates were coated with either recombinant delta-like ligand 1 (rDLL1) or rDLL4 protein. These plates were used for the first 7-day period of the culture (FIG. 11B). Some Delta-like ligands have previously been shown to efficiently induce Notch signalling when immobilised onto plastic surfaces. When cultured on empty plates, the E4bp4^{-/-} Lin⁻ BM cells did not develop into NK cells, but when grown on rDLL4-coated plates the production of NK cells could be dramatically rescued entirely in the absence of the critical transcription factor for NK cell development (FIG. 11C). Only a very partial rescue was observed when the cells were cultured on rDLL1-coated plates (FIG. 11C). Additionally, when E4bp4^{+/+} HPCs were cultured on rDLL4-coated plates, the level of NK cell production was increased compared to cells grown on uncoated plates (FIG. 11C).

It was then investigated whether abrogation of Notch signalling would have any direct influence on NK cell development by using a method engendering Cre-mediated deletion of the Rbpj gene. Recombination signal-binding protein J_{K} (RBP-J) is a transcriptional cofactor critical for the expression of target genes activated by the Notch signalling pathway. HPCs isolated from Rbpj^{flox/flox} mice were transduced by a lentivirus co-expressing Cre recombinase and truncated human CD2. The human CD2 expression served to mark all transduced cells. Following transduction, HPCs were cultured on OP9 cells with IL-15 or first cultured on rDLL4-coated plates for 3 days before transfer to OP9 plus IL-15 (FIG. 11D). Much reduced numbers of mature NK cells developed from those HPCs subject to Cre-deletion (FIG. 11E). This differential effect was greatly accentuated by pre-incubation with rDLL4 that selectively enhances NK cell development from HPCs (FIG. 11E). These data suggest that Notch signalling can play a role in the early stages of NK cell development and this action is as an integral part of the E4bp4-mediated transcriptional network that controls NK cell production.

### Example 7—the Combination of SR8278 and DLL4 Treatment Results in a Significant Increase in NK Cell Production Ex Vivo

The effect of combining REV-ERB inhibition and Notch ligand exposure on NK cell production was investigated.

HPCs were cultured in four sets of conditions in addition to control (non-treated) conditions: (i) treated with SR8278 on day 2 of culture (no recombinant DLL4, rDLL4); (ii) cultured on rDLL4 (no SR8278); (iii) cultured on rDLL4 and treated with SR8278 on day 0 of culture; or (iv) treated with SR8278 on day 0 of culture, cultured on rDLL4 from day 2. Aside from the different treatments, the culture conditions were otherwise identical to those previously described for NK cell production from HPCs.

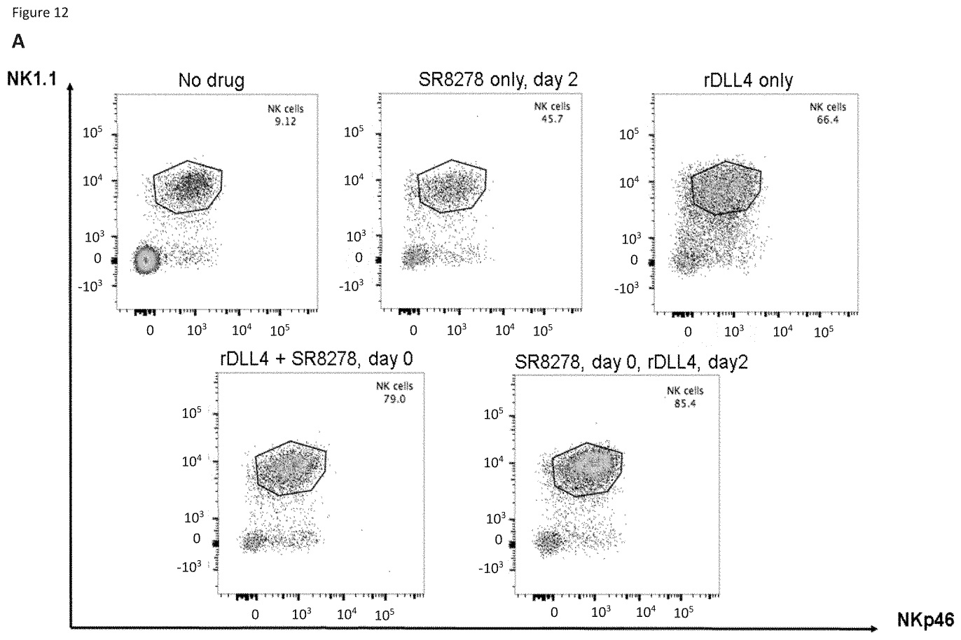

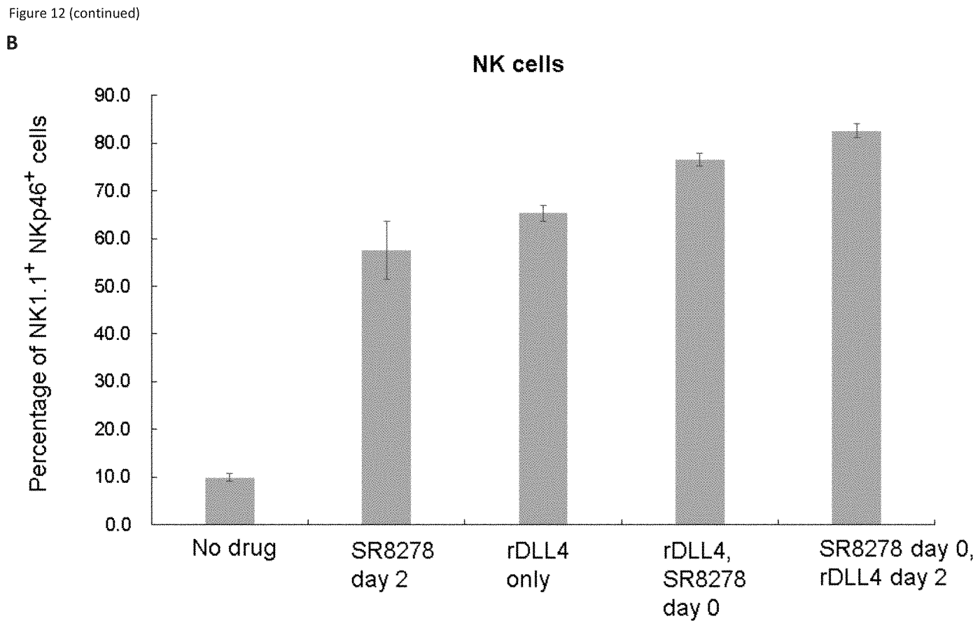

As shown in FIGS. 12A and B, the percentage of NK cells in the absence of both SR8278 and rDLL4 was low (less than 10%). The percentages (in FIG. 12B) are the average of separate triplicate experiments. The addition of SR8278 on day 2 of culture produced a significant increase in the % of NK cells produced (57.5%), as did culturing the HPCs on rDLL4 in the absence of SR8278 (65.3%). Culturing the HPCs on rDLL4 with SR8278 treatment on day 0 of culture elicited a further increase in NK cell production (77%). However, first incubating the HPCs with SR8278 on day 0 and then switching to culture on rDLL4 on day 2 had an even greater effect, with an average of 82.5% of the cells produced being NK cells (and in some repeats, over 85% of cells produced were NK cells).

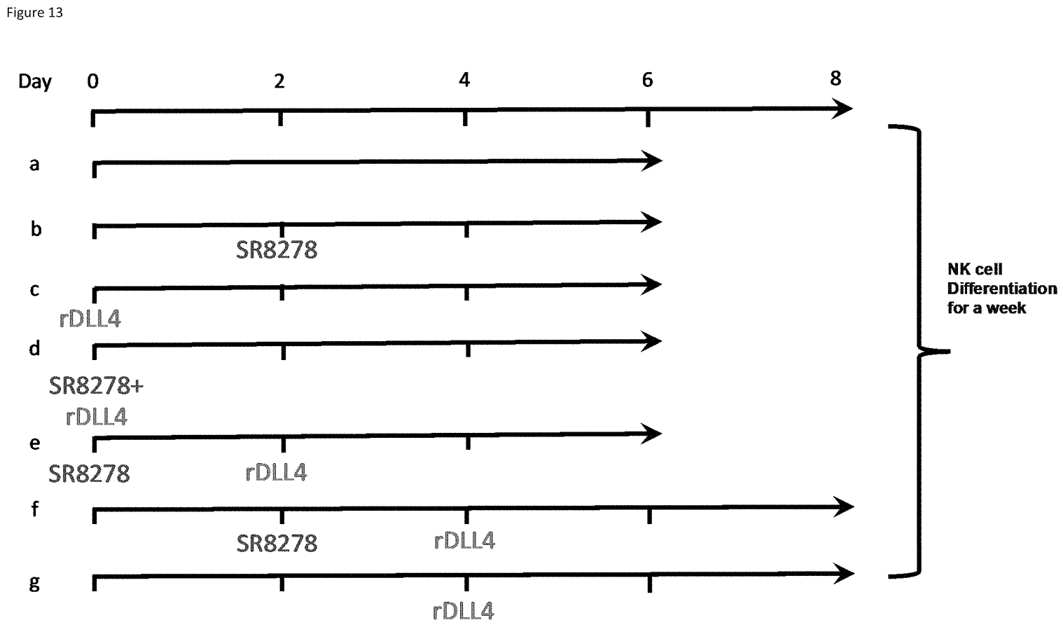

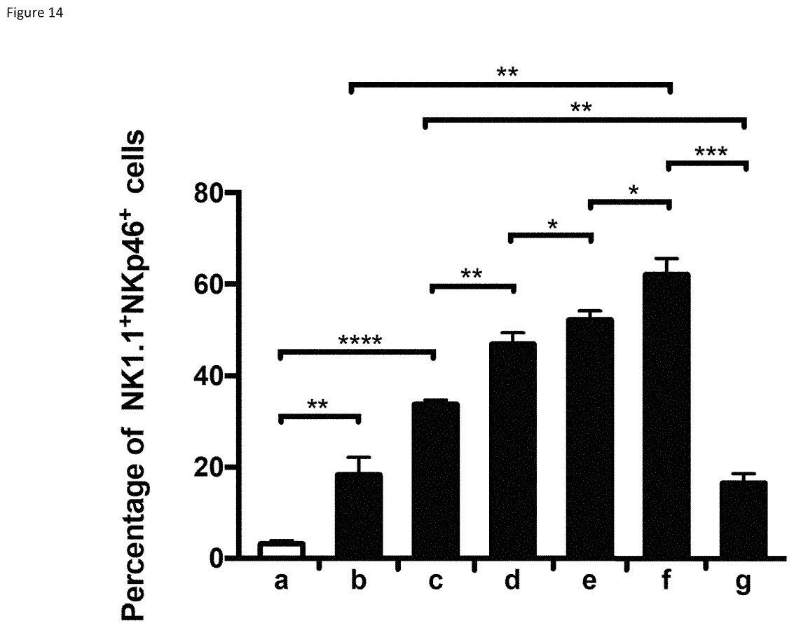

This experiment was repeated with an expanded set of culture conditions, as set out in FIG. 13. The results of this duplicate experiment are illustrated in FIG. 14. Again, this duplicate experiment demonstrated synergy between SR8278 and rDLL4. Condition f (SR8278 is added 2 days post isolation of bone marrow progenitors followed by exposure to rDLL4 2 days later) showed the strongest effect.

These data show that treatment of HPCs with SR8278 prior to (or simultaneously with) exposure to rDLL4 synergized to enhance NK cell production. E4bp4 is essential for the canonical production of NK cells. E4bp4 expression is increased by SR8278. Without being bound by theory, this can be explained by the induction of E4bp4 expression by SR8278, which in turn subsequently induces the expression of the Notch receptor. Later exposure to the Notch ligand, rDLL4 will maximize the effect on NK cell production.

Therefore, synergy between rDLL4 indicates that the rapid expansion of NK cells due to rDLL4 exposure following SR8278 treatment may well produce NK cells that have enhanced functionality in terms of their cytotoxicity, cytokine expression and survival post-transfusion to a recipient. Such NK cells would have a significant functional enhancement compared to existing techniques used to produce cells for adoptive transfer.

### Example 8—Exposure to Notch Ligand Greatly Accelerates the Production of Phenotypically Mature Human NK Cells

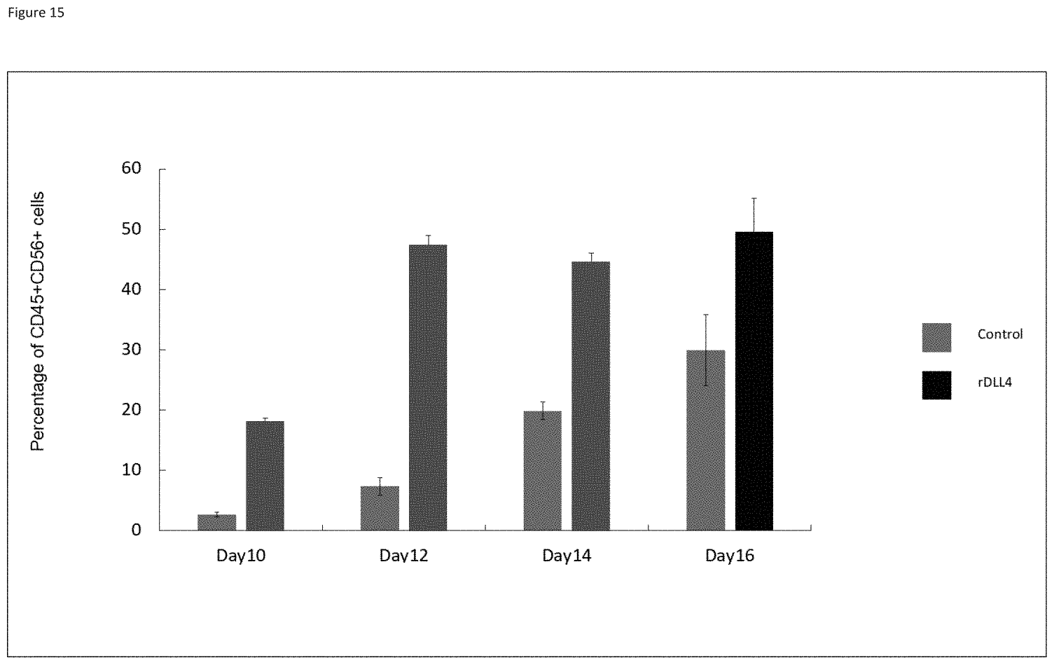

As is shown in FIG. 15, exposure of human CD34⁺ cord blood stem cells to the Notch ligand, rDLL4, significantly reduced the time required to produce a large population of mature human NK cells. In particular, exposure to rDLL4 resulted in the production of an NK cell population comprising almost 50% mature (CD45⁺/CD56⁺) NK cells after 12 days, compared with the control method in which only 30% mature NK cells were obtained even after 16 days. Populations with high percentages of mature NK cells are required by clinical protocols used for human NK cell production prior to transfusion to patients. Thus, the methods of the invention have the potential to greatly reduce any possible NK cell exhaustion phenotype which reduces the function of human NK cells after transfusion.

### Materials and Methods

### Mice

Wild type mice, E4bp4 heterozygous mice (E4bp4^{+/-}), E4bp4 knockout mice (E4bp4^{-/-}) and REV-ERB-α knockout mice (Rev-erb-α^{-/-}) were used. All mice were on a C57BL/6 background, between 6 and 12 weeks old and matched for age and gender. Rbpj^{flox/flox} mice were on a FVB background. All animal husbandry and experimental procedures were carried out according to UK Home Office regulations and local guidelines. E4bp4 mice were genotyped with the forward primer 5′CTCTGAGCTTGGCTGATGTG3′ (primer A) and reverse primer 5′GCTTCAAGTCTCCACCAAGC3′ (primer B) for detection of the wild type allele or 5′CCATGCTCCTGTCTTGATGA3′ for detection of the null allele.

### Cells and Cell Culture

OP9-GFP stromal cells were cultured in Iscove's Modified Dulbecco's Media (IMDM) (Sigma Aldrich) supplemented with 20% Fetal Bovine Serum (FBS), and Penicillin/Streptavidin (P/S). For experiments done on 96-well plates, OP9 stromal cells were plated at the concentration of 2000 cells/well and incubated at 37° C., 5% CO₂ for 1 day before the addition of HPCs. For experiments done on 24-well plates, OP9 stromal cells were plated at the concentration of 4000 cells/well and were incubated at 37° C. 5% CO₂ for 2 days prior to addition of HPCs. EL08.1D2 stromal cells were cultured in Minimum Essential Medium Eagle—Alpha Modification (Alpha-MEM) supplemented with 50% Myelocult M5300 (Stem Cell Technologies), 7.5% FBS, 50 μM f-Mercaptoethanol, 1 μM Hydrocortisone and 1% P/S (Sigma Aldrich). For human CD34⁺ progenitor cell experiments, EL08.1D2 were irradiated at 3000 rads/30 Gy and plated in 96-well EmbryoMax Gelatin (Millipore)-coated plates at the concentration of 20,000 cells/well. There were cultured at 32° C., 5% CO₂ overnight, before CD34⁺ cells were transferred onto them.

### Mouse HPC Isolation

Lineage negative HPCs were purified from mouse bone marrow by crushing the leg bones in Phosphate-buffered Saline (PBS) with 2% fetal calf serum (FCS) (STEMCELL Technologies), topped up to 40 ml with magnetic-activated cell sorting (MACS) buffer (PBS, 2 mM EDTA, 0.5% BSA, sterile and filtered) and centrifuged at 800 g for 2 minutes. The cells were resuspended in PE-conjugated cocktail, (20 μl of anti-B220 (RA3-6B2), anti-mouse CD2 (RM2-5, anti-Ter119 (TER119) and anti-NK.1.1 (PK136) and 5 μl of anti-CD11b (M1/70) and anti-GR-1 (RB6-8C5) antibodies (all from Bioscience)) incubated for 5 minutes at 4° C., centrifuged and resuspended in anti-PE microbeads for 15 minutes at 4° C. Cells were washed in MACS buffer and passed through MACS columns. This allowed negative selection of HPCs. Following lineage depletion, 50 μl of the cells was analysed using flow cytometry to check for purity.

In Vitro Development of NK Cells from HPCs

The HPCs were plated and cultured in 24-well plates at a concentration of 5×10⁵ HPCs/well in 1 ml of complete cytokine medium (Dulbecco's modified eagle medium (DMEM) (Sigma Aldrich), 10% FCS, 50 μM β-Mercaptoethanol, 10 ng/ml Flt3-ligand (Flt3L) (R&D Systems), 10 ng/ml IL-7 (R&D Systems), 100 ng/ml stem cell factor (SCF) (R&D Systems) and 1% P/S) for 2 days at 37° C., 5% CO₂. The HPCs were then transferred onto OP9 cells at 4500 cells/well for 96-well plate experiments and 3×10⁴ cells/well for 24-well plate experiments in mouse NK cells differentiation medium (Alpha-MEM (Sigma Aldrich) plus 20% FCS, 1% P/S and 30 ng/ml IL-15). Cells were left in culture at 37° C., 5% CO₂ for 7 days with a change of mouse NK cells differentiation medium at day 3 or 4.

### In Vitro Development of Human Umbilical Cord Blood Progenitor Cells

CD34⁺ umbilical cord blood progenitor cells were provided by Anthony Nolan Research Institute, University College London. These cells were isolated from whole cord blood and were cryopreserved in liquid nitrogen for storage and transport. Cells were thawed, counted and then plated on EL08.1D2 plates prepared previously at a concentration of 1000 cells/well in human NK cells differentiation medium (Alpha-MEM plus 20% Human AB serum (Invitrogen), 50 μM β-Mercaptoethanol and 1% P/S along with 5 ng/ml human-IL-3 (Peprotech), 20 ng/ml human-IL-7 (Peprotech), 10 ng/ml human-Flt3-L (Peprotech), 20 ng/ml human-SCF (Peprotech) and 10 ng/ml human-IL-15 (Peprotech). Note that human-IL-3 is only needed for the first week of culture. Cells were left in the culture at 37° C., 5% CO₂ for 14 or 16 days with a change of human NK differentiation medium at day 7 and 12.

### Flow Cytometry

Cells to be analysed by flow cytometry were passed through 40 μm cell strainers to remove clumps and washed with PBS buffer, centrifuged at 800 g for 2 minutes and resuspended in 100 μl fluorescent activated cell sorting (FACS) buffer (PBS plus 1% BSA) with appropriate fluorochrome conjugated antibodies at a dilution of 1 in 300. Cells were stained with the following antibodies, all of which were anti-mouse and are from eBioscience unless specified: 2B4 (clone m2B4(B6)458.1; BioLegend), CD2 (RM2-5), CD3 (17A2), CD11b (M1/70), CD19 (1D3), CD27 (LG.7F9), CD122 (TM-b1), CD127 (A7R34), B220 (RA3-6B2), ckit (ACK2), Flt3 (A2F10), Gr1 (RB6-8C5), NK1.1 (PK136), Sca1 (D7), Ter119 (TER119), NKp46 (29A1.4) anti-human CD45 (H130), anti-human CD2 (RPA-2.10) and anti-human CD56 (CMSSB). The lineage cocktail contained B220, CD2, CD11b, Gr1, NK1.1, and Ter119. Cells were stained in the dark at 4° C. for 30 minutes and then washed with 2 ml FACS buffer, centrifuged and resuspend in 300 μl FACS buffer plus Propidium Iodide (PI) also at a dilution of 1 in 300. Flow cytometry was performed using LSRFortessa™ cell analyser (Becton Dickinson Bioscience), sorted using FACSAria (Becton Dickinson) as indicated and full data analysis was done using FlowJo Software.

### Polymerase Chain Reaction (PCR)

Individual PCR reactions contained 200 μM dNTPS, 1 μM forward primer (Primer A), 1 μM reverse primer (Primer B or C) and 0.5 U Taq polymerase. PCR reactions were set to the following conditions: 94° C. for 3 minutes (1 cycle); 94° C. for 30 seconds, 59° C. for 3 seconds, 72° C. for 45 seconds (40 cycles); 72° C. for 3 minutes (1 cycle); hold at 4° C.

### DNA Electrophoresis

DNA electrophoresis was performed using 1% agarose (Sigma) dissolved in TAE buffer plus 500 ng/ml Ethidium Bromide (Sigma). DNA obtained from PCR reactions was analysed by gel electrophoresis was performed at 100 volts for approximately 45 minutes. Gels were imaged using EC3 Imaging System (Ultra Violet Products Ltd).

### RNA Purification

RNA was extracted using Qiagen RNeasy Micro Kit according to the manufacture's protocol (Qiagen). Centrifugation was done at 8000 g for 15 seconds and the flow through discarded. Briefly, 350 μl of Buffer RLT+10% β-Mercaptoethanol were added to the harvested cells. RNA was further precipitated using 300 μl of 70% ethanol and transferred to RNeasy MinElute Spin Column and centrifuged. Next, 350 μl of buffer RW1 was added to the MinElute Spin Column and centrifuged. This was followed by the addition of 10 μl of DNase I (Qiagen) and 70 μl Buffer RDD (Qiagen) and left at room temperature for 15 minutes. 350 μl of Buffer RW1 was added to wash off DNase I and centrifuged. 500 μl of Buffer RPE was then added to the column and centrifuged, followed by the addition of 500 μl of 80% ethanol and centrifuged for 2 minutes. Finally, 14 μl of RNase-free water was added to elute the RNA and the column was spun for 1 minute at full speed. The concentration of RNA in each sample was measured using Nanodrop, and all samples were diluted to the same working concentration.

Reverse Transcription (Conversion of RNA into cDNA)

Reverse transcription was performed using Transcriptor First Strand cDNA Synthesis kit (Roche). Following the manufacturer's protocol, a template-primer mixture for one 20 μl reaction was prepared, where all reagents are provided in the kit: RNA (1 μg to 5 μg), 2 μl Random Hexamer Primer, top the reaction up to 13 μl with water (PCR-grade). Next, the template-primer mixture was denatured by heating the tube for 10 minutes at 65° C. to remove RNA secondary structures. To that template-primer mixture, 4 μl of Transcriptor Reverse Transcriptase Reaction Buffer, 0.5 μl of Protector RNase Inhibitor, 2 μl of Deoxynycleotide Mix and 0.5 μl of Transcriptor Reverse Transcriptase was added. The reagents was mixed and placed In a thermal block cycler with the following settings: 25° C. for 10 minutes; 55° C. for 30 minutes; 85° C. for 5 minutes and store at 4° C.

Quantification of Targeted Expressed RNA Using Realtime qPCR

| Temperature (° C.) | Time |
|---|---|
| 95 | 20 minutes |
| 95 | 3 seconds |
| 96 | 30 seconds |

### Conditions Used for RT-qPCR

A standard curve was constructed using splenocytes cDNA diluted to 1, 1:10, 1:100, 1:1000 and 1:10000. To the 2 μl of cDNA produced in the previous step, 5 μl Taqman master mix (Applied Biosystem), 0.5 μl of Taqman gene expression assay kit of Hprt, Nfil3, Id2 or Eomes (Applied Biosystem) and 2.5 μl of RNase-free water. The program used is shown in Table 1 and the reaction was run for 47 cycles.

Analysis of SUMOylation In Vivo Using 6his-SUMO HeLa Cells

The HeLa cell lines 6His-SUMO-1, 6His-SUMO-2, 6His-SUMO-3 and parental HeLa cells were transfected with pCMV-E4BP4 or pCMV (empty vector). After an input sample was removed, the remaining cells were lysed in 6 M Guanidinium-HCl, before Ni²⁺ affinity purification. Ni²⁺ NTA agarose beads (Qiagen) were incubated with cell lysates overnight (O/N) at 4° C. Samples were washed with 8 M Urea and His-tagged proteins were eluted with 200 mM imidazole.

### Immunoprecipitation of FLAG-E4bp4

E4bp4 cDNA was cloned into the pCMV-script vector (Promega) using primers to incorporate a 5′ FLAG tag after the start codon. HeLa cell lines 6His-SUMO1, 6His-SUMO2 and 6His-SUMO3 were transfected with pCMV-FLAG-E4bp4 or pCMV (empty vector). Cells were lysed using a two-step lysis protocol and lysates were incubated with anti-FLAG M2 Affinity Gel (Sigma Aldrich) O/N at 4° C. Samples were centrifuged and the supernatant was removed. Each sample was washed with TBS (50 mM Tris-HCl, 50 mM NaCl, pH 7.4) before elution of purified material by Laemmli sample buffer.

### Western Blotting

Cell lysates and protein samples were mixed 1:1 with Laemmli sample buffer and reduced by boiling in 5% β-mercaptoethanol. Samples were separated on 8% polyacrylamide gels, transferred to PVDF membranes and membranes were probed with primary antibodies against: E4bp4 (C18; Santa Cruz Biotech), SUMO2/3 (AbCam), FLAG (M2; Sigma Aldrich), 6×-His (4D11; AbCam), α-Tubulin (DM1A; eBioscience), RanGAP1 (AbCam), Histone H3 (AbCam). Appropriate HRP-conjugated secondary antibodies (Abam) were used with Western Lightning® Plus-ECL detection reagents (Perkin Elmer) to determine chemiluminescence. Images of exposed blots were digitally acquired using the ChemiDoc™ XRS+system (Bio-Rad).

### Site Directed Mutagenesis

Single base pair mutations were made in the E4bp4 cDNA (K10R, K116R, K219R, K337R, K394R, S286A, S301A, and S353A), in the pCMV-script expression vector, using the QuikChange® XL site-directed mutagenesis kit (Agilent) and appropriately designed primers. Each mutant was also cloned into the pMSCV-IRES-hCD2 retroviral expression vector. The 5×-SUMO and S286-353A mutants were also cloned into the lentiviral expression vector pCSGW.

### Mass Spectrometry Analysis

293T cells were transfected with pCMV-FLAG-E4bp4 and pCDNA3-VP35 using Lipofectamine 2000 (Life Technologies). The presence of the Ebola virus VP35 protein, helped to enhance recombinant protein expression. E4bp4 was immunoprecipitated from whole cell lysates using anti-FLAG M2 Affinity Gel (Sigma Aldrich). Bound material was eluted using 150 ng/μl FLAG peptide (Sigma Aldrich). Purified E4bp4 was concentrated using vacuum centrifugation and resuspended in 100 mM ammonium bicarbonate pH 8. Samples were reduced with 5 mM dithiothreitol and 14 mM iodoacetamide was used to label reduced cysteines. Proteomics-grade trypsin (Promega) was used to digest the E4bp4 protein for 6 h at 37° C. For SUMOylated peptide analysis samples were sequentially digested with GluC (Roche) for 6 h at RT.

Phosphorylated peptides were enriched using TiO₂ (GL sciences) and phosphopeptides were eluted with 150 mM ammonium hydroxide and 50% acetonitrile (v/v) (Millipore). Peptides were chromatographically resolved on an Ultimate 3000 RS-LC-nano System (Dionex), with an Acclaim PepMap100, C18 stationary phase (Thermo Fisher). Real-time tandem mass spectra were acquired on an LTQ Velos Pro linear ion trap (Thermo Scientific). Initial phosphopeptide identification from the LC-MS/MS data was performed using a Sequest search in Proteome discoverer 1.3 (Thermo Fisher) against the Mouse Uniprot database (accessed: 19/08/14) including dynamic side-chain modifications including phosphorylation (+79.966) on serine, threonine, and tyrosine residues. Putative phosphopeptides were then validated using an algorithm for phosphorylation site identification and manually assessed for accuracy.

The SUMOylated peptides were analysed using a mixture of data-dependent acquisitions and targeted MS/MS scans to search for peptides containing putative sites of SUMOylation based on a SUMO tag of GGTQQFV. Specifically, targeted MS/MS scans were always collected for the following m/z values, which correspond to E4bp4 peptides predicted to have SUMOylated lysines: K10 m/z 2+=531.99; K116 m/z 2+=796.49; K219 m/z 2+=853.54; K337 m/z 2+=825.52).

### Cycloheximide Time Course

3T3 cells were stably transduced with pMSCV-E4bp4-hCD2 (or E4bp4 mutant versions) and after 48 h cells were bulked sorted for high hCD2 expression. Transduced cell lines were incubated with 50 μg/ml cycloheximide (Sigma-Aldrich) for 0-16 h. Nuclear extracts were prepared for all samples using NE-PER extraction regents (Pierce).

### Luciferase Assay

E4bp4 transcriptional activity was analysed using the pGL3-E4bp4-CS vector24, containing the Firefly luciferase reporter gene and the Dual-Luciferase® reporter assay system (Promega). 3T3 cells were transfected with pMSCV-E4bp4-hCD2, pGL3-E4bp4-CS (or empty vector) and pRL-CMV (normalisation control). After 48 h, cells were lysed and the Dual-Luciferase® reporter assay system was used to determine Luciferase activities.

### Quantitative PCR

MNK-1 cells were transduced with pCSGW-E4bp4 (or E4bp4 5× SUMO or E4bp4 S286-353A mutants). RNA was isolated using the RNeasy mini kit (Qiagen) and cDNA was synthesised using 1st Strand cDNA synthesis kit (Roche). QPCR was performed using TaqMan (Life Technologies) assays for Hprt1 (Mm00446968_m1), E4bp4 (E4bp4; Mm00600292_s1), Eomes (Mm01351985_m1), Gata3 (Mm00484683_m1), Notch1 (Mm00435249_m1) and T-bet (Tbx21; Mm00450960_m1). Samples were analysed using an Applied Biosystems 7500 Fast Real-Time PCR system. Ct values from samples were compared with a standard curve made from a known concentration of plasmid DNA (Eomes, T-bet, Gata3) or cDNA from a known number of murine splenocytes (Notch, Hprt1). The expression of all genes was normalised to Hprt1.

### Chromatin Immunoprecipitation

Regulatory regions of Notch1 were searched for putative E4bp4-binding sites (T(T/G)A(T/C)GTAA) using MatInspector (Genomatix). MNK-1 cells were transduced with a lentivirus expressing FLAG-E4bp4 and ChIP immunoprecipitation performed. Briefly, protein-DNA complexes were immunoprecipitated with IgG (EMD Millipore), M2 antibody to FLAG (Sigma-Aldrich), or polyclonal E16 antibody to E4bp4 (Santa Cruz Biotechnology, Inc.). Purified DNA was amplified using SYBR Select master mix (Life Technologies) and primers designed to recognise putative E4bp4-binding regions (Table 2).

TABLE 2
Primers for the amplification of putative
E4bp4-binding regions in Notch1 locus

| | Forward | Reverse | |
|---|---|---|---|
| | primer | primer | Amplicon |
| Region | (5′-3′) | (5′-3′) | (bp) |
| Notch1 A | CTATAT | GAAGTA | 168 |
| | TTTTGC | CGAAGC | |
| | CTTGAC | ATGCTT | |
| | AGCTAA | GC | |
| | AGG | | |
| Notch1 B | CACATC | GACTGA | 170 |
| | TGTGAG | CTAAAC | |
| | CTATTT | TAACAT | |
| | TTGG | TCCCAC | |
| Notch1 C | CTCAGA | CACTTG | 144 |
| | AACTGG | CAGTCA | |
| | CCTCAA | GGCGTT | |
| | GC | C | |
| Notch1 D | CACGCC | GTAACC | 135 |
| | ATCTTA | AACTGC | |
| | AAGAGC | ACTCTT | |
| | TC | CTCC | |
| Notch1 E | CACCAA | GAGTGC | 144 |
| | GAATTC | AGTCAC | |
| | CCAGGA | GTGCTG | |
| | G | AC | |
| Notch1 F | CTCAGA | CGTGTG | 160 |
| | CTCTCT | GAGCTA | |
| | CGGTAA | CTCTGG | |
| | GTGTC | C | |

In Vitro Development of NK Cells from Transduced Lineage Negative Bone Marrow Cells

Lin⁻ BM cells were isolated from mouse leg bones and cultured in DMEM supplemented with 10% FCS (Stemcell Technologies), 50 μM β-mercaptoethanol (Gibco), 10 ng/ml Flt3L (PeproTech), 10 ng/ml IL-7 (PeproTech), and 100 ng/ml SCF (PeproTech). After 48 h cells were transduced by spinfection at 700 g and 20° C. for 45 min with 8 g/ml Polybrene. For positive controls, cells were transduced with pMSCV-IRES-hCD2, containing WT E4bp4. Transduced cells were cultured for 72 h before being resuspended in α-MEM supplemented with 20% FCS, f-mercaptoethanol, and 30 ng/ml IL-15 (PeproTech) and re-plated onto OP9 stromal cells for a further 7 days of culture.

To investigate Notch1 signalling, Lin⁻ BM cells were cultured on OP9, OP9-DL1 or plates pre-coated with rDLL1 (R&D Systems) or rDLL4 (R&D Systems). Plates were pre-coated with 10 μg/ml rDLL1/rDLL4 for 3 h at room temperature. Cells were incubated in α-MEM supplemented with 10% FCS, β-mercaptoethanol, 1 mM Sodium Pyrvuate, 25 mM HEPES and for the first 7 days with Flt3L, IL-7, and SCF. Cells were incubated for another 7 days on either on OP9 or OP9-DL1 in the presence of IL-15.

Rbpj^{flox/flox} Lin⁻ BM cells were lentivirally transduced on the day of isolation with pCSGW-Cre (or empty vector) that co-expresses hCD2. Cells were transduced by spinfection and were cultured for two days in the presence of Flt3L, IL-7, and SCF. Cells were either transferred onto rDLL4-coated plates for three more days of culture or transferred directly on OP9. Cells were cultured on OP9 for 7 days with IL-15. For flow cytometry analysis, all cells were gated for hCD2 expression to identify the population transduced with lentivirus.

### Statistical Analysis

Statistical analysis was done using Mann-Whitney test in Graph Pad Prism 7

### Discussion

NK cells are lymphocytes that are capable of producing cytokines, influencing other immune cells as well as killing cancerous, pathogen-infected or damaged cells directly. Due to these properties, researchers are interested in boosting the number of NK cells in order to enhance cytotoxicity against cancerous or pathogen-infected cells. NK cells develop from HSCs in the bone marrow and are controlled by a tightly regulated process involving various transcription factors and cytokines. E4bp4 is the most critical gene regulating NK cell development. E4bp4 has a profound effect on NK cell production despite there being only a relatively small increase in E4bp4 mRNA levels during NK cell development. Little is known about any means that exist to control the activity of E4bp4 protein. The ability to control E4bp4 expression would have highly significant implications for the development and production of NK cells.

The present inventions have previously demonstrated that upon administration of SR8278, the production of NK cells in NK cell production assays increases more than 2-fold. In the assays conducted, the optimum time to add SR8278 to the HPC culture for in vitro NK cell production was at Day 2 and the optimum dose was 10 μM. The addition of SR8278 was also found to significantly increase human NK cell development from human HPCs cultured in vitro.

The present inventors have demonstrated that the E4bp4 protein has multiple SUMO modifications and is predominantly modified by the SUMO2/3 isoform. K219 as a site an endogenous site of SUMO modification. The MS analysis of the E4bp4 protein also revealed it is phosphorylated at three sites; S286, S301 and S353.

E4bp4 plays a central role in NK cell development where its expression in CLPs is required to commit developing cells to the NK lineage. The inventors have shown that the SUMOylation and phosphorylation sites of E4bp4 have a dramatic influence both on the activity of E4bp4 and on NK cell development. When comparing the activity of WT-form E4bp4 to mutant forms that lack SUMOylation or phosphorylation sites, the mutant forms were found to consistently promote greater levels of NK cell production. The two types of post-translational modification reported on herein, act in a similar manner to negatively regulate the activity of E4bp4 during NK cell production. E4bp4 was previously demonstrated to be a limiting factor for NK cell development, as transduction of E4bp4+/+Lin− BM cells with E4bp4 caused increased levels of NK cell production3,5. Transducing E4bp4+/+ cells with mutant forms of E4bp4 also increased NK cell production but to a greater extent than the WT-form E4bp4. This demonstrated that SUMOylation and phosphorylation both negatively regulate the activity of E4bp4, as removing the sites of these modifications increases E4bp4 activity and ultimately NK cell production. As E4bp4 is critical for NK cell development, it is highly likely that its activity is tightly controlled by multiple mechanisms as aberrant activity could lead to defective haematopoiesis. There are no other well characterized examples where the PTM of a single transcription factor can have such a dramatic effect on a complex biological process such as lineage development.

SUMOylation and phosphorylation were both found to suppress the transcriptional activity of E4bp4 regardless of whether it was activating or repressing transcription. The effect of WT-form E4bp4 and the PTM mutant forms on the expression of transcription factors known to regulate lymphoid commitment was compared. The remarkable outcome of this comparison was that Notch1 expression was upregulated in the presence of WT-form E4bp4 but that expression was significantly further enhanced in the presence of both the E4bp4 5×-SUMO mutant and the S286-353A phosphorylation mutant.

Notch signalling activated by extrinsic ligands has previously been suggested to have a role in the development of both murine and human NK cells. It must act transiently during the early phase of NK cell development, as prolonged signalling induces T cell development. The inventors have shown that Cre-mediated deletion of Rbpj in HPCs results in impaired NK cell development, which is the first report of an intrinsic role for Notch in NK cell development. In particular, the data herein demonstrates that Notch signalling significantly enhances NK cell development. In particular, mice with conditional deletion of Notch1 in their haematopoietic cells do not have significantly reduced numbers of NK cells. This could be because the E4bp4-mediated transcriptional network in the conditional knockout cells remains intact and can compensate for the lack of Notch1. Early stages of NK cell development may be somewhat impaired with Notch1 deleted, but homeostatic processes could lead to the accumulation of normal numbers of peripheral NK cells in steady state conditions.

Like Notch, E4bp4 is required during early lymphocyte development and must be expressed in CLPs for them to commit to the NK lineage, however it is dispensable for the maturation and functionality of mature NK cell8. E4bp4 and Notch1 are also important for the development of other innate lymphoid cell types, for example, lymphoid tissue inducer (LTi) cells, where Notch signalling is required to engage the LTi developmental pathway but needs to be turned off later to avoid diversion to T cell fate. The inventors have found that E4bp4 transcriptionally regulates Notch1 as it binds directly to regulatory regions of the Notch1 gene and in the absence of E4bp4, Notch1 expression was reduced in HPCs. E4bp4 most prominently bound to a region 1.8 Kb upstream of the TSS of Notch1 in a similar manner to other transcription factors, such as DLX5 and ERβ, known to regulate Notch1 expression. Strikingly, increased Notch signalling during the early part of NK cell development alone was sufficient to completely rescue the development of NK cells from E4bp4^{-/-} progenitor cells. This strongly suggests that Notch1 acts downstream of E4bp4 during NK cell development. This rescue was only achieved when Notch ligands were present during the early stage of NK cell development. The rescue from E4bp4^{-/-} progenitors was achieved using both DLL1 expressing OP9 stromal cells and rDLL1 and rDLL4 proteins immobilised on plastic plates. The rDLL4 had a much greater effect than the rDLL1 on NK cell development as DLL4 binds Notch1 with much higher affinity than DLL1.

As Notch1 ligands are expressed in the bone marrow microenvironment, it appears that the availability of Notch signalling at the appropriate time can drive NK cell development. Similarly to E4bp4, Notch1 has also been found to regulate the expression of Eomes48, which could be a further means by which Notch1 enhances NK cell development via the E4bp4-regulated pathway. Thus, the data shown here for NK cells is indicative of a central mechanism linking extrinsic signals via E4bp4 to direct transcriptional control of all ILC production.

In sum, control of E4bp4 expression and/or activity by extrinsic stimuli such as Notch ligands has significant implications for the production of human NK cells for use in immunotherapy. The conventional methods for the production of NK cells from various sources (e.g. induced-pluripotent stem cells and umbilical cord blood stem cells) involves the use of cytokines and stromal cells to commit the cells to the NK lineage, but influencing E4bp4 expression and/or activity, and/or Notch signalling could provide a simple strategy to enhance the process. Thus, the manipulation of E4bp4 activity and/or expression and/or Notch signalling, either alone, and particularly in combination, has potential utility in the production of future NK cell immunotherapeutic products, including the direct mobilisation of NK cell production in vivo as immunotherapy.

### Example 9—Inclusion of a Pre-Differentiation Step with DLL4 Increases CD16⁺ NK Cell Production

CD34⁺ HPCs were isolated from human umbilical cord blood and frozen prior to testing. Isolated CD34⁺ HPCs were thawed, allowed to recover for 2 days. The HPC were then cultured in a non-differentiation medium for a period of 6 days, wherein the HPCs were cultured in the presence or absence of DLL4 peptide. The presence of DLL4 peptide was via a DLL4-coated plate.

Following this pre-differentiation step, the pre-differentiation HPC population was transferred to a fresh culture vessel and cultured in a differentiation medium for a further 20 days. At the end of this differentiation period, the cells were analysed by flow cytometry to quantify CD56 and CD16 expression.

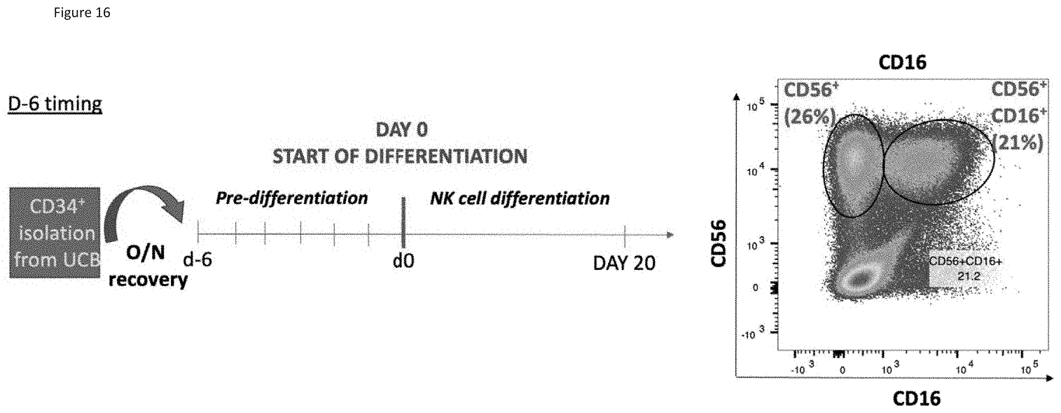

As shown in FIG. 16, inclusion of DLL4 during a pre-differentiation step increased CD16⁺ expression, with a significant increase to 56% of the resulting NK cells being CD16′.

### Example 10—Inclusion of DLL4 Further Increases CD16⁺ NK Cell Production

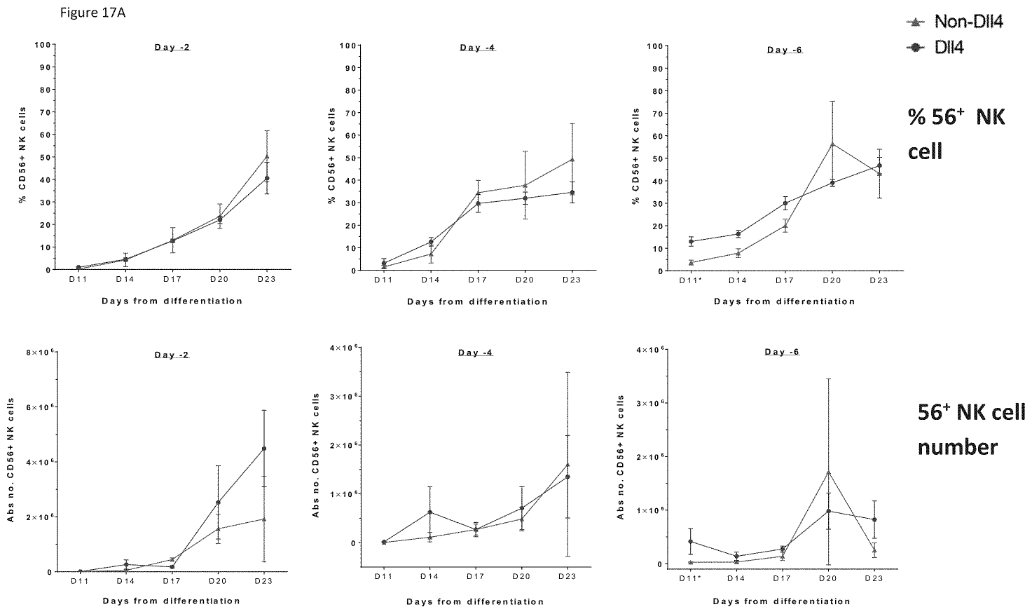

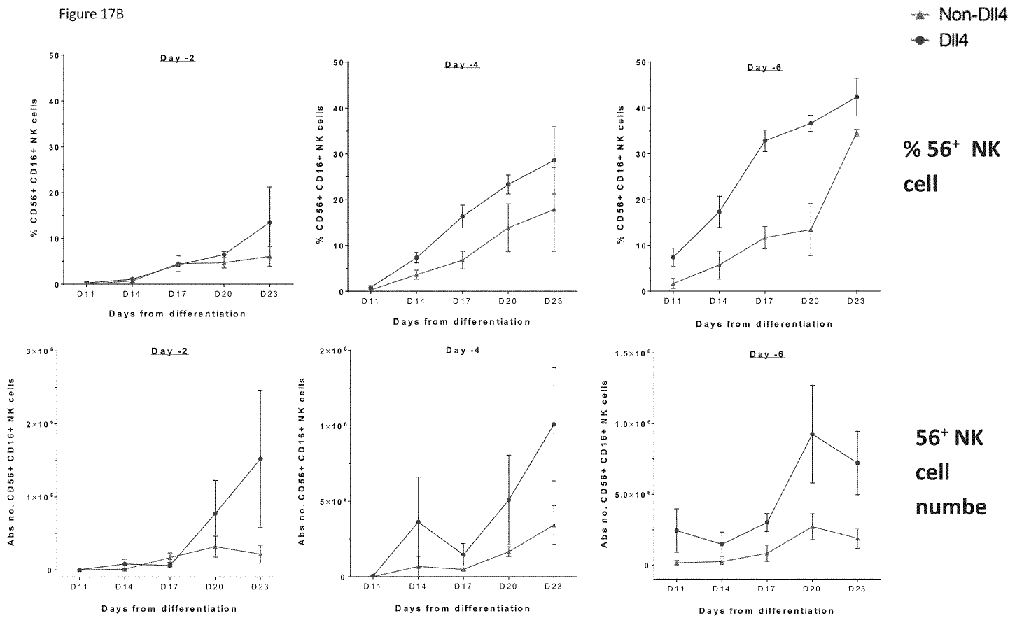

The experiment in Example 9 was repeated in which the length of the pre-differentiation period in the presence/absence of DLL4 was investigated. Pre-differentiation periods of 2, 4 and 6 days were tested. FIG. 17A shows the results expressed as a time course and shows the percentage (upper panels) and absolute number (lower panels) of CD56⁺ cells. FIG. 17B shows the results expressed as a time course and shows the percentage (upper panels) and absolute numbers (lower panels) of CD56⁺ CD16⁺ cells either with (•) DLL4 or without (▴) DLL4. These results demonstrate that, whilst there was no significant effect on the total number of NK cells (CD56⁺, FIG. 17A), the number of CD16⁺ NK cells further increased when DLL4 was combined with the pre-differentiation step (FIG. 17B).

### Example 11—Omission of IL-3 Further Increases CD16⁺ NK Cell Production when Cells are Cultured with DLL4

CD34⁺ HPCs from human umbilical cord blood were treated with either DLL4 or with vehicle control only (CTRL) for 4 days prior to transfer to stromal cells (+EL08). Controls without EL08 (−EL08) were also tested. IL-3 was added (+IL-3) or withheld (−IL-3) and the percentage of CD56⁺ CD16⁺ NK cells produced was measured over time of differentiation.

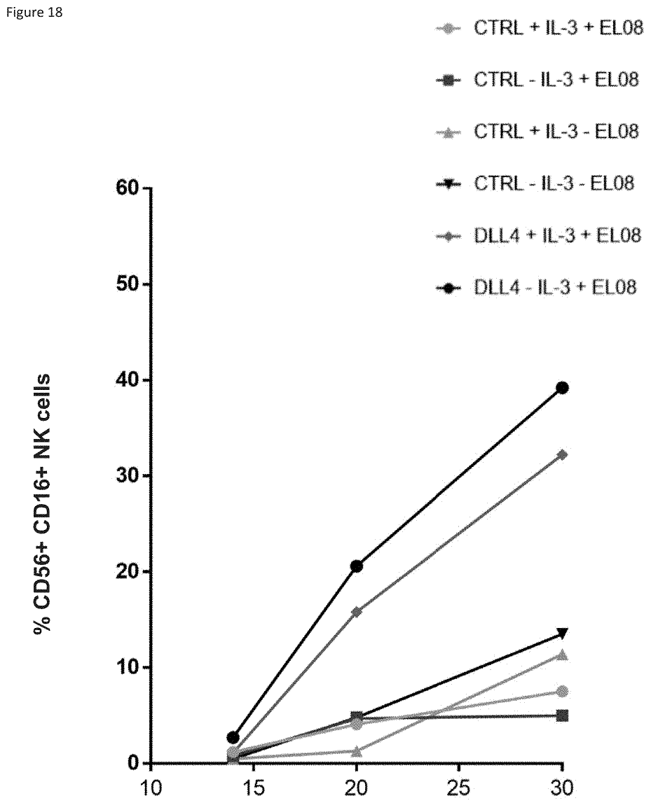

As shown in FIG. 18, treatment with DLL4 results in approximately 25% more CD56⁺CD16⁺ cells by day 30 compared to the respective control. Removal of IL-3 further increases the % of CD56⁺CD16⁺ cells in the absence of stromal cells or when cultured on DLL4 but not in the normal control (on stromal cells).

1. An ex vivo method for increasing the number of CD16⁺ NK cells in an expanded NK cell population, comprising the steps of:
(A) culturing an haematopoietic progenitor cell (HPC) comprising sample obtained from an individual in medium which does not induce differentiation of the HPCs, wherein the culturing is for between about 2 to about 8 days, to produce a pre-differentiation HPC population; and
(B) culturing the pre-differentiation HPC population in medium which induces differentiation of the HPCs to NK;
wherein the HPCs are cultured in the presence of a Notch ligand for at least part of step (A).

2. An ex vivo method for producing an expanded population of CD16⁺ Natural Killer (NK) cells, comprising the steps of:
(A) culturing an haematopoietic progenitor cell (HPC) comprising sample obtained from an individual in medium which does not induce differentiation of the HPCs, wherein the culturing is for between about 2 to about 8 days, to produce a pre-differentiation HPC population; and
(B) culturing the pre-differentiation HPC population in medium which induces differentiation of the HPCs to NK;
wherein the HPCs are cultured in the presence of a Notch ligand for at least part of step (A).

3. The method according to claim 1, wherein in step (A) the HPCs are cultured for between about 2 day to about 6 days, optionally for between about 4 days to about 6 days.

4. The method according to claim 1, wherein:
(i) the HPCs are cultured in a vessel that is coated with the Notch ligand for at least part of step (A); and/or
(ii) the Notch ligand is delta-like ligand 4 (DLL4), or a fragment thereof which retains the function of DLL4;
wherein preferably in step (A) the HPCs are cultured in the absence of the Notch ligand for about 1 day followed by culture in the presence of the Notch ligand for the remainder of step (A).

5. The method according to claim 1, wherein:
(i) in step (A) the HPCs are cultured in the presence of a compound which inhibits the action of REV-ERB; and/or
(ii) in step (B) the pre-differentiation HPC population is cultured in the presence of a compound which inhibits the action of REV-ERB.

6. The method according to claim 5, wherein said compound:
(i) increases E4bp4 expression by decreasing REV-ERB activity;
(ii) decreases the activity of REV-ERB-α and/or REV-ERB-β, preferably REV-ERB-β;
(iii) decreases the activity of REV-ERB-α and REV-ERB-β;
(iv) is a REV-ERB antagonist, preferably an antagonist of REV-ERB-α and REV-ERB-β;
(v) is selected from a small molecule, a PROTAC reagent, a double stranded RNA (dsRNA), a small interfering RNA (siRNA), a small hairpin RNA (shRNA), a micro RNA, an antisense RNA, an aptamer, an antibody, a ribozyme, a peptide or a peptidomimetic, preferably a small molecule; and/or
(vi) is SR8278, 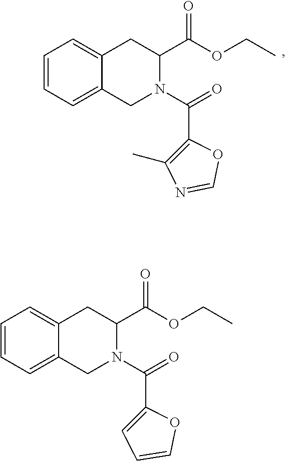 [SMILES: CCOC(=O)C1Cc2ccccc2CN1C(=O)c1ocnc1C ; CCOC(=O)C1Cc2ccccc2CN1C(=O)c1ccco1]
or GSK1362.

7. The method according to claim 1, wherein the medium which does not induces differentiation of the HPCs in step (A) and/or the medium which induces differentiation of the HPCs to NK in step (B) does not comprise IL-3, preferably wherein the medium which induces differentiation of the HPCs to NK in step (B) does not comprise IL-3.

8. The method according to claim 1, wherein:
(i) the medium in step (A) comprises at least one of Flt3L, GM-CSF, IL-3, IL-6, TPO and stem cell factor (SCF), preferably comprises each of Flt3L, GM-CSF, IL-3, IL-6, TPO and SCF; and/or
(ii) the medium in step (B) comprises at least one of IL-7, Flt3L, IL-15, and SCF, preferably comprises each of IL-7, Flt3L, IL-15 and SCF.

9. The method according to claim 1, wherein the step (A) and/or step (B) is carried out in the absence of a stromal support cell, preferably wherein both step (A) and step (B) are carried out in the absence of a stromal support cell.

10. The method according to claim 1, wherein the sample of HPCs is obtained from bone marrow, cord blood and/or peripheral blood.

11. The method according to claim 1, wherein the proportion of CD16⁺ NK cells is increased compared with the proportion of CD16⁺ NK cells produced by a corresponding method in which step (A) is omitted.

12. The method according to claim 1, wherein the expanded NK cell population comprises at least 10% CD16⁺ NK cells, preferably at least 15% CD16⁺ NK cells, more preferably at least 20% CD16⁺ NK cells, even more preferably at least 30% CD16⁺ NK cells.

13. The method according to claim 1, wherein the expanded NK cell population exhibits at least 30% greater antibody-dependent cellular cytotoxicity (ADCC), preferably at least 50% greater ADCC, compared with NK cells produced by a corresponding method in which step (A) is omitted.

14. The method according to claim 1, which does not comprise a step of introducing exogenous nucleic acid into the HPCs and/or NK cells.

15. An expanded population of CD16⁺NK cells, wherein at least 10% of the NK cells are CD16⁺ NK cells, preferably at least 15% of the NK cells are CD16⁺ NK cells, more preferably at least 20% of the NK cells are CD16⁺ NK cells, even more preferably at least 30% of the NK cells are CD16⁺ NK cells.

16. An expanded population of CD16⁺NK cells obtained by the method of claim 1, wherein at least 10% of the NK cells are CD16⁺ NK cells, preferably at least 15% of the NK cells are CD16⁺ NK cells, more preferably at least 20% of the NK cells are CD16⁺ NK cells, even more preferably at least 30% of the NK cells are CD16⁺ NK cells.

17. The expanded population of CD16⁺NK cells according to claim 15, wherein the CD16⁺ NK cells do not comprise exogenous nucleic acid.

18. The expanded population of CD16⁺NK cells according to claim 15, wherein the expanded NK cell population exhibits at least 30% greater ADCC, preferably at least 50% greater ADCC, more preferably at least 70% greater ADCC, compared with NK cells produced by a corresponding method in which step (A) is omitted.

19. A composition comprising an expanded NK cell population as defined in claim 15 and a pharmaceutically acceptable carrier, diluent and/or excipient.

20. A method of treatment comprising administering a therapeutically effective amount of an expanded population of CD16⁺NK cells as defined in claim 15 to a subject in need thereof.

21. The method of treatment of claim 20, wherein said method is a method of treating a disease or disorder selected from cancer, an infectious disease (acute or chronic), an autoimmune disease or a disease or disorder related to female infertility or pregnancy.

22. The method of treatment of claim 20, wherein said method is a method of treatment of a viral infection, a bacterial infection, a protist infection, a fungal infection and/or a helminth infection.

23. The method of treatment of claim 20, wherein the expanded population of CD16⁺ NK cells is used in combination with antibody-mediated immunotherapy.

24. The method of treatment of claim 23, wherein said expanded population of CD16⁺ NK cells is for administration before, simultaneously with, or after administration of the antibody-mediated immunotherapy.
